(19) United States
(12) Patent Application Publication
(10) Pub. No.: US 2026/0306544 A1
(43) Pub. Date: Oct. 8, 2026

(54) CD28H DOMAIN-CONTAINING CHIMERIC ANTIGEN RECEPTORS AND METHODS OF USE

(71) Applicant: The United States of America, as represented by the Secretary, Dept. of Health and Human Services, Bethesda, MD (US)

(72) Inventors: Eric O. Long, Rockville, MD (US); Xiaoxuan Zhuang, Rockville, MD (US)

(73) Assignee: The United States of America, as represented by the Secretary, Dept. of Health and Human Services, Bethesda, MD (US)

(21) Appl. No.: 19/707,856

(22) Filed: Jun. 15, 2026

Related U.S. Application Data
(62) Division of application No. 17/914,027, filed on Mar. 26, 2020, now Pat. No. 12,678,461.

(51) Int. Cl. A61K 35/17; A61K 38/00; A61P 35/00; C07K 14/705

(52) U.S. Cl. CPC A61K 35/17; A61P 35/00; C07K 14/70521; A61K 38/00

(57) ABSTRACT

Chimeric antigen receptors including (a) an antigen binding domain; (b) a transmembrane domain; and (c) an intracellular domain comprising a first intracellular signaling domain from CD28 homolog (CD28H) and a second intracellular signaling domain are provided. In some examples, the second intracellular domain is from 2B4, TCRζ, FcεR1γ, or DAP12. Chimeric antigen receptors including (a) an antigen binding domain; (b) a transmembrane domain; and (c) an intracellular domain comprising a first intracellular signaling domain from CD28H, a second intracellular signaling domain from 2B4, and a third intracellular signaling domain are also provided. In some examples, the third intracellular domain is from TCRζ, FcεR1γ, or DAP12. Nucleic acid molecules encoding the CARs and expression vectors including the nucleic acids are also provided. Isolated cells (such as T cells or natural killer cells) expressing the CARs and methods of treating a subject with cancer with the isolated cells are provided.

### CROSS REFERENCE TO RELATED APPLICATIONS

This application is a divisional of U.S. application Ser. No. 17/914,027, filed Sep. 23, 2022, which is the U.S. National Stage of International Application No. PCT/US2020/024985, filed Mar. 26, 2020, each of which are incorporated herein by reference in their entirety.

### ELECTRONIC SEQUENCE LISTING INCORPORATION

The Sequence Listing is submitted as an XML file in the form of the file named “4239-104206-10_Sequence_Listing” (50,108 bytes), which was created on Jun. 15, 2026 which is incorporated by reference herein.

### FIELD

This disclosure relates to chimeric antigen receptors containing a CD28H signaling domain and methods of their use, particularly for treating cancer.

### BACKGROUND

Immunotherapy with CAR-T cells has been successful in clinical treatment of hematologic cancers, and substantial progress has been made in targeting solid tumors. However, unique adverse side-effects associated with infusion of CAR-T cells, such as severe cytokine-release-syndrome (CRS) and neurotoxicity, could be life-threatening and need to be carefully managed. Natural killer cells have great potential as effector cells in adoptive cell therapy due to their safer cytokine profile and low graft-versus-host-disease (GVHD) activity, as observed in hematopoietic stem cell transplantation. To reduce the cost of CAR-T cell therapy and simplify the process of CAR-T cell production, “off-the-shelf” CAR-T cell strategies have been proposed. T cell receptors (TCRs) on allogeneic CAR-T cells derived from healthy donors have to be silenced in order to minimize GVHD and produce universal cryopreserved CAR-T cell products that are ready for use when needed. Notably, unlike T cells, “off-the-shelf” CAR-NK strategies can be developed without the need for genetically silencing TCR. Safety and persistence of donor-derived NK cells have been demonstrated by the decades-long clinical practice of hematopoietic stem cell transplantation. Moreover, additional beneficial effects can be provided by germline encoded NK-cell activation receptors for ligands on tumor cells.

Activation of NK cells requires a synergistic combination of signals from activation receptors, the ligands of which are usually upregulated on transformed or infected cells. However, activation signals are counteracted by inhibitory receptors for MHC-I, including receptor CD94-NKG2A for the non-classical MHC-I HLA-E and inhibitory killer-cell Ig-like receptors (KIRs) for classical MHC-I molecules. Specifically, KIR2DL1 is a receptor for HLA-C group 1 (C1), KIR2DL2/3 for HLA-C group 2 (C2), and KIR3DL1 for certain alleles of HLA-B. Signaling by these inhibitory receptors is typically dominant over signaling by co-activation receptors. Inhibitory receptors have also the important function of maintaining NK cells in a state of high-responsiveness, a property which has been called licensing or education.

### SUMMARY

In order for adoptive cell therapy with NK cells to succeed in elimination of tumor cells, signaling by inhibitory receptors needs to be reduced or overridden. Disclosed herein are CARs that include a CD28 homolog (CD28H) activation domain (also referred to as a CD28H intracellular signaling domain), which endows CARs with the ability to overcome inhibition, for example, by CD94-NKG2A and by KIR2DL1.

Disclosed are chimeric antigen receptors (CARs) including (a) an antigen binding domain; (b) a transmembrane domain; and (c) an intracellular domain, comprising a first intracellular signaling domain from CD28H and a second intracellular signaling domain. Domains (a)-(c) are in N-terminal to C-terminal order; however, the first and second intracellular signaling domains of the intracellular domain can be either order. In some examples, the second intracellular signaling domain is from 2B4, TCRζ, FcεR1γ, or DAP12. In one particular example, the second intracellular signaling domain is from TCRζ. In another particular example, the second intracellular signaling domain is from 2B4. In some embodiments, the CAR further includes a hinge domain (such as a CD8 hinge domain) that is C-terminal to the antigen binding domain and N-terminal to the transmembrane domain.

Also provided are chimeric antigen receptors (CARs) including (a) an antigen binding domain; (b) a transmembrane domain; and (c) an intracellular domain, comprising a first intracellular signaling domain from CD28H, a second intracellular signaling domain from 2B4, and a third intracellular signaling domain. In some examples, the third intracellular signaling domain is from TCRζ, FcεR1γ, or DAP12. Domains (a)-(c) are in N-terminal to C-terminal order; however, the first, second, and third intracellular signaling domains can be any order. In some embodiments, the CAR further includes a hinge domain (such as a CD8 hinge domain) that is C-terminal to the antigen binding domain and N-terminal to the transmembrane domain.

In some embodiments, the CARs disclosed herein include an amino acid sequence with at least 90% sequence identity to the amino acid sequence of any one of SEQ ID NOs: 2, 6, 12, 16, and 20. In particular examples, CARs include or consist of the amino acid sequence of any one of SEQ ID NOs: 2, 6, 12, 16, and 20.

Also disclosed are nucleic acid molecules encoding the CARs provided herein. In some embodiments, the nucleic acid molecule is included in an expression vector (such as a lentiviral or retroviral vector). In embodiments, the CARs include a nucleic acid sequence with at least 90% sequence identity to the nucleic acid sequence of any one of SEQ ID NOs: 1, 5, 11, 15, and 19 or include or consist of the nucleic acid sequence of any one of SEQ ID NOs: 1, 5, 11, 15, and 19. Isolated cells (such as T cells or natural killer cells) expressing the CARs are provided, as are compositions including the cells and a pharmaceutically acceptable carrier.

Further provided are methods of treating a subject with cancer (such as a hematological malignancy or solid tumor). Such methods include administering to the subject an isolated cell or composition disclosed herein. In some examples, the cells are NK cells expressing a CAR, such as autologous NK cells.

The foregoing and other features of the disclosure will become more apparent from the following detailed description, which proceeds with reference to the accompanying figures.

### BRIEF DESCRIPTION OF THE DRAWINGS

FIGS. 1A-1D show CD28H expression on human NK cells. FIG. 1A: Freshly isolated NK cells were stained for CD56 and CD28H with fluorophore-conjugated mAbs. An isotype control (IgG) for CD28H is shown in the left panel. FIG. 1B: Expression of CD28H was compared between CD56^{bright} and CD56^{dim} NK cells. Each symbol represents an independent donor (n=4). FIG. 1C: Expression of CD28H in different CD56^{dim} subsets defined by KIR and NKG2A expression. Receptors of the KIR family were stained using a cocktail of PE-conjugated antibodies (EB6, GL183 and DX9). Each symbol represents an independent donor (n=4) FIG. 1D: CD28H expression on CD56_{dim}CD57⁻ and CD56^{dim}CD57⁺ NK cells. Each symbol represents an independent donor (n=4). All data are presented as the mean±SEM. *P<0.05, n.s., not-significant (Mann-Whitney test, two-tailed). All experiments were repeated at least twice.

FIGS. 2A-2F. FIG. 2A: Gating strategy of different CD56^{dim} NK cell populations based on NKG2A and KIR expression. KIR expression was determined by a cocktail of PE-conjugated antibodies (EB6, GL183 and DX9). FIG. 2B: Representative histogram of immunostaining for CD28H in resting NK cells and NK cells expanded in IL-2 and PHA for 14 days. Freshly isolated or IL-2 cultured NK cells were stained with isotype control (shaded) or CD28H mAb (solid line), followed by incubation with PE-conjugated goat anti-mouse secondary antibody. FIG. 2C: Representative histogram of immunostaining for CD28H in resting NK cells (left) or NK cells activated by IL-2 and crosslinking of CD2 and NKp46 (right). NK cells were activated by plate-bound mAbs to CD2 and NKp46 plus 100 U/ml IL-2 for 7 days. FIG. 2D: Expression of CD28H at different time points after activation of NK cells from 2 donors as in FIG. 2C. The percentage of NK cells that were CD28H⁺ was determined by staining for CD56 and CD28H. FIG. 2E: CD25 expression on NK cells at different time points after IL-2 stimulation. FIG. 2F: Resting NK cells were activated in IL-2 for 24 hours, followed by staining for CD56, CD25 and CD28H. Expression of CD28H in CD25⁻ and CD25⁺ NK cells shown in histogram (right) and presented as data from 3 independent donors (left). Mean±SEM. n.s., not-significant (Mann-Whitney test, two-tailed).

FIGS. 3A-3C. FIG. 3A: Schematics of redirected cytotoxicity assay. In the assay, the F(ab′)₂ portions of the mAbs specifically recognize NK cell receptors, while the Fc fragments bind to Fc receptor on mouse P815 cells. NK cells are synergistically activated by the crosslinked NK cell activation receptors. FIG. 3B: Surface expression of 2B4, NKp46, and CD28H in the NKL, YTS, KHYG-1, and NK-92 cell lines determined by immunostaining with fluorophore-conjugated mAbs. Shaded histograms represent staining with isotype controls. FIG. 3C: Lysis of P815 cells by KHYG-1 cells in redirected cytotoxicity assays. KHYG-1 cells were rested without IL-2 for 24 hours before incubation with P815 cells and the indicated mAbs for 6 hours at E to T ratios of 1 and 5.

FIGS. 4A-4K show that CD28H synergizes with 2B4 and NKp46 for NK-cell activation and enhances activation through CD16. FIG. 4A: Representative contour plots of NK-cell degranulation induced by CD28H, 2B4, and NKp46, either alone or in combination, in redirected cytotoxicity assays. Freshly isolated NK cells were incubated with P815 cells and 5 μg/ml the indicated mAbs at 37° C. for 2 hours. The E to T ratio was 1:2. NK-cell degranulation was determined by staining for CD107a. A mAb to CD56 was a negative control for degranulation, whereas NKp46 and 2B4 co-engagement was a positive control for synergy of NK activating receptors. FIG. 4B: NK-degranulation as in FIG. 4A from several donors. Each symbol represents an independent donor (n=5). FIGS. 4C-4F: NK-cell degranulation upon co-engagement of CD28H with CD2 (FIG. 4C), DNAM-1 (FIG. 4D), NKG2D (FIG. 4E), and CD16 (FIG. 4F) in redirected cytotoxicity assays performed as in FIG. 4A. Each symbol represents an independent donor, n=4 in FIG. 4C, n=3 in FIG. 4D, n=4 in FIG. 4E, and n=5 in FIG. 4F. FIGS. 4G-4I: NK-cell degranulation as in FIG. 4A in redirected cytotoxicity assays. A fixed concentration (5 μg/ml) of mAbs to 2B4 (FIG. 4G), NKp46 (FIG. 4H) and CD16 (FIG. 4I) was used, and CD28H antibody was added at increasing concentrations. Each symbol represents an independent donor, n=5 in FIG. 4G, n=5 in FIG. 4H, n=6 in FIG. 4I. FIGS. 4J-4K: NK cells incubated with P815 cells and the indicated mAbs for 6 hours at various E to T ratios. Lysis of P815 cells induced by co-engagement of CD28H with 2B4 (FIG. 4J) or NKp46 (FIG. 4K); each graph represents one of two independent experiments. All data are presented as the mean±SEM. *P<0.05, ** P<0.01, n.s., not-significant (Mann-Whitney test, two-tailed). All experiments were repeated at least twice.

FIGS. 5A and 5B show NK cell degranulation and cytokine production induced by B7H7 and CD48 on Drosophila S2 cells. FIG. 5A: Expression of CD48 and B7H7 on S2 cells transfected with CD48, B7H7, or both. Shaded histogram indicates staining of untransfected S2 cells. FIG. 5B: Pie charts represent the frequency of NK cells positive for the indicated number of responses. Arcs represent the proportion of NK cells positive for MIP-1α, IFN-γ, TNF-α and CD107a. Values represent mean of 3 donors.

FIGS. 6A-6F show NK-cell degranulation and cytokine production induced by B7H7 and CD48 on Drosophila S2 cells. FIG. 6A: Representative contour plots of NK-cell degranulation induced by S2 cells and S2 cells expressing B7H7, CD48, or both, after incubation for 2 hours at an E:T ratio of 1:2. Degranulation was determined by staining for CD56 and CD107a. FIG. 6B: NK-cell degranulation as in FIG. 6A from several donors. Each symbol represents an independent donor, n=5. FIGS. 6C-6F: Cytokine production by NK cells after incubation with the indicated S2 cells at 37° C. for 6 hours at an E:T ratio of 1:2. A mAb to CD28H (anti-CD28H) was included at 10 μg/ml to block the B7H7-CD28H interaction. Cells were stained for CD56 and CD107a, fixed and permeabilized, and stained for intracellular cytokines. Expression of IFNγ (FIG. 6C), TNFα (FIG. 6D), MIP-1α (FIG. 6E) and MIP-1β (FIG. 6F) in NK cells from multiple donors. Each symbol represents an independent donor, n=7 in FIG. 6C, n=5 in FIG. 6D, n=6 in FIG. 6E, and n=6 in FIG. 6F. All data are presented as the mean±SEM. *P<0.05, **P<0.01, ***P<0.001 (Mann-Whitney test, two-tailed). All experiments have been repeated at least twice.

FIGS. 7A-7E show that B7H7 on target cells enhances ADCC by NK cells. FIG. 7A: Representative contour plots of NK-cell degranulation induced by the indicated S2 cells in the presence of anti-S2 serum for 2 hours and measured by staining with fluorophore-conjugated CD56 and CD107a mAbs. FIG. 7B: Degranulation by NK cells as in FIG. 7A from multiple donors. Each symbol represents an independent donor, n=7. FIG. 7C: Staining for CD20 on 221 and Daudi cells. Shaded histograms indicate staining with isotype control. FIGS. 7D and 7E: Specific lysis of target cells by NK cells at effector to target cell ratio of 5:1 in the presence of 10 μg/ml Rituximab. Each symbol represents an independent donor, n=5 in both FIG. 7D and FIG. 7E. Target cells were 221 (FIG. 7D) and Daudi (FIG. 7E) cell lines, either untransfected or transfected with B7H7. A mAb to CD28H (anti-CD28H) was included at 10 μg/ml to block the B7H7-CD28H interaction. All data are presented as the mean±SEM. *P<0.05 (Wilcoxon signed-rank test, paired, two-tailed). All experiments were repeated at least twice.

FIGS. 8A-8C show that B7H7 on 221 cells promotes CD28H-dependent lysis by NK cells. FIG. 8A: Staining for B7H7 and CD48 on Daudi, K562, and 221 cells. Shaded histograms represent staining with isotype controls. FIG. 8B: Expression of B7H7 on transfected 221 and Daudi cells. Shaded histograms represent staining of untransfected cells. FIG. 8C: Lysis of 221 and 221.B7H7 cells by resting NK cells at the indicated E to T ratios after 6 hours. A mAb to CD28H was added at 10 μg/ml to block the CD28H-B7H7 interaction (square symbols).

FIGS. 9A-9F illustrate that Tyr192 of CD28H is essential for CD28H-mediated NK-cell activation.

FIG. 9A: Tyrosine-phosphorylation of CD28H wild type and the indicated Tyr mutants upon pervanadate treatment. Transfected 293T cells were treated with pervanadate for 10 minutes. Cell lysates were immunoprecipitated with CD28H mAb. Phospho-tyrosine (4G10) and total CD28H (anti-2A) were detected by immunoblots. FIG. 9B: Lysis of P815 cells by NKL.CD28H cells at various E:T ratios in presence of the indicated mAbs after 6 hours. FIG. 9C: Lysis of P815 cells by NKL cell transfectants in presence of mAbs to 2B4 and CD28H at the indicated E:T ratios. NKL.CD28H-WT with mAb to 2B4 served as negative control (Ctrl). The graph represents one of two independent experiments. FIG. 9D: Lysis of 221.B7H7 cells by NKL cell transfectants at the indicated E:T ratios after 6 hours. Untransfected 221 cells served as negative control. The graph represents one of two independent experiments. FIG. 9E: Immunofluorescence staining with CD28H antibody of Daudi or Daudi-B7H7 cells incubated with NKL cells expressing CD28H wild-type or Y192F mutant. FIG. 9F: Fold change in fluorescence intensity of CD28H at the immunological synapse (IS), as compared to cell surface regions away from the synapse (non-IS), as in FIG. 9E. Each dot represents a single cell. Sample size (n) is shown in the figure. Data were obtained and combined from two independent experiments and presented as the mean±SEM. ****P<0.0001 (Mann-Whitney test, two-tailed). All experiments were repeated at least twice.

FIG. 10 shows CD28H expression on NKL cells transfected with the indicated CD28H mutants. Shaded histograms represent staining of untransfected cells.

FIGS. 11A and 11B show B7H7 expression on circulating monocytes and myeloid dendritic cells. FIG. 11A: Either untreated or LPS- and poly(I:C)-stimulated human PBMC were gated on the CD14⁺ population and tested for B7H7 expression with fluorophore-conjugated B7H7 mAb. FIG. 11B: Expression of B7H7 in circulating myeloid dendritic cells. Human PBMC treated as in FIG. 11A were gated on lineage-negative (CD3-CD19⁻CD14⁻NKp46⁻) CD11c⁺ cells, and tested for expression of B7H7 by staining with B7H7 mAb. Shaded histograms represent staining with isotype control.

FIGS. 12A-12J show lysis of B7H7⁺ HLA-E⁺ HDLM-2 tumor cells by NKG2A⁺ NK cells expressing a CD28H-TCRζ chimeric antigen receptor. FIG. 12A: Design and expression of CD28H chimeric antigen receptors in NKL cells. Full-length or cytoplasmic-domain-deleted (ΔCD) CD28H was fused to the cytoplasmic domain of TCRζ and transfected into NKL cells. Cells were stained with fluorophore-conjugated CD28H mAb. Shaded histograms represent staining of untransfected NKL cells. FIGS. 12B-12C: Lysis of 221 cells (FIG. 12B) and 221.B7H7 cells (FIG. 12C) by transfected NKL cells at the indicated E:T ratio after 5 hours. FIG. 12D: Staining for HLA-E and B7H7 in 221.AEH and 221.AEH.B7H7 cells. Shaded histograms represent staining of untransfected 221 cells. FIG. 12E: Expression of NKG2A on NKL cells determined by immunostaining. Shaded histograms represent staining with IgG control. FIGS. 12F-12G: Lysis of 221.AEH cells (FIG. 12F) and 221.AEH.B7H7 cells (FIG. 12G) by transfected NKL cells at the indicated E:T ratios after 5 hours. The graph represents one of two independent experiments. FIG. 12H: Staining of B7H7 and HLA-E on HDLM-2 tumor cells. Shaded histograms represent staining with isotype controls. FIG. 12I: Expression of NKG2A on KHYG-1 cells determined by immunostaining (left). Lysis of HDLM-2 tumor cells by KHYG-1 cells after 5 hours, in the presence or absence of a blocking antibody to NKG2A (right). The graph represents one of two independent experiments. FIG. 12J: Lysis of HHLA-2⁺ HLA-E⁺ HDLM-2 tumor cells by transfected NKL cells at the indicated E:T ratio after 5 hours. All experiments have been repeated at least twice.

FIGS. 13A-13E show synergistic activation of NK cell degranulation by combinations of activating receptors, the design and expression of CD19.CARs, and lysis of CD19⁺ MHC-I⁺ tumor cells by NK cells expressing designed CD19.CARs. FIG. 13A: Degranulation of resting human NK cells induced by crosslinking combinations of NK-cell activating receptors. Crosslinking was performed in a redirected cytotoxicity assay using P815 cells in the presence of antibodies for the indicated activating receptors (n=2). FIG. 13B: Schematic diagram of CAR constructs using anti-CD19 scFv with a N-terminal myc-tag and a CD8 ┐ hinge as extracellular domain and the indicated combinations of transmembrane and signaling domains. FIG. 13C: Histograms showing expression of CAR constructs in NKL.2DL1 cells. FIG. 13D: Killing assays comparing lysis of target cells by the indicated CAR-expressing NKL.2DL1 cells. Either the mutated MHC-I negative lymphoblastoid cell line 221, or MHC-I (HLA-E or HLA-C/Cw15) transfected 221 cells were used as target (n=3). FIG. 13E: Comparing target-cell lysis by NKL.2DL1 cells expressing CAR2 or T-CAR (n=3). Same target cells were used as in FIG. 13D.

FIG. 14 is a pair of panels showing expression of KIR2DL1 (left) and NKG2A (right) on NKL.2DL1 cells. NKL.2DL1 cells were stained with IgG controls (shaded), and IgG antibodies to KIR2DL1 (left, open histogram) and NKG2A (right, open histogram).

FIGS. 15A-15D show lysis of B7H7⁺ MHC-I⁺ tumor cells by NK cells expressing a CD28H-CAR, compared to NK cells expressing a 3^{rd} generation T-CAR or an NKG2D-2B4-TCRζ CAR. FIG. 15A: Schematic diagram of CAR constructs consisting of the extracellular domain of CD28H and the indicated combinations of signaling domains. FIG. 15B: Flow cytometry determination of expression of CD28H.T-CAR, CD28H.CAR2 and CD28H.CAR3 in NKL.2DL1 cells. FIG. 15C: Killing assays using CAR-transduced NKL.2DL1 cells as effector and 221.HLA-E cells or B7H7-transfected 221.HLA-E cells as targets (n=2). FIG. 15D: Lysis of 221.HLA-C(Cw15) cells or B7H7-transfected 221.HLA-C(Cw15) cells by NKL.2DL1 cells expressing CD28H.CARs (n=2).

FIGS. 16A-16E illustrates that CD19.CAR2 overcomes NKG2A-mediated inhibition and transduces more persistent activation signals than T-CAR. FIG. 16A: Diagram of CAR stimulation by antibodies immobilized on beads. FIG. 16B: Western blot detection of activation signals in lysates of CARs-expressing NKL cells after stimulation by beads. The indicated antibodies were pre-bound to beads, and then incubated with CAR-NK cells for 10 min. Cell lysates were probed for the indicated signaling molecules. FIG. 16C: Western blot quantification of pErk/total-Erk and pPLCg1/total-PLCg1 ratios (n=2). FIG. 16D: Bead stimulation of NKL cells expressing T-CAR or CAR2. Co-incubation of beads and NK cells were performed for 10 minutes and 45 minutes, as indicated. Cell lysates were Western blotted with the indicated antibodies. FIG. 16E: Calcium mobilization induced in NKL cells expressing T-CAR or CAR2 after CAR-crosslinking with antibodies. NKL cells were preincubated with Myc-tag antibody on ice for 30 min and incubated at 37° C. for 5 min before the assay. Baseline calcium was measured for 30 s before addition of secondary goat-anti-mouse IgG antibodies for crosslinking.

FIG. 17 shows stimulation of NKL cells expressing T-CAR and the CAR2 to CAR6 shown in FIG. 13B (N: no CAR). Beads coupled with the indicated antibodies were incubated with CAR-NK cells for 10 min at 37° C. Cell lysates were Western-blotted with antibodies for phosphorylated or total Erk and PLC-1, as indicated. Quantification of band intensity is shown in FIG. 16C.

### SEQUENCE LISTING

Any nucleic acid and amino acid sequences listed herein or in the accompanying sequence listing are shown using standard letter abbreviations for nucleotide bases and amino acids, as defined in 37 C.F.R. § 1.822. In at least some cases, only one strand of each nucleic acid sequence is shown, but the complementary strand is understood as included by any reference to the displayed strand.

SEQ ID NO: 1 is the nucleic acid sequence of full-length CD28H fused with the cytoplasmic domain of TCRζ. CD28H: nucleotides 1-846; TCRζ: nucleotides 847-1185.

ATGGGGTCCCCGGGCATGGTGCTGGGCCTCCTGGTGCAGATCTGGGCCCT
GCAAGAAGCCTCAAGCCTGAGCGTGCAGCAGGGGCCCAACTTGCTGCAGG
TGAGGCAGGGCAGTCAGGCGACCCTGGTCTGCCAGGTGGACCAGGCCACA
GCCTGGGAACGGCTCCGTGTTAAGTGGACAAAGGATGGGGCCATCCTGTG
TCAACCGTACATCACCAACGGCAGCCTCAGCCTGGGGGTCTGCGGGCCCC
AGGGACGGCTCTCCTGGCAGGCACCCAGCCATCTCACCCTGCAGCTGGAC
CCTGTGAGCCTCAACCACAGCGGGGCGTACGTGTGCTGGGCGGCCGTAGA
GATTCCTGAGTTGGAGGAGGCTGAGGGCAACATAACAAGGCTCTTTGTGG
ACCCAGATGACCCCACACAGAACAGAAACCGGATCGCAAGCTTCCCAGGA
TTCCTCTTCGTGCTGCTGGGGGTGGGAAGCATGGGTGTGGCTGCGATCGT
GTGGGGTGCCTGGTTCTGGGGCCGCCGCAGCTGCCAGCAAAGGGACTCAG
GTAACAGCCCAGGAAATGCATTCTACAGCAACGTCCTATACCGGCCCCGG
GGGGCCCCAAAGAAGAGTGAGGACTGCTCTGGAGAGGGGAAGGACCAGAG
GGGCCAGAGCATTTATTCAACCTCCTTCCCGCAACCGGCCCCCCGCCAGC
CGCACCTGGCGTCAAGACCCTGCCCCAGCCCGAGACCCTGCCCCAGCCCC
AGGCCCGGCCACCCCGTCTCTATGGTCAGGGTCTCTCCTAGACCAAGCCC
CACCCAGCAGCCGAGGCCAAAAGGGTTCCCCAAAGTGGGAGAGGAGagag
tgaagttcagcaggagcgcagacgcccccgcgtaccagcagggccagaac
cagctctataacgagctcaatctaggacgaagagaggagtacgatgtttt
ggacaagagacgtggccgggaccctgagatggggggaaagccgcagagaa
ggaagaaccctcaggaaggcctgtacaatgaactgcagaaagataagatg
gcggaggcctacagtgagattgggatgaaaggcgagcgccggaggggcaa
ggggcacgatggcctttaccagggtctcagtacagccaccaaggacacct
acgacgcccttcacatgcaggccctgccccctcgc

SEQ ID NO: 2 is the amino acid sequence of full-length CD28H fused with the cytoplasmic domain of TCRζ. CD28H: amino acids 1-282; TCRζ: amino acids 283-395.

MGSPGMVLGLLVQIWALQEASSLSVQQGPNLLQVRQGSQATLVCQVDQAT
AWERLRVKWTKDGAILCQPYITNGSLSLGVCGPQGRLSWQAPSHLTLQLD
PVSLNHSGAYVCWAAVEIPELEEAEGNITRLFVDPDDPTQNRNRIASFPG
FLFVLLGVGSMGVAAIVWGAWFWGRRSCQQRDSGNSPGNAFYSNVLYRPR
GAPKKSEDCSGEGKDQRGQSIYSTSFPQPAPRQPHLASRPCPSPRPCPSP
RPGHPVSMVRVSPRPSPTQQPRPKGFPKVGEERVKFSRSADAPAYQQGQN
QLYNELNLGRREEYDVLDKRRGRDPEMGGKPQRRKNPQEGLYNELQKDKM
AEAYSEIGMKGERRRGKGHDGLYQGLSTATKDTYDALHMQALPPR

SEQ ID NO: 3 is the nucleic acid sequence of extracellular and transmembrane domains of CD28H fused with the cytoplasmic domain of TCRζ. CD28H: nucleotides 1-513; TCRζ: nucleotides 514-852.

ATGGGGTCCCCGGGCATGGTGCTGGGCCTCCTGGTGCAGATCTGGGCCCT
GCAAGAAGCCTCAAGCCTGAGCGTGCAGCAGGGGCCCAACTTGCTGCAGG
TGAGGCAGGGCAGTCAGGCGACCCTGGTCTGCCAGGTGGACCAGGCCACA
GCCTGGGAACGGCTCCGTGTTAAGTGGACAAAGGATGGGGCCATCCTGTG
TCAACCGTACATCACCAACGGCAGCCTCAGCCTGGGGGTCTGCGGGCCCC
AGGGACGGCTCTCCTGGCAGGCACCCAGCCATCTCACCCTGCAGCTGGAC
CCTGTGAGCCTCAACCACAGCGGGGCGTACGTGTGCTGGGCGGCCGTAGA
GATTCCTGAGTTGGAGGAGGCTGAGGGCAACATAACAAGGCTCTTTGTGG
ACCCAGATGACCCCACACAGAACAGAAACCGGATCGCAAGCTTCCCAGGA
TTCCTCTTCGTGCTGCTGGGGGTGGGAAGCATGGGTGTGGCTGCGATCGT
GTGGGGTGCCTGGagagtgaagttcagcaggagcgcagacgcccccgcgt
accagcagggccagaaccagctctataacgagctcaatctaggacgaaga
gaggagtacgatgttttggacaagagacgtggccgggaccctgagatggg
gggaaagccgcagagaaggaagaaccctcaggaaggcctgtacaatgaac
tgcagaaagataagatggcggaggcctacagtgagattgggatgaaaggc
gagcgccggaggggcaaggggcacgatggcctttaccagggtctcagtac
agccaccaaggacacctacgacgcccttcacatgcaggccctgccccctc
gc

SEQ ID NO: 4 is the amino acid sequence of extracellular and transmembrane domains of CD28H fused with the cytoplasmic domain of TCRζ. CD28H: amino acids 1-171; TCRζ: amino acids 172-284.

MGSPGMVLGLLVQIWALQEASSLSVQQGPNLLQVRQGSQATLVCQVDQAT
AWERLRVKWTKDGAILCQPYINGSLSLGVCGPQGRLSWQAPSHLTLQLDP
VSLNHSGAYVCWAAVEIPELEEAEGNITRLFVDPDDPTQNRNRIASFPGF
LFVLLGVGSMGVAAIVWGAWRVKFSRSADAPAYQQGQNQLYNELNLGRRE
EYDVLDKRRGRDPEMGGKPQRRKNPQEGLYNELQKDKMAEAYSEIGMKGE
RRRGKGHDGLYQGLSTATKDTYDALHMQALPPR

SEQ ID NO: 5 is the nucleic acid sequence of full-length CD28H fused with the intracellular domains of 2B4 and TCRζ. The DNA sequence of CD28H was codon-optimized for synthesis as a gBlock fragment. CD28H: nucleotides 1-846; 2B4: nucleotides 847-1206; TCRζ: nucleotides 1207-1545.

ATGGGGTCCCCGGGCATGGTGCTGGGCCTCCTGGTGCAGATCTGGGCCCT
GCAAGAAGCCTCAAGCCTGAGCGTGCAGCAGGGGCCCAACTTGCTGCAGG
TGAGGCAGGGCAGTCAGGCGACCCTGGTCTGCCAGGTGGACCAGGCCACA
GCCTGGGAACGGCTCCGTGTTAAGTGGACAAAGGATGGGGCCATCCTGTG
TCAACCGTACATCACCAACGGCAGCCTCAGCCTGGGGGTCTGCGGGCCCC
AGGGACGGCTCTCCTGGCAGGCACCCAGCCATCTCACCCTGCAGCTGGAC
CCTGTGAGCCTCAACCACAGCGGGGCGTACGTGTGCTGGGCGGCCGTAGA
GATTCCTGAGTTGGAGGAGGCTGAGGGCAACATAACAAGGCTCTTTGTGG
ACCCAGATGACCCCACACAGAACAGAAACCGGATCGCAAGCTTCCCAGGA
TTCCTCTTCGTGCTGCTGGGGGTGGGAAGCATGGGTGTGGCTGCGATCGT
GTGGGGTGCCTGGTTCTGGGGCCGCCGCAGCTGCCAGCAAAGGGACTCAG
GTAACAGCCCAGGAAATGCATTCTACAGCAACGTCCTATACCGGCCCCGG
GGGGCCCCAAAGAAGAGTGAGGACTGCTCTGGAGAGGGGAAGGACCAGAG
GGGCCAGAGCATTTATTCAACCAGTTTCCCTCAACCGGCTCCAAGACAAC
CACATCTCGCCAGTCGGCCTTGTCCGTCCCCTAGACCCTGCCCCAGTCCC
AGGCCCGGCCACCCCGTCTCTATGGTCAGGGTCTCTCCTAGACCAAGCCC
CACCCAGCAGCCGAGGCCAAAAGGGTTCCCCAAAGTGGGAGAGGAGTGGA
GGAGAAAGAGGAAGGAGAAGCAGTCAGAGACCAGTCCCAAGGAATTTTTG
ACAATTTACGAAGATGTCAAGGATCTGAAAACCAGGAGAAATCACGAGCA
GGAGCAGACTTTTCCTGGAGGGGGGAGCACCATCTACTCTATGATCCAGT
CCCAGTCTTCTGCTCCCACGTCACAAGAACCTGCATATACATTATATTCA
TTAATTCAGCCTTCCAGGAAGTCTGGATCCAGGAAGAGGAACCACAGCCC
TTCCTTCAATAGCACTATCTATGAAGTGATTGGAAAGAGTCAACCTAAAG
CCCAGAACCCTGCTCGATTGAGCCGCAAAGAGCTGGAGAACTTTGATGTT
TATTCCagagtgaagttcagcaggagcgcagacgcccccgcgtaccagca
gggccagaaccagctctataacgagctcaatctaggacgaagagaggagt
acgatgttttggacaagagacgtggccgggaccctgagatggggggaaag
ccgcagagaaggaagaaccctcaggaaggcctgtacaatgaactgcagaa
agataagatggcggaggcctacagtgagattgggatgaaaggcgagcgcc
ggaggggcaaggggcacgatggcctttaccagggtctcagtacagccacc
aaggacacctacgacgcccttcacatgcaggccctgccccctcgc

SEQ ID NO: 6 is the amino acid sequence of full-length CD28H fused with the intracellular domains of 2B4 and TCRζ. CD28H: amino acids 1-282; 2B4: amino acids 283-402; TCKζ: amino acids 403-515.

MGSPGMVLGLLVQIWALQEASSLSVQQGPNLLQVRQGSQATLVCQVDQAT
AWERLRVKWTKDGAILCQPYITNGSLSLGVCGPQGRLSWQAPSHLTLQLD
PVSLNHSGAYVCWAAVEIPELEEAEGNITRLFVDPDDPTQNRNRIASFPG
FLFVLLGVGSMGVAAIVWGAWFWGRRSCQQRDSGNSPGNAFYSNVLYRPR
GAPKKSEDCSGEGKDQRGQSIYSTSFPQPAPRQPHLASRPCPSPRPCPSP
RPGHPVSMVRVSPRPSPTQQPRPKGFPKVGEEWRRKRKEKQSETSPKEFL
TIYEDVKDLKTRRNHEQEQTFPGGGSTIYSMIQSQSSAPTSQEPAYTLYS
LIQPSRKSGSRKRNHSPSFNSTIYEVIGKSQPKAQNPARLSRKELENFDV
YSRVKFSRSADAPAYQQGQNQLYNELNLGRREEYDVLDKRRGRDPEMGGK
PQRRKNPQEGLYNELQKDKMAEAYSEIGMKGERRRGKGHDGLYQGLSTAT
KDTYDALHMQALPPR

SEQ ID NO: 7 is the nucleic acid sequence of extracellular domain of CD28H fused with the transmembrane domain of CD28 and intracellular domains of CD28, 41BB, and TCRζ. CD28H: nucleotides 1-450; CD28: nucleotides 451-654; 4-1BB: nucleotides 655-780; TCRζ: nucleotides 781-1119.

ATGGGGTCCCCGGGCATGGTGCTGGGCCTCCTGGTGCAGATCTGGGCCCT
GCAAGAAGCCTCAAGCCTGAGCGTGCAGCAGGGGCCCAACTTGCTGCAGG
TGAGGCAGGGCAGTCAGGCGACCCTGGTCTGCCAGGTGGACCAGGCCACA
GCCTGGGAACGGCTCCGTGTTAAGTGGACAAAGGATGGGGCCATCCTGTG
TCAACCGTACATCACCAACGGCAGCCTCAGCCTGGGGGTCTGCGGGCCCC
AGGGACGGCTCTCCTGGCAGGCACCCAGCCATCTCACCCTGCAGCTGGAC
CCTGTGAGCCTCAACCACAGCGGGGCGTACGTGTGCTGGGCGGCCGTAGA
GATTCCTGAGTTGGAGGAGGCTGAGGGCAACATAACAAGGCTCTTTGTGG
ACCCAGATGACCCCACACAGAACAGAAACCGGATCGCAAGCTTCCCAGGA
TTTTGGGTGCTGGTGGTGGTTGGTGGAGTCCTGGCTTGCTATAGCTTGCT
AGTAACAGTGGCCTTTATTATTTTCTGGGTGAGGAGTAAGAGGAGCAGGC
TCCTGCACAGTGACTACATGAACATGACTCCCCGCCGCCCCGGGCCCACC
CGCAAGCATTACCAGCCCTATGCCCCACCACGCGACTTCGCAGCCTATCG
CTCCAAACGGGGCAGAAAGAAACTCCTGTATATATTCAAACAACCATTTA
TGAGACCAGTACAAACTACTCAAGAGGAAGATGGCTGTAGCTGCCGATTT
CCAGAAGAAGAAGAAGGAGGATGTGAACTGagagtgaagttcagcaggag
cgcagacgcccccgcgtaccagcagggccagaaccagctctataacgagc
tcaatctaggacgaagagaggagtacgatgttttggacaagagacgtggc
cgggaccctgagatggggggaaagccgcagagaaggaagaaccctcagga
aggcctgtacaatgaactgcagaaagataagatggcggaggcctacagtg
agattgggatgaaaggcgagcgccggaggggcaaggggcacgatggcctt
taccagggtctcagtacagccaccaaggacacctacgacgcccttcacat
gcaggccctgccccctcgc

SEQ ID NO: 8 is the amino acid sequence of extracellular domain of CD28H fused with the transmembrane domain of CD28 and intracellular domains of CD28, 4-1BB, and TCRζ. CD28H: amino acids 1-150; CD28: amino acids 151-218; 4-1BB: amino acids 219-260; TCRζ: nucleotides 261-373.

MGSPGMVLGLLVQIWALQEASSLSVQQGPNLLQVRQGSQATLVCQVDQAT
AWERLRVKWTKDGAILCQPYITNGSLSLGVCGPQGRLSWQAPSHLTLQLD
PVSLNHSGAYVCWAAVEIPELEEAEGNITRLFVDPDDPTQNRNRIASFPG
FWVLVVVGGVLACYSLLVTVAFIIFWVRSKRSRLLHSDYMNMTPRRPGPT
RKHYQPYAPPRDFAAYRSKRGRKKLLYIFKQPFMRPVQTTQEEDGCSCRF
PEEEEGGCELRVKFSRSADAPAYQQGQNQLYNELNLGRREEYDVLDKRRG
RDPEMGGKPQRRKNPQEGLYNELQKDKMAEAYSEIGMKGERRRGKGHDGL
YQGLSTATKDTYDALHMQALPPR

SEQ ID NO: 9 is the nucleic acid sequence of extracellular domain of CD28H fused with the transmembrane domain of NKG2D and intracellular domains of 2B4 and TCRζ. CD28H: nucleotides 1-450; NKG2D: nucleotides 451-513; 2B4: nucleotides 514-873; TCRζ: nucleotides 874-1212.

ATGGGGTCCCCGGGCATGGTGCTGGGCCTCCTGGTGCAGATCTGGGCCCT
GCAAGAAGCCTCAAGCCTGAGCGTGCAGCAGGGGCCCAACTTGCTGCAGG
TGAGGCAGGGCAGTCAGGCGACCCTGGTCTGCCAGGTGGACCAGGCCACA
GCCTGGGAACGGCTCCGTGTTAAGTGGACAAAGGATGGGGCCATCCTGTG
TCAACCGTACATCACCAACGGCAGCCTCAGCCTGGGGGTCTGCGGGCCCC
AGGGACGGCTCTCCTGGCAGGCACCCAGCCATCTCACCCTGCAGCTGGAC
CCTGTGAGCCTCAACCACAGCGGGGCGTACGTGTGCTGGGCGGCCGTAGA
GATTCCTGAGTTGGAGGAGGCTGAGGGCAACATAACAAGGCTCTTTGTGG
ACCCAGATGACCCCACACAGAACAGAAACCGGATCGCAAGCTTCCCAGGA
CCATTTTTTTTCTGCTGCTTCATCGCTGTAGCCATGGGAATCCGTTTCAT
TATTATGGTAACATGGAGGAGAAAGAGGAAGGAGAAGCAGTCAGAGACCA
GTCCCAAGGAATTTTTGACAATTTACGAAGATGTCAAGGATCTGAAAACC
AGGAGAAATCACGAGCAGGAGCAGACTTTTCCTGGAGGGGGGAGCACCAT
CTACTCTATGATCCAGTCCCAGTCTTCTGCTCCCACGTCACAAGAACCTG
CATATACATTATATTCATTAATTCAGCCTTCCAGGAAGTCTGGATCCAGG
AAGAGGAACCACAGCCCTTCCTTCAATAGCACTATCTATGAAGTGATTGG
AAAGAGTCAACCTAAAGCCCAGAACCCTGCTCGATTGAGCCGCAAAGAGC
TGGAGAACTTTGATGTTTATTCCagagtgaagttcagcaggagcgcagac
gcccccgcgtaccagcagggccagaaccagctctataacgagctcaatct
aggacgaagagaggagtacgatgttttggacaagagacgtggccgggacc
ctgagatggggggaaagccgcagagaaggaagaaccctcaggaaggcctg
tacaatgaactgcagaaagataagatggcggaggcctacagtgagattgg
gatgaaaggcgagcgccggaggggcaaggggcacgatggcctttaccagg
gtctcagtacagccaccaaggacacctacgacgcccttcacatgcaggcc
ctgccccctcgc

SEQ ID NO: 10 is the amino acid sequence of extracellular domain of CD28H fused with the transmembrane domain of NKG2D and intracellular domains of 2B4 and TCRζ. CD28H: amino acids 1-150; NKG2D: amino acids 151-171; 2B4: amino acids 172-291; TCRζ: nucleotides 292-404.

MGSPGMVLGLLVQIWALQEASSLSVQQGPNLLQVRQGSQATLVCQVDQAT
AWERLRVKWTKDGAILCQPYITNGSLSLGVCGPQGRLSWQAPSHLTLQLD
PVSLNHSGAYVCWAAVEIPELEEAEGNITRLFVDPDDPTQNRNRIASFPG
PFFFCCFIAVAMGIRFIIMVTWRRKRKEKQSETSPKEFLTIYEDVKDLKT
RRNHEQEQTFPGGGSTIYSMIQSQSSAPTSQEPAYTLYSLIQPSRKSGSR
KRNHSPSENSTIYEVIGKSQPKAQNPARLSRKELENFDVYSRVKFSRSAD
APAYQQGQNQLYNELNLGRREEYDVLDKRRGRDPEMGGKPQRRKNPQEGL
YNELQKDKMAEAYSEIGMKGERRRGKGHDGLYQGLSTATKDTYDALHMQA
LPPR

SEQ ID NO: 11 is the nucleic acid sequence of CD19 scFv, followed by a CD8α hinge domain fused to the transmembrane domain of CD28H and signaling domains of CD28H, 2B4, and TCRζ. This construct includes an N-terminal Myc tag. DNA sequence of CD8α and CD28H portions were codon-optimized for synthesis as a gBlock fragment. Signal peptide: nucleotides 1-63; Myc tag: nucleotides 64-93; CD19 scFv: nucleotides 94-819; CD8α hinge: 820-954; CD28H transmembrane and signaling domains: nucleotides 955-1350; 2B4: nucleotides 1351-1710; TCRζ: nucleotides 1711-2049.

atggcgctccctgtcaccgcactgcttcttccgctggcactgctgctgc
acgctgcacggcctgagcaaaaacttatctctgaagaggacctcgatat
acagatgacgcagacaacgtcaagtctttccgccagcttgggagaccga
gtgactatatcttgtagagcaagccaggatatttctaagtatcttaact
ggtaccaacaaaagcccgatggaacggttaagctgcttatataccatac
cagtagactccactccggcgtaccatcacggttttctggcagtggctcc
gggaccgactattctttgacgatctctaatctcgaacaagaggatattg
caacatacttttgtcagcaaggcaataccttgccatatacgtttggggg
gggacaaaacttgagataaccggcggcggtggttcaggcggtggcggtt
ccggtggtgggggatcagaggttaagcttcaggaatccggaccaggttt
ggttgcccccagccaatctctcagcgttacatgcacggtttcaggcgtc
agtctccccgattacggtgtaagttggattcggcaacctccgcgaaagg
gtctggaatggctgggggttatttgggggagtgagacaacttattacaa
ctctgcacttaagagtcggcttaccatcatcaaggataattcaaaatca
caagtattcctgaagatgaactcattgcaaacagatgatacagctatat
actattgtgccaagcattactattatggtggttcttatgcaatggatta
ctgggggcaaggcacgtcagtgacagtgagttcaACCACCACCCCTGCA
CCAAGACCTCCAACTCCTGCCCCAACTATTGCAAGTCAGCCACTTTCTT
TGCGACCAGAGGCGTGCCGGCCAGCGGCGGGGGGCGCAGTGCACACGAG
GGGGCTGGACTTCGCCTGTGATTTCCTCTTCGTGCTGCTGGGGGTGGGA
AGCATGGGTGTGGCTGCGATCGTGTGGGGTGCCTGGTTCTGGGGCCGCC
GCAGCTGCCAGCAAAGGGACTCAGGTAACAGCCCAGGAAATGCATTCTA
CAGCAACGTCCTATACCGGCCCCGGGGGGCCCCAAAGAAGAGTGAGGAC
TGCTCTGGAGAGGGGAAGGACCAGAGGGGCCAGAGCATTTATTCAACCA
GTTTCCCTCAACCGGCTCCAAGACAACCACATCTCGCCAGTCGGCCTTG
TCCGTCCCCTAGACCCTGCCCCAGTCCCAGGCCCGGCCACCCCGTCTCT
ATGGTCAGGGTCTCTCCTAGACCAAGCCCCACCCAGCAGCCGAGGCCAA
AAGGGTTCCCCAAAGTGGGAGAGGAGTGGAGGAGAAAGAGGAAGGAGAA
GCAGTCAGAGACCAGTCCCAAGGAATTTTTGACAATTTACGAAGATGTC
AAGGATCTGAAAACCAGGAGAAATCACGAGCAGGAGCAGACTTTTCCTG
GAGGGGGGAGCACCATCTACTCTATGATCCAGTCCCAGTCTTCTGCTCC
CACGTCACAAGAACCTGCATATACATTATATTCATTAATTCAGCCTTCC
AGGAAGTCTGGATCCAGGAAGAGGAACCACAGCCCTTCCTTCAATAGCA
CTATCTATGAAGTGATTGGAAAGAGTCAACCTAAAGCCCAGAACCCTGC
TCGATTGAGCCGCAAAGAGCTGGAGAACTTTGATGTTTATTCCagagtg
aagttcagcaggagcgcagacgcccccgcgtaccagcagggccagaacc
agctctataacgagctcaatctaggacgaagagaggagtacgatgtttt
ggacaagagacgtggccgggaccctgagatggggggaaagccgcagaga
aggaagaaccctcaggaaggcctgtacaatgaactgcagaaagataaga
tggcggaggcctacagtgagattgggatgaaaggcgagcgccggagggg
caaggggcacgatggcctttaccagggtctcagtacagccaccaaggac
acctacgacgcccttcacatgcaggccctgccccctcgc

SEQ ID NO: 12 is the amino acid sequence of CD19 scFv, followed by a CD8α hinge domain fused to the transmembrane domain of CD28H and signaling domains of CD28H, 2B4, and TCRζ. This construct includes an N-terminal Myc tag. Signal peptide: amino acids 1-21; Myc tag: amino acids 22-31; CD19 scFv: amino acids 32-273; CD8α hinge: 274-318; CD28H transmembrane and signaling domains: amino acids 319-450; 2B4: amino acids 451-570; TCRζ: amino acids 571-683.

MALPVTALLLPLALLLHAARPEQKLISEEDLDIQMTQTTSSLSASLGDR
VTISCRASQDISKYLNWYQQKPDGTVKLLIYHTSRLHSGVPSRFSGSGS
GTDYSLTISNLEQEDIATYFCQQGNTLPYTFGGGTKLEITGGGGSGGGG
GGGGSEVKLQESGPGLVAPSQSLSVTCTVSGVSLPDYGVSWIRQPPRKG
LEWLGVIWGSETTYYNSALKSRLTIIKDNSKSQVFLKMNSLQTDDTAIY
YCAKHYYYGGSYAMDYWGQGTSVTVSSTTTPAPRPPTPAPTIASQPLSL
RPEACRPAAGGAVHTRGLDFACDELFVLLGVGSMGVAAIVWGAWFWGRR
SCQQRDSGNSPGNAFYSNVLYRPRGAPKKSEDCSGEGKDQRGQSIYSTS
FPQPAPRQPHLASRPCPSPRPCPSPRPGHPVSMVRVSPRPSPTQQPRPK
GFPKVGEEWRRKRKEKQSETSPKEFLTIYEDVKDLKTRRNHEQEQTFPG
GGSTIYSMIQSQSSAPTSQEPAYTLYSLIQPSRKSGSRKRNHSPSENST
IYEVIGKSQPKAQNPARLSRKELENFDVYSRVKFSRSADAPAYQQGQNQ
LYNELNLGRREEYDVLDKRRGRDPEMGGKPQRRKNPQEGLYNELQKDKM
AEAYSEIGMKGERRRGKGHDGLYQGLSTATKDTYDALHMQALPPR

SEQ ID NO: 13 is the nucleic acid sequence of CD19 scFv, followed by a CD8α hinge domain fused to the transmembrane domain of CD28 and signaling domains of CD28, 4-1BB, and TCRζ. This construct includes an N-terminal Myc tag. DNA sequence of CD8α was codon-optimized for synthesis as a gBlock fragment. Signal peptide: nucleotides 1-63; Myc tag: nucleotides 64-93; CD19 scFv: nucleotides 94-819; CD8α hinge: nucleotides 820-954; CD28 transmembrane and signaling domains: nucleotides 955-1158; 4-1BB: nucleotides 1159-1284; TCRζ: nucleotides 1285-1623.

atggcgctccctgtcaccgcactgcttcttccgctggcactgctgctgc
acgctgcacggcctgagcaaaaacttatctctgaagaggacctcgatat
acagatgacgcagacaacgtcaagtctttccgccagcttgggagaccga
gtgactatatcttgtagagcaagccaggatatttctaagtatcttaact
ggtaccaacaaaagcccgatggaacggttaagctgcttatataccatac
cagtagactccactccggcgtaccatcacggttttctggcagtggctcc
gggaccgactattctttgacgatctctaatctcgaacaagaggatattg
caacatacttttgtcagcaaggcaataccttgccatatacgtttggggg
gggacaaaacttgagataaccggcggcggtggttcaggcggtggcggtt
ccggtggtgggggatcagaggttaagcttcaggaatccggaccaggttt
ggttgcccccagccaatctctcagcgttacatgcacggtttcaggcgtc
agtctccccgattacggtgtaagttggattcggcaacctccgcgaaagg
gtctggaatggctgggggttatttgggggagtgagacaacttattacaa
ctctgcacttaagagtcggcttaccatcatcaaggataattcaaaatca
caagtattcctgaagatgaactcattgcaaacagatgatacagctatat
actattgtgccaagcattactattatggtggttcttatgcaatggatta
ctgggggcaaggcacgtcagtgacagtgagttcaACCACCACCCCTGCA
CCAAGACCTCCAACTCCTGCCCCAACTATTGCAAGTCAGCCACITTCTT
TGCGACCAGAGGCGTGCCGGCCAGCGGCGGGGGGCGCAGTGCACACGAG
GGGGCTGGACTTCGCCTGTGATTTTTGGGTGCTGGTGGTGGTTGGTGGA
GTCCTGGCTTGCTATAGCTTGCTAGTAACAGTGGCCTTTATTATTTTCT
GGGTGAGGAGTAAGAGGAGCAGGCTCCTGCACAGTGACTACATGAACAT
GACTCCCCGCCGCCCCGGGCCCACCCGCAAGCATTACCAGCCCTATGCC
CCACCACGCGACTTCGCAGCCTATCGCTCCAAACGGGGCAGAAAGAAAC
TCCTGTATATATTCAAACAACCATTTATGAGACCAGTACAAACTACTCA
AGAGGAAGATGGCTGTAGCTGCCGATTTCCAGAAGAAGAAGAAGGAGGA
TGTGAACTGagagtgaagttcagcaggagcgcagacgcccccgcgtacc
agcagggccagaaccagctctataacgagctcaatctaggacgaagaga
ggagtacgatgttttggacaagagacgtggccgggaccctgagatgggg
ggaaagccgcagagaaggaagaaccctcaggaaggcctgtacaatgaac
tgcagaaagataagatggcggaggcctacagtgagattgggatgaaagg
cgagcgccggaggggcaaggggcacgatggcctttaccagggtctcagt
acagccaccaaggacacctacgacgcccttcacatgcaggccctgcccc
ctcgc

SEQ ID NO: 14 is the amino acid sequence of CD19 scFv, followed by a CD8α hinge domain fused to the transmembrane domain of CD28 and signaling domains of CD28, 4-1BB, and TCRζ. This construct includes an N-terminal Myc tag. Signal peptide: amino acids 1-21; Myc tag: amino acids 22-31; CD19 scFv: amino acids 32-273; CD8α hinge: amino acids 274-318; CD28 transmembrane and signaling domains: amino acids 319-386; 4-1BB: amino acids 387-428; TCRζ: amino acids 429-541.

MALPVTALLLPLALLLHAARPEQKLISEEDLDIQMTQTTSSLSASLGDR
VTISCRASQDISKYLNWYQQKPDGTVKLLIYHTSRLHSGVPSRFSGSGS
GTDYSLTISNLEQEDIATYFCQQGNTLPYTFGGGTKLEITGGGGSGGGG
SGGGGSEVKLQESGPGLVAPSQSLSVTCTVSGVSLPDYGVSWIRQPPRK
GLEWLGVIWGSETTYYNSALKSRLTIIKDNSKSQVFLKMNSLQTDDTAI
YYCAKHYYYGGSYAMDYWGQGTSVTVSSTTTPAPRPPTPAPTIASQPLS
LRPEACRPAAGGAVHTRGLDFACDEWVLVVVGGVLACYSLLVTVAFIIF
WVRSKRSRLLHSDYMNMTPRRPGPTRKHYQPYAPPRDFAAYRSKRGRKK
LLYIFKQPFMRPVQTTQEEDGCSCRFPEEEEGGCELRVKFSRSADAPAY
QQGQNQLYNELNLGRREEYDVLDKRRGRDPEMGGKPQRRKNPQEGLYNE
LQKDKMAEAYSEIGMKGERRRGKGHDGLYQGLSTATKDTYDALHMQALP
PR

SEQ ID NO: 15 is the nucleic acid sequence of CD19 scFv, followed by a CD8α hinge domain fused to the transmembrane domain of CD28H and signaling domains of CD28H and TCRζ. This construct includes an N-terminal Myc tag. DNA sequence of CD8α and CD28H portions were codon-optimized for synthesis as a gBlock fragment. Signal peptide: nucleotides 1-63; Myc tag: nucleotides 64-93; CD19 scFv: nucleotides 94-819; CD8α hinge: nucleotides 820-954; CD28H transmembrane domain: nucleotides 955-1017; CD28H signaling domain: nucleotides 1018-1350; TCRζ: nucleotides 1351-1689.

atggcgctccctgtcaccgcactgcttcttccgctggcactgctgctgc
acgctgcacggcctgagcaaaaacttatctctgaagaggacctcgatat
acagatgacgcagacaacgtcaagtctttccgccagcttgggagaccga
gtgactatatcttgtagagcaagccaggatatttctaagtatcttaact
ggtaccaacaaaagcccgatggaacggttaagctgcttatataccatac
cagtagactccactccggcgtaccatcacggttttctggcagtggctcc
gggaccgactattctttgacgatctctaatctcgaacaagaggatattg
caacatacttttgtcagcaaggcaataccttgccatatacgtttggggg
gggacaaaacttgagataaccggcggcggtggttcaggcggtggcggtt
ccggtggtgggggatcagaggttaagcttcaggaatccggaccaggttt
ggttgcccccagccaatctctcagcgttacatgcacggtttcaggcgtc
agtctccccgattacggtgtaagttggattcggcaacctccgcgaaagg
gtctggaatggctgggggttatttgggggagtgagacaacttattacaa
ctctgcacttaagagtcggcttaccatcatcaaggataattcaaaatca
caagtattcctgaagatgaactcattgcaaacagatgatacagctatat
actattgtgccaagcattactattatggtggttcttatgcaatggatta
ctgggggcaaggcacgtcagtgacagtgagttcaACCACCACCCCTGCA
CCAAGACCTCCAACTCCTGCCCCAACTATTGCAAGTCAGCCACTTTCTT
TGCGACCAGAGGCGTGCCGGCCAGCGGCGGGGGGCGCAGTGCACACGAG
GGGGCTGGACTTCGCCTGTGATTTCCTCTTCGTGCTGCTGGGGGTGGGA
AGCATGGGTGTGGCTGCGATCGTGTGGGGTGCCTGGTTCTGGGGCCGCC
GCAGCTGCCAGCAAAGGGACTCAGGTAACAGCCCAGGAAATGCATTCTA
CAGCAACGTCCTATACCGGCCCCGGGGGGCCCCAAAGAAGAGTGAGGAC
TGCTCTGGAGAGGGGAAGGACCAGAGGGGCCAGAGCATTTATTCAACCA
GTTTCCCTCAACCGGCTCCAAGACAACCACATCTCGCCAGTCGGCCTTG
TCCGTCCCCTAGACCCTGCCCCAGTCCCAGGCCCGGCCACCCCGTCTCT
ATGGTCAGGGTCTCTCCTAGACCAAGCCCCACCCAGCAGCCGAGGCCAA
AAGGGTTCCCCAAAGTGGGAGAGGAGagagtgaagttcagcaggagcgc
agacgcccccgcgtaccagcagggccagaaccagctctataacgagctc
aatctaggacgaagagaggagtacgatgttttggacaagagacgtggcc
gggaccctgagatggggggaaagccgcagagaaggaagaaccctcagga
aggcctgtacaatgaactgcagaaagataagatggcggaggcctacagt
gagattgggatgaaaggcgagcgccggaggggcaaggggcacgatggcc
tttaccagggtctcagtacagccaccaaggacacctacgacgcccttca
catgcaggccctgccccctcgc

SEQ ID NO: 16 is the amino acid sequence of CD19 scFv, followed by a CD8α hinge domain fused to the transmembrane domain of CD28H and signaling domains of CD28H and TCRζ. This construct includes an N-terminal Myc tag. Signal peptide: amino acids 1-21; Myc tag: amino acids 22-31; CD19 scFv: amino acids 32-273; CD8α hinge: amino acids 274-318; CD28H transmembrane domain: amino acids 319-339; CD28H signaling domain: amino acids 340-450; TCRζ: amino acids 451-563.

MALPVTALLLPLALLLHAARPEQKLISEEDLDIQMTQTTSSLSASLGDR
VTISCRASQDISKYLNWYQQKPDGTVKLLIYHTSRLHSGVPSRFSGSGS
GTDYSLTISNLEQEDIATYFCQQGNTLPYTFGGGTKLEITGGGGSGGGG
SGGGGSEVKLQESGPGLVAPSQSLSVTCTVSGVSLPDYGVSWIRQPPRK
GLEWLGVIWGSETTYYNSALKSRLTIIKDNSKSQVFLKMNSLQTDDTAI
YYCAKHYYYGGSYAMDYWGQGTSVTVSSTTTPAPRPPTPAPTIASQPLS
LRPEACRPAAGGAVHTRGLDFACDELFVLLGVGSMGVAAIVWGAWFWGR
RSCQQRDSGNSPGNAFYSNVLYRPRGAPKKSEDCSGEGKDQRGQSIYST
SFPQPAPRQPHLASRPCPSPRPCPSPRPGHPVSMVRVSPRPSPTQQPRP
KGFPKVGEERVKFSRSADAPAYQQGQNQLYNELNLGRREEYDVLDKRRG
RDPEMGGKPQRRKNPQEGLYNELQKDKMAEAYSEIGMKGERRRGKGHDG
LYQGLSTATKDTYDALHMQALPPR

SEQ ID NO: 17 is the nucleic acid sequence of CD19 scFv, followed by a CD8α hinge domain fused to the transmembrane domain of CD28 and signaling domains of CD28 and TCRζ. This construct includes an N-terminal Myc tag. DNA sequence of CD8α was codon-optimized for synthesis as a gBlock fragment. Signal peptide: nucleotides 1-63; Myc tag: nucleotides 64-93; CD19 scFv: nucleotides 94-819; CD8α hinge: nucleotides 820-954; CD28 transmembrane domain: nucleotides 955-1035; CD28 signaling domain: nucleotides 1036-1158; TCRζ: nucleotides 1159-1497.

atggcgctccctgtcaccgcactgcttcttccgctggcactgctgctgc
acgctgcacggcctgagcaaaaacttatctctgaagaggacctcgatat
acagatgacgcagacaacgtcaagtctttccgccagcttgggagaccga
gtgactatatcttgtagagcaagccaggatatttctaagtatcttaact
ggtaccaacaaaagcccgatggaacggttaagctgcttatataccatac
cagtagactccactccggcgtaccatcacggttttctggcagtggctcc
gggaccgactattctttgacgatctctaatctcgaacaagaggatattg
caacatacttttgtcagcaaggcaataccttgccatatacgtttggggg
gggacaaaacttgagataaccggcggcggtggttcaggcggtggcggtt
ccggtggtgggggatcagaggttaagcttcaggaatccggaccaggttt
ggttgcccccagccaatctctcagcgttacatgcacggtttcaggcgtc
agtctccccgattacggtgtaagttggattcggcaacctccgcgaaagg
gtctggaatggctgggggttatttgggggagtgagacaacttattacaa
ctctgcacttaagagtcggcttaccatcatcaaggataattcaaaatca
caagtattcctgaagatgaactcattgcaaacagatgatacagctatat
actattgtgccaagcattactattatggtggttcttatgcaatggatta
ctgggggcaaggcacgtcagtgacagtgagttcaACCACCACCCCTGCA
CCAAGACCTCCAACTCCTGCCCCAACTATTGCAAGTCAGCCACTTTCTT
TGCGACCAGAGGCGTGCCGGCCAGCGGCGGGGGGCGCAGTGCACACGAG
GGGGCTGGACTTCGCCTGTGATTTTTGGGTGCTGGTGGTGGTTGGTGGA
GTCCTGGCTTGCTATAGCTTGCTAGTAACAGTGGCCTTTATTATTTTCT
GGGTGAGGAGTAAGAGGAGCAGGCTCCTGCACAGTGACTACATGAACAT
GACTCCCCGCCGCCCCGGGCCCACCCGCAAGCATTACCAGCCCTATGCC
CCACCACGCGACTTCGCAGCCTATCGCTCCagagtgaagttcagcagga
gcgcagacgcccccgcgtaccagcagggccagaaccagctctataacga
gctcaatctaggacgaagagaggagtacgatgttttggacaagagacgt
ggccgggaccctgagatggggggaaagccgcagagaaggaagaaccctc
aggaaggcctgtacaatgaactgcagaaagataagatggcggaggccta
cagtgagattgggatgaaaggcgagcgccggaggggcaaggggcacgat
ggcctttaccagggtctcagtacagccaccaaggacacctacgacgccc
ttcacatgcaggccctgccccctcgc

SEQ ID NO: 18 is the amino acid sequence of CD19 scFv, followed by a CD8α hinge domain fused to the transmembrane domain of CD28 and signaling domains of CD28 and TCRζ. This construct includes an N-terminal Myc tag. Signal peptide: amino acids 1-21; Myc tag: amino acids 22-31; CD19 scFv: amino acids 32-273; CD8α hinge: amino acids 274-318; CD28 transmembrane domain: amino acids 319-345; CD28 signaling domain: amino acids 346-386; TCRζ: amino acids 387-499.

MALPVTALLLPLALLLHAARPEQKLISEEDLDIQMTQTTSSLSASLGDR
VTISCRASQDISKYLNWYQQKPDGTVKLLIYHTSRLHSGVPSRFSGSGS
GTDYSLIISNLEQEDIATYFCQQGNTLPYTFGGGTKLEITGGGGSGGGG
SGGGGSEVKLQESGPGLVAPSQSLSVTCTVSGVSLPDYGVSWIRQPPRK
GLEWLGVIWGSETTYYNSALKSRLTIIKDNSKSQVFLKMNSLQTDDTAI
YYCAKHYYYGGSYAMDYWGQGTSVTVSSTTTPAPRPPTPAPTIASQPLS
LRPEACRPAAGGAVHTRGLDFACDFWVLVVVGGVLACYSLLVTVAFIIF
WVRSKRSRLLHSDYMNMTPRRPGPTRKHYQPYAPPRDFAAYRSRVKFSR
SADAPAYQQGQNQLYNELNLGRREEYDVLDKRRGRDPEMGGKPQRRKNP
QEGLYNELQKDKMAEAYSEIGMKGERRRGKGHDGLYQGLSTATKDTYDA
LHMQALPPR

SEQ ID NO: 19 is the nucleic acid sequence of CD19 scFv, followed by a CD8α hinge domain fused to the transmembrane domain of CD28H and signaling domain of TCRζ. This construct includes an N-terminal Myc tag. DNA sequence of CD8α was codon-optimized for synthesis as a gBlock fragment. Signal peptide: nucleotides 1-63; Myc tag: nucleotides 64-93; CD19 scFv: nucleotides 94-819; CD8α hinge: nucleotides 820-954; CD28H transmembrane domain: nucleotides 955-1017; TCRζ: nucleotides 1018-1356.

atggcgctccctgtcaccgcactgcttcttccgctggcactgctgctgc
acgctgcacggcctgagcaaaaacttatctctgaagaggacctcgatat
acagatgacgcagacaacgtcaagtctttccgccagcttgggagaccga
gtgactatatcttgtagagcaagccaggatatttctaagtatcttaact
ggtaccaacaaaagcccgatggaacggttaagctgcttatataccatac
cagtagactccactccggcgtaccatcacggttttctggcagtggctcc
gggaccgactattctttgacgatctctaatctcgaacaagaggatattg
caacatacttttgtcagcaaggcaataccttgccatatacgtttggggg
gggacaaaacttgagataaccggcggcggtggttcaggcggtggcggtt
ccggtggtgggggatcagaggttaagcttcaggaatccggaccaggttt
ggttgcccccagccaatctctcagcgttacatgcacggtttcaggcgtc
agtctccccgattacggtgtaagttggattcggcaacctccgcgaaagg
gtctggaatggctgggggttatttgggggagtgagacaacttattacaa
ctctgcacttaagagtcggcttaccatcatcaaggataattcaaaatca
caagtattcctgaagatgaactcattgcaaacagatgatacagctatat
actattgtgccaagcattactattatggtggttcttatgcaatggatta
ctgggggcaaggcacgtcagtgacagtgagttcaACCACCACCCCTGCA
CCAAGACCTCCAACTCCTGCCCCAACTATTGCAAGTCAGCCACTTTCTT
TGCGACCAGAGGCGTGCCGGCCAGCGGCGGGGGGCGCAGTGCACACGAG
GGGGCTGGACTTCGCCTGTGATTTCCTCTTCGTGCTGCTGGGGGTGGGA
AGCATGGGTGTGGCTGCGATCGTGTGGGGTGCCTGGagagtgaagttca
gcaggagcgcagacgcccccgcgtaccagcagggccagaaccagctcta
taacgagctcaatctaggacgaagagaggagtacgatgttttggacaag
agacgtggccgggaccctgagatggggggaaagccgcagagaaggaaga
accctcaggaaggcctgtacaatgaactgcagaaagataagatggcgga
ggcctacagtgagattgggatgaaaggcgagcgccggaggggcaagggg
cacgatggcctttaccagggtctcagtacagccaccaaggacacctacg
acgcccttcacatgcaggccctgccccctcgc

SEQ ID NO: 20 is the amino acid sequence of CD19 scFv, followed by a CD8α hinge domain fused to the transmembrane domain of CD28H and signaling domain of TCRζ. This construct includes an N-terminal Myc tag. Signal peptide: amino acids 1-21; Myc tag: amino acids 22-31; CD19 scFv: amino acids 32-273; CD8α hinge: amino acids 274-318; CD28H transmembrane domain: amino acids 319-339; TCRζ: amino acids 340-452.

MALPVTALLLPLALLLHAARPEQKLISEEDLDIQMTQTTSSLSASLGDR
VTISCRASQDISKYLNWYQQKPDGTVKLLIYHTSRLHSGVPSRFSGSGS
GTDYSLIISNLEQEDIATYFCQQGNTLPYTFGGGTKLEITGGGGSGGGG
SGGGGSEVKLQESGPGLVAPSQSLSVTCTVSGVSLPDYGVSWIRQPPRK
GLEWLGVIWGSETTYYNSALKSRLTIIKDNSKSQVFLKMNSLQTDDTAI
YYCAKHYYYGGSYAMDYWGQGTSVTVSSTTTPAPRPPTPAPTIASQPLS
LRPEACRPAAGGAVHTRGLDFACDELFVLLGVGSMGVAAIVWGAWRVKF
SRSADAPAYQQGQNQLYNELNLGRREEYDVLDKRRGRDPEMGGKPQRRK
NPQEGLYNELQKDKMAEAYSEIGMKGERRRGKGHDGLYQGLSTATKDTY
DALHMQALPPR

SEQ ID NO: 21 is an exemplary amino acid sequence of CD16 (FCGR3A)

MWQLLLPTALLLLVSAGMRTEDLPKAVVFLEPQWYRVLEKDSVTLKCQG
AYSPEDNSTQWFHNESLISSQASSYFIDAATVDDSGEYRCQTNLSTLSD
PVQLEVHIGWLLLQAPRWVFKEEDPIHLRCHSWKNTALHKVTYLQNGKG
RKYFHHNSDFYIPKATLKDSGSYFCRGLFGSKNVSSETVNITITQGLAV
STISSFFPPGYQVSFCLVMVLLFAVDIGLYFSVKTNIRSSTRDWKDHKF
KWRKDPQDK

SEQ ID NO: 22 is an exemplary amino acid sequence of NKp46

MSSTLPALLCVGLCLSQRISAQQQTLPKPFIWAEPHFMVPKEKQVTICC
QGNYGAVEYQLHFEGSLFAVDRPKPPERINKVKFYIPDMNSRMAGQYSC
IYRVGELWSEPSNLLDLVVTEMYDTPTLSVHPGPEVISGEKVTFYCRLD
TATSMELLLKEGRSSHVQRGYGKVQAEFPLGPVTTAHRGTYRCFGSYNN
HAWSFPSEPVKLLVTGDIENTSLAPEDPTFPADTWGTYLLTTETGLQKD
HALWDHTAQNLLRMGLAFLVLVALVWFLVEDWLSRKRTRERASRASTWE
GRRRLNTQTL

SEQ ID NO: 23 is an exemplary amino acid sequence of NKp30

MAWMLLLILIMVHPGSCALWVSQPPEIRTLEGSSAFLPCSFNASQGRLA
IGSVTWERDEVVPGKEVRNGTPEFRGRLAPLASSRFLHDHQAELHIRDV
RGHDASIYVCRVEVLGLGVGTGNGTRLVVEKEHPQLGAGTVLLLRAGFY
AVSFLSVAVGSTVYYQGKCLTWKGPRRQLPAVVPAPLPPPCGSSAHLLP
PVPGG

SEQ ID NO: 24 is an exemplary amino acid sequence of NKp44

MAWRALHPLLLLLLLFPGSQAQSKAQVLQSVAGQTLTVRCQYPPTGSLY
EKKGWCKEASALVCIRLVTSSKPRIMAWTSRFTIWDDPDAGFFTVTMTD
LREEDSGHYWCRIYRPSDNSVSKSVRFYLVVSPASASTQTSWTPRDLVS
SQTQTQSCVPPTAGARQAPESPSTIPVPSQPQNSTLRPGPAAPIALVPV
FCGLLVAKSLVLSALLVWWGDIWWKIMMELRSLDTQKATCHLQQVTDLP
WTSVSSPVEREILYHTVARTKISDDDDEHTL

SEQ ID NO: 25 is an exemplary amino acid sequence of KIR2DS4

MSLMVIIMACVGFFLLQGAWPQEGVHRKPSFLALPGHLVKSEETVILQC
WSDVMFEHFLLHREGKENNTLHLIGEHHDGVSKANFSIGPMMPVLAGTY
RCYGSVPHSPYQLSAPSDPLDMVIIGLYEKPSLSAQPGPTVQAGENVTL
SCSSRSSYDMYHLSREGEAHERRLPAVRSINGTFQADFPLGPATHGGTY
RCFGSFRDAPYEWSNSSDPLLVSVTGNPSNSWPSPTEPSSKTGNPRHLH
VLIGTSVVKIPFTILLFELLHRWCSDKKNAAVMDQEPAGNRTVNSEDSD
EQDHQEVSYA

### DETAILED DESCRIPTION

As demonstrated herein, CARs including an intracellular signaling domain from CD28H provide increased anti-tumor activity and ability to overcome inhibition. In particular examples, a combination of intracellular signaling domains from CD28H and 2B4 were used. As described in the Examples, compared to a third-generation T-cell CAR (CD28-41BB-TCRζ), a CAR including domains from co-activation receptors CD28H and 2B4 (CD244) (CD28H-2B4-TCRζ) induced stronger anti-tumor cytotoxicity by NK cells and was more potent in its ability to overcome inhibition by CD94-NKG2A and KIR2DL1. Similarly, a CAR including co-activation receptors CD28H and 2B4 (CD28H-2B4-TCRζ) induced stronger anti-tumor cytotoxicity by NK cells and was more potent in its ability to overcome inhibition by CD94-NKG2A and KIR2DL1 than another CAR composed of the transmembrane domain of NKG2D and the intracellular signaling domains from 2B4 and TCRζ(NKG2D-2B4-TCRζ).

### I. Terms

Unless otherwise noted, technical terms are used according to conventional usage. Definitions of common terms in molecular biology may be found in Lewin's Genes X, ed. Krebs et al., Jones and Bartlett Publishers, 2009 (ISBN 0763766321); Kendrew et al. (eds.), The Encyclopedia of Molecular Biology, published by Blackwell Publishers, 1994 (ISBN 0632021829); Robert A. Meyers (ed.), Molecular Biology and Biotechnology: a Comprehensive Desk Reference, published by Wiley, John & Sons, Inc., 1995 (ISBN 0471186341); and George P. Rédei, Encyclopedic Dictionary of Genetics. Genomics, Proteomics and Informatics, 3^{rd} Edition, Springer, 2008 (ISBN: 1402067534), and other similar references.

Unless otherwise explained, all technical and scientific terms used herein have the same meaning as commonly understood by one of ordinary skill in the art to which this disclosure belongs. The singular terms “a,” “an,” and “the” include plural referents unless the context clearly indicates otherwise. “Comprising A or B” means including A, or B, or A and B. It is further to be understood that all base sizes or amino acid sizes, and all molecular weight or molecular mass values, given for nucleic acids or polypeptides are approximate, and are provided for description.

Although methods and materials similar or equivalent to those described herein can be used in the practice or testing of the present disclosure, suitable methods and materials are described below. All publications, patent applications, patents, and other references mentioned herein are incorporated by reference in their entirety, as are the GenBank Accession numbers (for the sequences present in GenBank on Mar. 26, 2020). In case of conflict, the present specification, including explanations of terms, will control. In addition, the materials, methods, and examples are illustrative only and not intended to be limiting.

In order to facilitate review of the various embodiments of the disclosure, the following explanations of specific terms are provided:

Antibody: A polypeptide ligand comprising at least one variable region that recognizes and binds (such as specifically recognizes and specifically binds) an epitope of an antigen. Mammalian immunoglobulin molecules are composed of a heavy (H) chain and a light (L) chain, each of which has a variable region, termed the variable heavy (V_{H}) region and the variable light (V_{L}) region, respectively. Together, the V_{H} region and the V_{L} region are responsible for binding the antigen recognized by the antibody. There are five main heavy chain classes (or isotypes) of mammalian immunoglobulin, which determine the functional activity of an antibody molecule: IgM, IgD, IgG, IgA and IgE.

Antibody variable regions contain “framework” regions and hypervariable regions, known as “complementarity determining regions” or “CDRs.” The CDRs are primarily responsible for binding to an epitope of an antigen. The framework regions of an antibody serve to position and align the CDRs in three-dimensional space. The amino acid sequence boundaries of a given CDR can be readily determined using any of a number of well-known numbering schemes, including those described by Kabat et al. (Sequences of Proteins of Immunological Interest, U.S. Department of Health and Human Services, 1991; the “Kabat” numbering scheme), Chothia et al. (see Chothia and Lesk, J Mol Biol 196:901-917, 1987; Chothia et al., Nature 342:877, 1989; and Al-Lazikani et al., (JMB 273,927-948, 1997; the “Chothia” numbering scheme), and the ImMunoGeneTics (IMGT) database (see, Lefranc, Nucleic Acids Res 29:207-9, 2001; the “IMGT” numbering scheme). The Kabat and IMGT databases are maintained online.

A single-chain antibody (scFv) is a genetically engineered molecule containing the V_{H} and V_{L} domains of one or more antibody(ies) linked by a suitable polypeptide linker as a genetically fused single chain molecule (see, for example, Bird et al., Science, 242:423-426, 1988; Huston et al., Proc. Natl. Acad. Sci., 85:5879-5883, 1988; Ahmad et al., Clin. Dev. Immunol., 2012, doi:10.1155/2012/980250; Marbry, IDrugs, 13:543-549, 2010). The intramolecular orientation of the V_{H}-domain and the V_{L}-domain in a scFv, is typically not decisive for scFvs. Thus, scFvs with both possible arrangements (V_{H}-domain-linker domain-V_{L}-domain; V_{L}-domain-linker domain-V_{H}-domain) may be used. In a dsFv the V_{H} and V_{L} have been mutated to introduce a disulfide bond to stabilize the association of the chains. Diabodies also are included, which are bivalent, bispecific antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites (see, for example, Holliger et al., Proc. Natl. Acad. Sci., 90:6444-6448, 1993; Poljak et al., Structure, 2:1121-1123, 1994).

Antibodies also include genetically engineered forms such as chimeric antibodies (such as humanized murine antibodies) and heteroconjugate antibodies (such as bispecific antibodies). See also, Pierce Catalog and Handbook, 1994-1995 (Pierce Chemical Co., Rockford, IL); Kuby, J., Immunology, 3^{rd} Ed., W.H. Freeman & Co., New York, 1997.

2B4 (CD244): An NK cell surface receptor that mediates non-MHC restricted killing. 2B4 is also found on some T cells, dendritic cells, and monocytes. The ligand for 2B4 is CD48, which is expressed on hematopoietic cells. Two human isoforms have been identified. These have identical intracellular domains and differ by the presence or absence of five amino acids in the extracellular domain.

2B4 sequences are publicly available. Exemplary human 2B4 nucleic acid sequences include NM_016382 and NM_001166664. Exemplary human 2B4 amino acid sequences include GenBank Accession Nos. NP_057466 and NP_001160136. One of ordinary skill in the art can identify additional 2B4 nucleic acid and amino acid sequences, for example, utilizing currently available or subsequently developed sequence search and database tools.

CD28 homolog (CD28H): CD28H belongs to the CD28 family of immune receptors, which includes regulators of the immune system, such as checkpoint inhibitors PD-1 and CTLA-4. CD28H was initially described as a molecule involved in cell-cell interaction, cell migration, and angiogenesis of epithelial and endothelial cells. CD28H has a single extracellular immunoglobulin domain followed by a transmembrane domain and a 110 amino acid-long cytoplasmic region. CD28H is a costimulatory receptor in naïve T cells. The ligand of CD28H is B7 homolog 7 (B7H7, also known as B7-H5, encoded by the gene HHL42), which co-stimulates T-cell growth and cytokine production. Besides expression on antigen presenting cells after stimulation, B7H7 is also broadly expressed in tumor tissues. CD28H⁺ naïve and memory T cells show diminished effector function and increased naïve features. CD28H expression has been described on NK cells, innate lymphoid cells (ILCs), and plasmacytoid dendritic cells (pDC) in human peripheral blood.

CD28H sequences are publicly available. Exemplary human CD28H nucleic acid sequences include NM_144615 and NM_001308232. Exemplary human CD28H amino acid sequences include GenBank Accession Nos. NP_653216 and NP_001295161. One of ordinary skill in the art can identify additional CD28H nucleic acid and amino acid sequences, for example, utilizing currently available or subsequently developed sequence search and database tools.

Chimeric antigen receptor (CAR): A chimeric molecule that includes an target-binding portion (such as a single domain antibody or scFv) and a signaling domain, such as a signaling domain from a T cell receptor (e.g. CD3┌, also referred to herein as “TCRζ”). Typically, CARs include a target-binding portion, a transmembrane domain, and an intracellular domain. The intracellular domain typically includes a signaling domain having one or more immunoreceptor tyrosine-based activation motifs (ITAM), such as TCRζ (CD3⁻Π or Fc [symbol] R1 [symbol] . In some instances, the intracellular domain also includes the intracellular portion of at least one additional co-stimulatory domain or intracellular signaling molecule.

DAP12 (TYROBP): A transmembrane protein including an ITAM motif in the cytoplasmic domain, which may act as an activating signal transduction element and may bind ZAP70 and SYK.

DAP12 sequences are publicly available. Exemplary human DAP12 nucleic acid sequences include NM_198125 and NM_001173515. Exemplary human DAP12 amino acid sequences include GenBank Accession Nos. NP_937758 and NP_001166986. One of ordinary skill in the art can identify additional DAP12 nucleic acid and amino acid sequences, for example, utilizing currently available or subsequently developed sequence search and database tools.

Isolated: An “isolated” biological component, such as a nucleic acid, protein (including antibodies) or organelle, has been substantially separated or purified away from other biological components in the environment (such as a cell) in which the component naturally occurs, e.g., other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids.

Natural Killer (NK) cells: Cells of the immune system that kill target cells in the absence of a specific antigenic stimulus and without restriction according to MHC class. Target cells can be tumor cells or cells harboring viruses. NK cells are characterized by the presence of CD56 and the absence of CD3 surface markers. NK cells typically comprise approximately 10-15% of the mononuclear cell fraction in normal peripheral blood. Historically, NK cells were first identified by their ability to lyse certain tumor cells without prior immunization or activation. NK cells are thought to provide a “back up” protective mechanism against viruses and tumors that might escape the CTL response by down-regulating MHC class I presentation. In addition to being involved in direct cytotoxic killing, NK cells also serve a role in cytokine production, which can be important to control cancer and infection.

In some examples, a “modified NK cell” is a NK cell transduced or transformed with a heterologous nucleic acid (such as one or more of the nucleic acids or vectors disclosed herein) or expressing one or more heterologous proteins. The terms “modified NK cell” and “transduced NK cell” are used interchangeably in some examples herein.

Purified: The term purified does not require absolute purity; rather, it is intended as a relative term. Thus, for example, a purified protein or nucleic acid preparation is one in which the protein or nucleic acid is more enriched than the protein or nucleic acid is in its natural environment (e.g., within a cell). In one embodiment, a preparation is purified such that the protein or nucleic acid represents at least 50% of the total protein or nucleic acid content of the preparation. Substantial purification denotes purification from other proteins or cellular components. A substantially purified protein or nucleic acid is at least 60%, 70%, 80%, 90%, 95% or 98% pure. Thus, in one specific, non-limiting example, a substantially purified protein or nucleic acid is 90% free of other components.

Recombinant: A nucleic acid or protein that has a sequence that is not naturally occurring or has a sequence that is made by an artificial combination of two otherwise separated segments of sequence (e.g., a “chimeric” sequence). This artificial combination can be accomplished by chemical synthesis or by the artificial manipulation of isolated segments of nucleic acids, for example, by genetic engineering techniques.

Subject: A living multi-cellular vertebrate organism, a category that includes both human and veterinary subjects, including human and non-human mammals.

T cell: A white blood cell (lymphocyte) that is an important mediator of the immune response. T cells include, but are not limited to, CD4⁺ T cells and CD8⁺ T cells. A CD4⁺ T lymphocyte is an immune cell that carries a marker on its surface known as “cluster of differentiation 4” (CD4). These cells, also known as helper T cells, help orchestrate the immune response, including antibody responses as well as killer T cell responses. CD8⁺ T cells carry the “cluster of differentiation 8” (CD8) marker. In one embodiment, a CD8⁺ T cell is a cytotoxic T lymphocyte (CTL). In another embodiment, a CD8⁺ T cell is a suppressor T cell.

Activated T cells can be detected by an increase in cell proliferation and/or expression of or secretion of one or more cytokines (such as IL-2, IL-4, IL-6, IFNγ, or TNFα). Activation of CD8⁺ T cells can also be detected by an increase in cytolytic activity in response to an antigen.

In some examples, a “modified T cell” is a T cell transduced or transformed with a heterologous nucleic acid (such as one or more of the nucleic acids or vectors disclosed herein) or expressing one or more heterologous proteins. The terms “modified T cell” and “transduced T cell” are used interchangeably in some examples herein.

Transduced or Transformed: A transformed cell is a cell into which a nucleic acid molecule has been introduced by molecular biology techniques. As used herein, the terms transduction and transformation encompass all techniques by which a nucleic acid molecule might be introduced into such a cell, including transfection with viral vectors, the use of plasmid vectors, and introduction of DNA by electroporation, lipofection, and particle gun acceleration.

Treating or ameliorating a disease: “Treating” refers to a therapeutic intervention that decreases or inhibits a sign or symptom of a disease or pathological condition after it has begun to develop, such as a reduction in tumor size or tumor burden. “Ameliorating” refers to the reduction in the number or severity of signs or symptoms of a disease, such as cancer.

Vector: A nucleic acid molecule that can be introduced into a host cell (for example, by transfection or transduction), thereby producing a transformed host cell. Recombinant DNA vectors are vectors having recombinant DNA. A vector can include nucleic acid sequences that permit it to replicate in a host cell, such as an origin of replication. A vector can also include one or more selectable marker genes and other genetic elements known in the art. Viral vectors are recombinant nucleic acid vectors having at least some nucleic acid sequences derived from one or more viruses. A replication deficient viral vector is a vector that requires complementation of one or more regions of the viral genome required for replication due to a deficiency in at least one replication-essential gene function.

### H. Chimeric Antigen Receptors

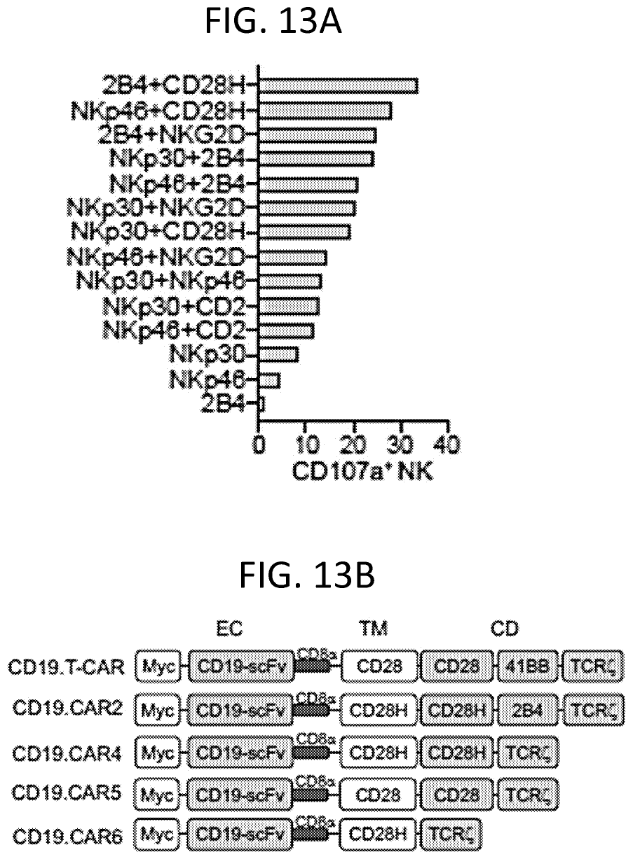

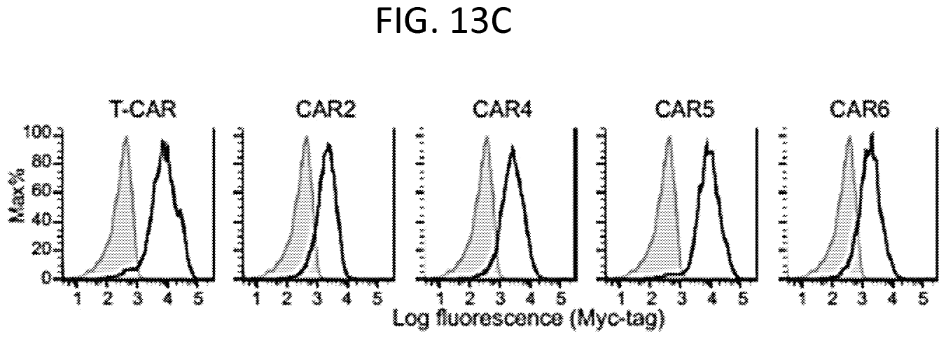

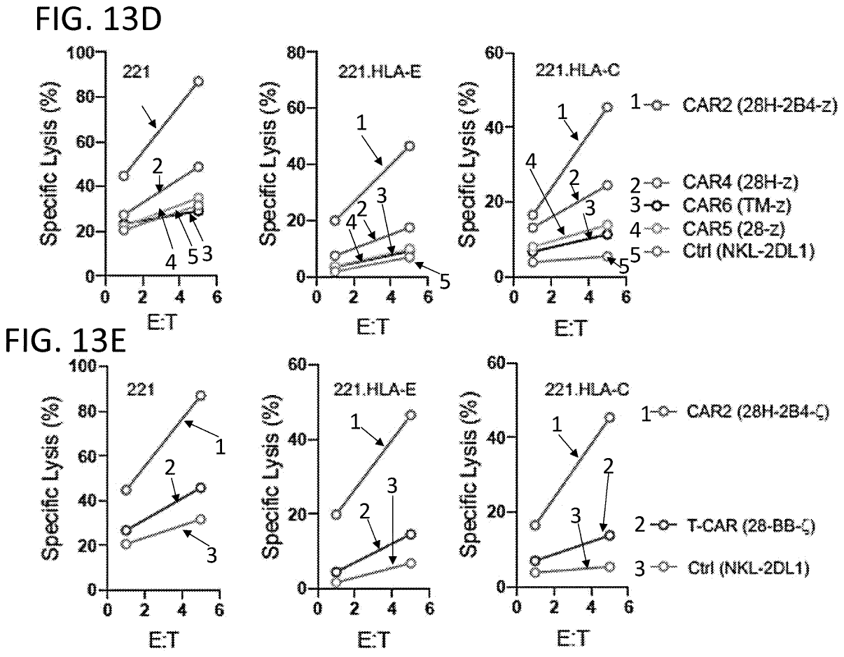

Provided herein are CARs that include an intracellular signaling domain from CD28H and at least one additional intracellular signaling domain (such as 1 or 2 additional intracellular signaling domains). In some embodiments, the CAR includes (in N- to C-terminal order) a target binding domain (such as an antigen binding domain), optionally a hinge (or “stem”) domain, a transmembrane domain, and an intracellular domain, including a first intracellular signaling domain from CD28H and a second intracellular signaling domain. The first and second intracellular signaling domains can be in any order. In particular embodiments, the intracellular domain includes a first intracellular signaling domain from CD28H and a second intracellular signaling domain from 2B4, TCRζ, FcER1γ, or DAP12. Exemplary CARs include, but are not limited to, those schematically illustrated in FIG. 13B.

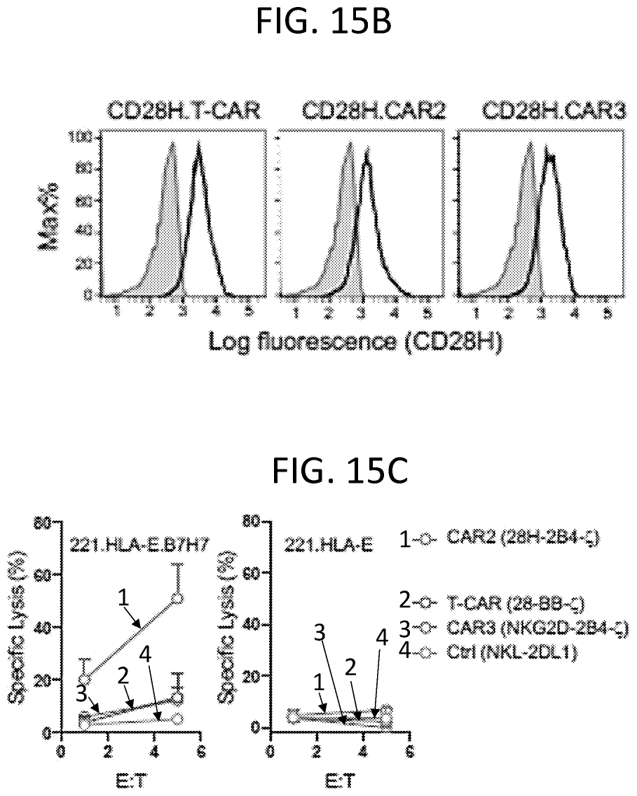

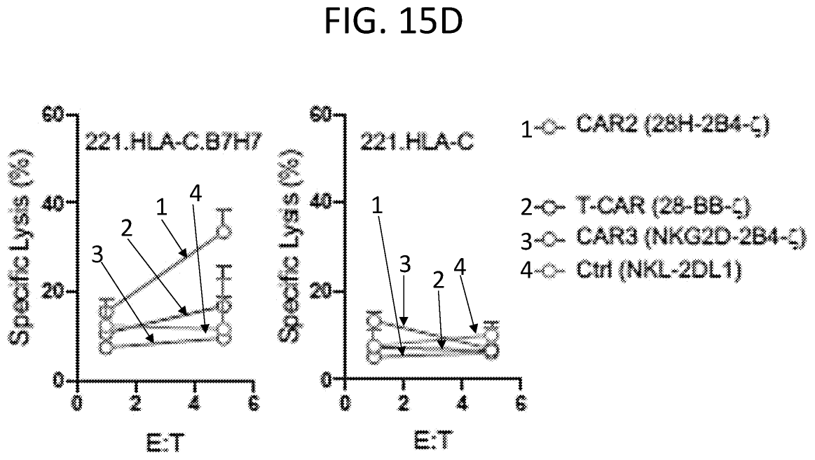

Also provided are CARs that include an intracellular signaling domain from CD28H and at least two additional intracellular signaling domains. In some embodiments, the CAR includes (in N- to C-terminal order) a target binding domain (such as an antigen binding domain), optionally a hinge (or “stem”) domain, a transmembrane domain, and an intracellular domain including a first intracellular signaling domain from CD28H, a second intracellular signaling domain from 2B4, and a third intracellular signaling domain. The first, second, and third intracellular signaling domains can be in any order. In some examples, the third intracellular signaling domain is from TCRζ, FcεR1γ, or DAP12. Exemplary CARs include, but are not limited to, those schematically illustrated in FIG. 15A and those described in Table 2.

In some embodiments, the first intracellular signaling domain is from CD28H and includes an amino acid sequence at least 90%, at least 95%, at least 96%, at least 97%, at least 98%, or at least 99% identical to, or including or consisting of amino acids 172-282 of SEQ ID NO: 2. In additional examples, the CD28H intracellular signaling domain is encoded by a nucleic acid molecule with at least 90%, at least 95%, at least 96%, at least 97%, at least 98% or at least 99% identical to, or including or consisting of nucleotides 514-846 of SEQ ID NO: 1. In other examples, the nucleic acid encoding the CD28H intracellular signaling domain is codon-optimized and includes a nucleic acid molecule with at least 90%, at least 95%, at least 96%, at least 97%, at least 98% or at least 99% identical to, or including or consisting of nucleotides 1018-1350 of SEQ ID NO: 15.

In some embodiments, the intracellular domain includes a second intracellular signaling domains. The second intracellular signaling domain may be C-terminal to the first intracellular signaling domain, or may be N-terminal to the first intracellular signaling domain. In one example, the second intracellular signaling domain is from TCRζ. In some examples, the second intracellular signaling domain includes an amino acid sequence at least 90%, at least 95%, at least 96%, at least 97%, at least 98%, or at least 99% identical to, or including or consisting amino acids 283-395 of SEQ ID NO: 2. In additional examples, the TCRζ intracellular signaling domain is encoded by a nucleic acid molecule with at least 90%, at least 95%, at least 96%, at least 97%, at least 98% or at least 99% identical to, or including or consisting of nucleotides 847-1185 of SEQ ID NO: 1. In other embodiments, the second intracellular signaling is from 2B4 and includes an amino acid sequence at least 90%, at least 95%, at least 96%, at least 97%, at least 98%, or at least 99% identical to, or including or consisting of amino acids 171-291 of SEQ ID NO: 10. In additional examples, the 2B4 intracellular signaling domain is encoded by a nucleic acid molecule with at least 90%, at least 95%, at least 96%, at least 97%, at least 98% or at least 99% identical to, or including or consisting of nucleotides 514-873 of SEQ ID NO: 9. In still further embodiments, the second intracellular signaling domain is from FcεR1⁻ or DAP12.

In some embodiments, the intracellular domain includes a second intracellular domain from 2B4 (for example, as described above) and a third intracellular signaling domain. In particular embodiments, the third intracellular signaling domain is from TCRζ, FcεR1γ, or DAP12, for example, as described above.

In some embodiments, the target binding domain (also referred to as an antigen binding domain) is an antigen binding domain or scFv that binds to a target of interest, such as a protein expressed on a cancer or tumor cell or a tumor associated antigen. Any target binding domain can be inserted in the CARs described herein. In some embodiments, the target binding domain binds to a protein expressed on a hematological malignancy or a solid tumor. In some non-limiting examples, the antigen binding domain binds to CD19 (such as an scFv that binds to CD19). In other examples, the target binding domain binds to B7H7 (such as the extracellular domain of CD28H). Exemplary targets of the extracellular binding domain and corresponding malignancies are shown in Table 1.

TABLE 1
Exemplary extracellular antigen binding domain targets and malignancies

| Target | Malignancies |
|---|---|
| CD19 | Acute lymphoblastic leukemia (ALL), |
| | lymphoma, Non-Hodgkin lymphoma |
| CD22 | ALL, lymphoma, Non-Hodgkin lymphoma |
| B cell maturation factor | Multiple myeloma |
| (BCMA) | |
| CD171 | Neuroblastoma |
| Epidermal growth factor | Glioblastoma |
| receptor variant III | |
| (EGFRvIII) | |
| Interleukin-13 receptor | Glioblastoma |
| alpha (IL13Ra) | |
| Mesothelin | Ovarian cancer, cervical cancer, breast cancer, |
| | Fallopian tube cancer, pancreatic cancer, lung |
| | cancer, colorectal cancer, peritoneal |
| | carcinoma |
| Mucin 16 (MUC-16) | Ovarian cancer |
| Mucin 1 (Muc1) | Sarcoma, breast cancer, cervical cancer, |
| | pancreatic cancer, lung cancer, liver cancer, |
| | glioma, colorectal cancer, gastric cancer |
| Receptor tyrosine | Breast cancer, ovarian cancer, lung |
| kinase like orphan | adenocarcinoma, lymphoblastic leukemia |
| receptor 1 (ROR-1) | |
| Prostate Stem Cell | Pancreatic cancer, lung cancer |
| Antigen (PSCA) | |
| CD33 | Myeloid leukemia |
| Prostate specific | Prostate cancer, bladder cancer, cervical |
| membrane antigen | cancer |
| (PSMA) | |
| CD123 | Acute myeloid leukemia (AML), leukemia |
| CD70 | B cell malignancies, breast cancer, ovarian |
| | cancer, pancreatic cancer, melanoma, renal |
| | cell cancer |
| Human epidermal growth | Breast cancer, ovarian cancer, lung cancer, |
| factor receptor 2 (HER2) | gastric cancer, colorectal cancer, pancreatic |
| | cancer, glioblastoma, glioma |
| Carcinoembryonic | Breast cancer, lung cancer, colorectal cancer, |
| antigen (CEA) | gastric cancer, pancreatic cancer, liver |
| | metastases |
| GTPase-activating | Solid tumors |
| protein (GAP) | |
| CD5 | T cell ALL, T cell non-Hodgkin lymphoma |
| CD38 | Multiple myeloma |
| Ephrin type-A receptor 2 | Glioma |
| (EphA2) | |
| Fibroblast activation | Mesothelioma |
| protein alpha (FAP) | |
| Ganglioside G2 (GD2) | Glioma, neuroblastoma, sarcoma, cervical |
| | cancer |
| Epithelial cell adhesion | Breast cancer, prostate cancer, colon cancer, |
| molecule (EpCam) | pancreatic cancer, gastric cancer, hepatic |
| | carcinoma, esophageal carcinoma, lymphoma, |
| | leukemia |
| CD133 | AML, breast cancer, ovarian cancer, colorectal |
| | cancer, glioma, pancreatic cancer, liver cancer |
| Glypican 3 (GPC3) | Lymphoma, leukemia, pancreatic cancer, |
| | colorectal cancer, lung cancer, liver cancer |
| B7H7 (HHLA2) | Breast cancer |

Thus, in some embodiments, the extracellular target binding domain binds to one or more of CD19, CD22, B cell maturation factor (BCMA), CD171, epidermal growth factor receptor variant III (EGFRvIII), interleukin-13 receptor alpha (IL-13Ra), mesothelin, mucin 16, mucin 1, receptor tyrosine kinase-like orphan receptor 1 (ROR-1), prostate stem cell antigen (PSCA), CD33, prostate-specific membrane antigen (PMSA), CD123, CD70, human epidermal growth factor receptor 2 (HER2), carcinoembryonic antigen (CEA), GTPase-activating protein (GAP), CD5, CD38, ephrin type-A receptor 2 (EphA2), fibroblast activation protein alpha (FAP), ganglioside G2 (GD2), epithelial cell adhesion molecule (EpCam), CD133, and glypican 3 (GPC3). This list is non-limiting, and additional extracellular targeting domains can also be utilized. In particular examples, the targeting domain is a scFv that binds to CD19 (such as amino acids 32-273 of SEQ ID NO: 12, or encoded by nucleotides 94-819 of SEQ ID NO: 11). In other particular examples, the extracellular target binding domain is a CD28H extracellular domain (such as amino acids 1-150 of SEQ ID NO: 8, or encoded by nucleotides 1-450 of SEQ ID NO: 7).

In some embodiments, the extracellular region of the CAR further includes a signal sequence domain, e.g., N-terminal to the antigen binding domain, for example, to facilitate expression of the CAR on the cell surface. In some examples, the signal sequence domain may be cleaved off of the CAR, during synthesis and/or upon expression on the cell surface. Therefore, in some embodiments, the CAR lacks a signal sequence domain. The signal sequence domain can include any suitable signal peptide sequence. In one non-limiting example, the signal sequence domain is a CD8α signal sequence. In some examples, the signal sequence includes or consists of amino acids 1-21 of SEQ ID NO: 16 or is encoded by nucleotides 1-63 of SEQ ID NO: 15. In another non-limiting example, the signal sequence domain is a human granulocyte-macrophage colony-stimulating factor (GM-CSF) signal sequence.

In other embodiments, the extracellular region of the CAR further includes a detectable label or tag, for example to facilitate detecting expression of the CAR in NK cells. In some examples, the detectable label or tag is N-terminal to the antigen binding domain; however, one of skill in the art could locate the detectable label or tag elsewhere in the construct, for example, C-terminal to the intracellular domains. In one non-limiting example, the detectable label or tag is a Myc tag. Other detectable labels or tags can be selected, including, but not limited to a fluorescent label.

In some embodiments, the disclosed CARs also include a hinge (or “stem”) domain, which is in some examples a spacer between the extracellular targeting domain and the transmembrane region. However, in other embodiments, the CAR does not include a hinge domain. In some embodiments, the extracellular hinge domain is a CD8α hinge domain. In some examples, the hinge domain is a CD8α hinge domain at least 90%, at least 95%, at least 96%, at least 97%, at least 98% or at least 99% identical to, or including or consisting of the amino acid sequence of amino acids 274-318 of SEQ ID NO: 12. In additional examples, the CD8α hinge domain is encoded by a nucleic acid molecule with at least 90%, at least 95%, at least 96%, at least 97%, at least 98% or at least 99% identical to, or including or consisting of nucleotides 820-954 of SEQ ID NO: 11. In other examples, the hinge domain is included in the CD28H sequences utilized in the CARs disclosed herein (for example, the hinge domain between the Ig domain and the transmembrane domain of CD28H).

The disclosed CARs include a transmembrane domain that is linked to the hinge domain, if present (e.g., C-terminal to the hinge domain) and the intracellular domain (e.g., N-terminal to the intracellular domain). In other examples, the transmembrane domain is linked to the extracellular targeting domain (e.g., C-terminal to the extracellular targeting domain) and the intracellular domain (e.g., N-terminal to the intracellular domain), if a hinge domain is not present. In some embodiments, the transmembrane domain is a CD28H transmembrane domain. In other examples, the transmembrane domain is a CD28 transmembrane domain, an NKp30 transmembrane domain, an NKp46 transmembrane domain, a CD16 (FCGR3A) transmembrane domain, an NKp44 transmembrane domain, or a KIR2DS4 transmembrane domain. In one specific example, the transmembrane domain is from CD28H. In some examples, the transmembrane domain is a CD28H transmembrane domain with at least 90%, at least 95%, at least 96%, at least 97%, at least 98% or at least 99% identical to, or including or consisting of amino acids 319-339 of SEQ ID NO: 16. In additional examples, the CD28H transmembrane domain is encoded by a nucleic acid molecule (which in at least some examples, may be codon-optimized) with at least 90%, at least 95%, at least 96%, at least 97%, at least 98% or at least 99% identical to, or including or consisting of nucleotides 955-1017 of SEQ ID NO: 15. In other specific examples, the transmembrane domain is at least 90%, at least 95%, at least 96%, at least 97%, at least 98% or at least 99% identical to, or including or consisting of amino acids 136-156 of SEQ ID NO: 23 (NKp30), amino acids 256-279 of SEQ ID NO: 22 (NKp46), amino acids 209-229 of SEQ ID NO: 21 (CD16), amino acids 193-213 of SEQ ID NO: 24 NKp44), or amino acids 246-265 of SEQ ID NO: 25 (KIR2DS4).

In some embodiments, the CAR includes an intracellular region C-terminal of the transmembrane domain and N-terminal to the first intracellular signaling domain (for example, an intracellular region of about 20-70 amino acids, such as about 25-50 amino acids, about 35-60 amino acids, or about 45-70 amino acids). In particular examples, this intracellular region is from NKp30, NKp46, CD16, NKp44, or KIR2DS4, and is linked C-terminal to their respective transmembrane domain (see e.g., Table 2 for examples). In particular examples, the intracellular region is at least 90%, at least 95%, at least 96%, at least 97%, at least 98% or at least 99% identical to, or includes or consists of amino acids 230-254 of SEQ ID NO: 21 (CD16), amino acids 280-304 of SEQ ID NO: 22 (NKp46), amino acids 157-201 of SEQ ID NO: 23 (NKp30), amino acids 214-276 of SEQ ID NO: 24 (NKp44), or amino acids 266-304 of SEQ ID NO: 25 (KIR2DS4).

Exemplary CARs of this disclosure include, but are not limited to, those schematically illustrated in FIGS. 13B and 15A and those described in Table 2.

In particular embodiments, the CAR includes an amino acid sequence with at least 90% sequence identity (for example, at least 91%, at least 92%, at least 93%, at least 94%, at least 95%, at least 96%, at least 97%, at least 98% at least 99% identity) to any one of SEQ ID NOs: 2, 6, 12, 16, and 20. In some examples, the CAR includes or consists of the amino acid sequence of any one of SEQ ID NOs: 2, 6, 12, 16, and 20. In particular examples, the signal peptide and/or Myc tag included in any one of SEQ ID NOs: 2, 6, 12, 16, and 20 are removed prior to use.

Also provided are nucleic acids encoding the CARs disclosed herein. In some embodiments, the CAR is encoded by a nucleic acid sequence with at least 90% sequence identity (for example, at least 91%, at least 92%, at least 93%, at least 94%, at least 95%, at least 96%, at least 97%, at least 98% or at least 99% sequence identity) to any one of SEQ ID NOs: 1, 5, 11, 15, and 19 or includes or consists of the nucleic acid sequence of any one of SEQ ID NOs: 1, 5, 11, 15, and 19. In particular examples, the signal sequence and/or Myc tag encoding sequences included in any one of SEQ ID NOs: 1, 5, 11, 15, and 19 are removed prior to use.

TABLE 2
Exemplary CARs

| | Extracellular | | | TM | Cytoplasmic | | |
|---|---|---|---|---|---|---|---|
| CD28H | Myc-tag | Anti-CD19 | CD8α | CD28H | CD28H | TCRζ | |
| plus one | | scFv | hinge | | | | |
| other | Myc-tag | Anti-CD19 | CD8α | CD28H | CD28H | FceR1γ | |
| signaling | | scFv | hinge | | | | |
| domain | Myc-tag | Anti-CD19 | CD8α | CD28H | CD28H | DAP12 | |
| | | scFv | hinge | | | | |
| | Myc-tag | Anti-CD19 | CD8α | CD28H | CD28H | 2B4 | |
| | | scFv | hinge | | | | |
| | Myc-tag | Anti-CD19 | CD8α | CD16^{a} | CD28H | 2B4 | |
| | | scFv | hinge | | | | |
| | Myc-tag | Anti-CD19 | CD8α | NKp46^{a} | CD28H | 2B4 | |
| | | scFv | hinge | | | | |
| | Myc-tag | Anti-CD19 | CD8α | NKp30^{a} | CD28H | 2B4 | |
| | Extracellular | scFv | hinge | TM | Cytoplasmic | | |
| | Myc-tag | Anti-CD19 | CD8α | NKp44^{a} | CD28H | 2B4 | |
| | | scFv | hinge | | | | |
| | Myc-tag | Anti-CD19 | CD8α | KIR2DS4^{a} | CD28H | 2B4 | |
| | | scFv | hinge | | | | |
| CD28H | Myc-tag | Anti-CD19 | CD8α | CD28H | CD28H | 2B4 | TCRζ |
| plus two | | scFv | hinge | | | | |
| other | Myc-tag | Anti-CD19 | CD8α | CD28H | CD28H | 2B4 | FceR1γ |
| signaling | | scFv | hinge | | | | |
| domains | Myc-tag | Anti-CD19 | CD8α | CD28H | CD28H | 2B4 | DAP12 |
| | | scFv | hinge | | | | |

^{a}These TM domains may optionally also include an intracellular region from the indicated protein, linked C-terminal to the TM domain

Also provided are functional variants of the CARs or the domains thereof described herein, which retain the biological activity of the CAR of which it is a variant or retains the biological activity of the particular domain. The functional variant can be at least about 80%, about 85%, about 90%, about 91%, about 92%, about 93%, about 94%, about 95%, about 96%), about 97%, about 98%, about 99% or more identical in amino acid sequence to the parent CAR or domain. Substitutions can be made, for example, in one or more of the extracellular targeting domain, hinge domain, transmembrane domain, and intracellular signaling domains.

In some examples, the functional variant includes the amino acid sequence of the parent CAR or domain with at least one conservative amino acid substitution (such as up to 10 conservative amino acid substitutions, for example, 1, 2, 3, 4, 5, 6, 7, 8, 9, or 10 conservative substitutions). In other examples, the functional variant includes the amino acid sequence of the parent CAR or domain with at least one non-conservative amino acid substitution (such as up to 10 non-conservative amino acid substitutions, for example, 1, 2, 3, 4, 5, 6, 7, 8, 9, or 10 non-conservative substitutions). In this case, the non-conservative amino acid substitution does not interfere with or inhibit the biological activity of the functional variant. The non-conservative amino acid substitution may enhance the biological activity of the functional variant, such that the biological activity of the functional variant is increased as compared to the parent CAR or domain.

The CARs or domains thereof can in some examples, include one or more synthetic amino acids in place of one or more naturally-occurring amino acids. Such synthetic amino acids include, for example, aminocyclohexane carboxylic acid, norleucine, a-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α,β-aminocycloheptane carboxylic acid, -(2-amino-2-norbomane)-carboxylic acid, γ-diaminobutyric acid, α,β-diaminopropionic acid, homophenylalanine, and α-tert-butylglycine. The CARs may be glycosylated, amidated, carboxylated, phosphorylated, esterified, N-acylated, cyclized via, e.g., a disulfide bridge, or converted into an acid addition salt and/or optionally dimerized or polymerized, or conjugated. In other embodiments, nucleic acids encoding the CARs may be codon-optimized, for example to improve expression in a cell of interest.

In some embodiments, a nucleic acid molecule encoding a disclosed CAR is included in an expression vector (such as a viral vector) for expression in a host cell, such as a T cell or NK cell. In some examples, the expression vector includes a promoter operably linked to the nucleic acid molecule encoding the CAR.

Additional expression control sequences, such as one or more enhancers, transcription and/or translation terminators, and initiation sequences can also be included in the expression vector. In some embodiments, a nucleic acid encoding a CAR provided herein is included in a viral vector. Examples of suitable virus vectors include retrovirus (e.g., a gammaretrovirus, such as MoMLV or lentivirus), adenovirus, adeno-associated virus, vaccinia virus, and fowlpox vectors. In some examples, the CAR encoding nucleic acid is included in a MoMLV vector, such as an SFG retroviral vector or a pHAGE-CPPT lentiviral vector. In another example, the CAR-encoding nucleic acid is included in a pCDH-EF1α-MSC-T2A-Puro lentivirus expression vector. In other examples, the vector may be a DNA vector. In further example, the CAR-encoding nucleic acid is included in a ⁻-retrovirus vector, such as MSGV-1.

In some examples, the vector further includes a nucleic acid sequence encoding at least one additional CAR. In some examples, the additional CAR is specific to an additional tumor antigen, for example, to increase specificity of targeting of the disclosed CAR to tumor cells expressing or overexpressing a target antigen.

### III. Cells Expressing Chimeric Adaptor Proteins

Also provided herein are cells (for example, immune cells) that express the disclosed CARs and compositions including cells expressing the disclosed CARs. In particular embodiments, the compositions include cells (such as NK cells or T cells) expressing a disclosed CAR and a pharmaceutically acceptable carrier.

In some embodiments, a nucleic acid molecule encoding a disclosed CAR is included in an expression vector (such as a viral vector) for expression in a host cell, such as a T cell or NK cell. In some examples, the expression vector includes a promoter operably linked to the nucleic acid molecule encoding the CAR. Additional expression control sequences, such as one or more enhancers, transcription and/or translation terminators, and initiation sequences can also be included in the expression vector.

The disclosed nucleic acids can be expressed in a host cell, such as a bacterial, plant, yeast, insect, or mammalian cell, for example, using an expression vector including a nucleic acid encoding the CAR. When the host is a eukaryote, methods of transfection of DNA such as calcium phosphate coprecipitation, microinjection, electroporation, insertion of a plasmid encased in liposomes, or virus vectors may be used. Eukaryotic cells can also be co-transformed with polynucleotide sequences encoding the CAR, and a second nucleic molecule encoding a selectable phenotype, such as the herpes simplex thymidine kinase gene. Another method is to use a eukaryotic viral vector, such as simian virus 40 (SV40), a lentivirus, or a retrovirus, to transduce or transform eukaryotic cells and express the CAR (see for example, Viral Expression Vectors, Springer Press, Muzyczka ed., 2011). In some examples, such expression systems are used to produce recombinant proteins in cells such as 293, COS, CHO, HeLa, or myeloma cell lines.

In some embodiments, a viral vector is utilized for expression of the CAR Viral vectors include, but are not limited to simian virus 40, adenoviruses, adeno-associated virus (AAV), lentiviral vectors, and retroviruses, such as gamma retroviruses. Retroviral vectors provide a highly efficient method for gene transfer into eukaryotic cells. Moreover, retroviral integration takes place in a controlled fashion and results in the stable integration of one or a few copies of the new genetic information per cell. Without being bound by theory, lentiviral vectors have the advantage over vectors derived from onco-retroviruses such as murine leukemia viruses in that they can transduce non-proliferating cells, such as hepatocytes. They also have the added advantage of low immunogenicity. In one non-limiting example, the vector is a lentivirus vector such as pELNS, for example, with an EF1a promoter. Other exemplary vectors include pLV-ER1a-IRES-Neo, with neomycin deleted or retroviral vector MSGV. In a specific example, the vector is a pCDH-EF1α-MCS-T2A Puro lentivirus expression vector, which can be used to produce lentivirus along with plasmids sPAX2 and pMD2.G, for example.

Also provided are immune cells (such as NK cells or T cells) expressing a CAR disclosed herein. The immune cells are transduced with an expression vector including a nucleic acid encoding a CAR. In some examples, the transduced cells are peripheral blood lymphocytes (for example, obtained from a subject), peripheral blood mononuclear cells (for example, obtained from a subject), isolated T cells (such as a primary T cell or T cells obtained from a subject), or isolated NK cells (such as a primary NK cell or NK cells obtained from a subject). T cells or NK cells can be obtained from a sample from a subject, for example, blood, plasma, bone marrow, lymph node, or thymus. In some examples, T cells or NK cells are also enriched, purified, and/or expanded from a sample from a subject, for example before and/or after transduction with the CAR expression vector.

### IV. Methods of Immunotherapy

Disclosed herein are methods of treating a subject with cancer (such as a hematological malignancy or a solid tumor) with a CAR. In some embodiments, the methods include administering to the subject a composition including a T cell or NK cell expressing a CAR disclosed herein (for example, transduced with a vector encoding the CAR) and a pharmaceutically acceptable carrier. In other examples, the methods include administering to the subject a pharmaceutical composition including an expression vector encoding a disclosed CAR and a pharmaceutically acceptable carrier. The extracellular targeting domain of the CAR is selected based on the cancer being treated, for example, as shown in Table 1.

Examples of hematological malignancies include leukemias, including acute leukemias (such as 11q23-positive acute leukemia, acute lymphocytic leukemia (ALL), T-cell ALL, acute myelocytic leukemia, acute myelogenous leukemia (AML), and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), lymphoblastic leukemia, polycythemia vera, lymphoma, diffuse large B cell lymphoma, Burkitt lymphoma, T cell lymphoma, follicular lymphoma, mantle cell lymphoma, Hodgkin disease, non-Hodgkin lymphoma, multiple myeloma, Waldenstrom macroglobulinemia, heavy chain disease, myelodysplastic syndrome, hairy cell leukemia, and myelodysplasia. In one non-limiting example, the hematological malignancy is acute lymphocytic leukemia (ALL).

Examples of solid tumors, include sarcomas (such as fibrosarcoma, myxosarcoma, liposarcoma, chondrosarcoma, osteogenic sarcoma, and other sarcomas), synovioma, mesothelioma, Ewing sarcoma, leiomyosarcoma, rhabdomyosarcoma, colon cancer, colorectal cancer, peritoneal cancer, esophageal cancer, pancreatic cancer, breast cancer (including basal breast carcinoma, ductal carcinoma and lobular breast carcinoma), lung cancer, ovarian cancer, prostate cancer, liver cancer (including hepatocellular carcinoma), gastric cancer, squamous cell carcinoma (including head and neck squamous cell carcinoma), basal cell carcinoma, adenocarcinoma, sweat gland carcinoma, medullary thyroid carcinoma, papillary thyroid carcinoma, pheochromocytoma, sebaceous gland carcinoma, papillary carcinoma, papillary adenocarcinoma, medullary carcinoma, bronchogenic carcinoma, hepatoma, bile duct carcinoma, choriocarcinoma, Wilms tumor, cervical cancer, fallopian tube cancer, testicular tumor, seminoma, bladder cancer (such as renal cell cancer), melanoma, and CNS tumors (such as a glioma, glioblastoma, astrocytoma, medulloblastoma, craniopharyrgioma, ependymoma, pinealoma, hemangioblastoma, acoustic neuroma, oligodendroglioma, meningioma, neuroblastoma and retinoblastoma). Solid tumors also include tumor metastases (for example, metastases to the lung, liver, brain, or bone).

A variety of pharmaceutically acceptable carriers can be used in the compositions provided herein, for example, buffered saline and the like, for introducing the cells or vectors to a subject. These solutions are sterile and generally free of undesirable matter. The compositions may be sterilized. In some examples, the compositions also include pharmaceutically acceptable auxiliary substances such as pH adjusting and buffering agents, toxicity adjusting agents, and preservatives, for example, sodium acetate, sodium chloride, potassium chloride, calcium chloride, sodium lactate and the like. The concentration in these formulations can vary, and will be selected primarily based on fluid volumes, viscosities, body weight and the like in accordance with the particular mode of administration selected and the subject's needs.

The precise amount of the composition to be administered can be determined by a physician with consideration of individual differences in age, weight, tumor size, extent of metastasis, and condition of the patient (subject). In some embodiments, a pharmaceutical composition comprising the T cells or NK cells expressing a CAR described herein is administered at a dosage of about 10⁴ to 10⁹ cells/kg body weight (for example, about 10⁴, 10⁵, 10⁶, 10⁷, 10⁸,or 10⁹ cells/kg), such as about 10⁴ to 10⁶ cells/kg, about 10⁵ to 10⁷ cells/kg, or about 10⁶ to 10⁸ cells/kg. Exemplary doses are about 10⁵ cells/kg to about 10⁹ cells/kg, such as about 10⁶ cells/kg, about 5×10⁶ cells/kg, about 10⁷ cells/kg, about 5×10⁷ cells/kg, about 10⁸ cells/kg, or about 5×10⁸ cells/kg. The population of modified T cells or NK cells is typically administered parenterally, for example intravenously; however, injection or infusion to a tumor or close to a tumor (local administration) or administration to the peritoneal cavity can also be used. One of skill in the art can determine appropriate doses and routes of administration.

In some examples, the composition (such as a composition including the T cells or NK cells expressing the CAR) is administered one or more times, such as 1, 2, 3, 4, 5, 6, 7, 8, 9, or 10 times. The composition can be administered by intravenous injection or infusion. In some examples, the composition is administered daily, weekly, bimonthly or monthly. If multiple doses are administered, the time between administrations may be 1, 2, 3, 4, 5, 6, 7, 8, 9, 10, 11, 12, 13, 14 days, or more. In some non-limiting examples, the composition is formulated for intravenous administration and is administered multiple times. The quantity and frequency of administration will be determined by such factors as the condition of the subject and the type and severity of the subject's disease, although appropriate regimens may be determined by clinical trials.

In some examples, the CAR-modified NK cells or T cells are able to persist and/or replicate in vivo in the subject, resulting in long-term persistence that can lead to sustained tumor control. In same examples, the NK or T cells administered to the subject, or the progeny of these cells, persist in the subject for at least two months, three months, four months, five months, six months, seven months, eight months, nine months, ten months, eleven months, twelve months, thirteen months, fourteen months, fifteen months, sixteen months, seventeen months, eighteen months, nineteen months, twenty months, twenty-one months, twenty-two months, twenty-three months, or for years after administration to the subject. In other embodiments, the cells or their progeny are present for less than six months, five months, four months, three months two months, or one month, e.g., three weeks, two weeks, one week, after administration of the cells to the subject.

In one embodiment, the CAR is introduced into cells, such NK cells or T cells, and the subject (such as a subject with cancer) receives an administration of the cells. In some embodiments, the methods include isolating NK or T cells from a subject, transforming the NK or T cells with an expression vector (such as a lentiviral vector or a retroviral vector) encoding the CAR, and administering the modified NK cells or T cells expressing the CAR to the subject for treatment. The NK cells or T cells can be autologous to a recipient or allogeneic (for example, the isolated and transformed NK cells or T cells are not from the subject being treated). In some examples, the subject may undergo an immunosuppressive regimen (e.g., lymphodepletion or partial lymphodepletion) prior to administering the modified NK cells or T cells. Immune system supportive therapies (such as IL-2 and/or G-CSF) may also be administered to the subject, for example to promote expansion of the modified cells in the subject and/or to support recovery of neutrophils.

In some embodiments, a population of cells including lymphocytes (such as PBMCs) can be obtained by any method, including, but not limited to apheresis. All or a portion of the population of cells can be utilized immediately or all or a portion of the cells can be cryopreserved for future use. When ready for use, all or a portion of the population of cells is thawed (if previously cryopreserved) and NK cells or T cells are activated, enriched, and/or expanded in culture. Methods of isolating, activating, and expanding NK cells or T cells are known in the art (e.g., WO 2018/006054 and WO 2018/022646, incorporated herein by reference in their entirety). The cells are transduced with a vector including a CAR. In particular examples, about 10⁷-10⁹ cells are transduced (for example, about 1×10⁷, 5×10⁷, 1×10⁸, 5×10⁸, or 1×10⁹ cells). Transduced T cells or NK cells can be expanded ex vivo and can be cryopreserved at appropriate dosage amounts (for example, about 10⁶ to 10¹² cells) following expansion. The transduced NK cells or T cells are thawed (if previously frozen), prior to administration to the subject. The subject may undergo an immunosuppressive regimen (e.g., lymphodepletion) prior to administering the modified NK cells or T cells. The modified NK cells or T cells are administered to the subject, for example by injection or infusion.

Treatment efficacy is monitored by methods such as tumor size, number of lesions, tumor stage, response rate, or other criteria. In some examples, a decrease in size of a primary tumor or metastases (for example, as defined by standard RECIST or irRECIST criteria) indicates inhibition of cancer in the subject. In other examples, progression-free survival and/or overall survival (for example, for 1 month, 3 months, 6 months, 9 months, 12 months, 18 months 2 years, or more, such as 1-12 months, 6-18 months, 1-2 years, or more) indicates inhibition of cancer in the subject. In other examples, one or more of persistence of circulating CAR-expressing NK cells or T cells, changes in immune cell subsets, and activation status of immune cells, as well as other immunologic determinants are evaluated, with clinical outcomes evaluated at baseline (e.g., prior to or at the time of administration of the modified cells), at different time points during treatment, and/or at the time of disease progression.

In some examples, the subject is also treated with one or more of surgery, chemotherapy, radiation, immunosuppressive agents, chemotherapeutic agents, or any combination of two or more thereof. Exemplary agents include, but are not limited to alkylating agents, such as nitrogen mustards (for example, chlorambucil, chlormethine, cyclophosphamide, ifosfamide, and melphalan), nitrosoureas (for example, carmustine, fotemustine, lomustine, and streptozocin), platinum compounds (for example, carboplatin, cisplatin, oxaliplatin, and BBR3464), busulfan, dacarbazine, mechlorethamine, procarbazine, temozolomide, thiotepa, and uramustine; antimetabolites, such as folic acid (for example, methotrexate, pemetrexed, and raltitrexed), purine (for example, cladribine, clofarabine, fiudarabine, mercaptopurine, and thioguanine), pyrimidine (for example, capecitabine), cytarabine, fluorouracil, and gemcitabine; plant alkaloids, such as podophyllum (for example, etoposide, and teniposide), taxane (for example, docetaxel and paclitaxel), vinca (for example, vinblastine, vincristine, vindesine, and vinorelbine); cytotoxic/antitumor antibiotics, such as anthracycline family members (for example, daunorubicin, doxorubicin, epirubicin, idarubicin, mitoxantrone, and valrubicin), bleomycin, hydroxyurea, and mitomycin; topoisomerase inhibitors, such as topotecan and irinotecan; monoclonal antibodies, such as alemtuzumab, bevacizumab, cetuximab, gemtuzumab, rituximab, panitumumab, atezolizumab, avelumab, ipilimumab, ofatumumab, nivolumab, pembrolizumab, rituximab, durvalumab, and trastuzumab; photosensitizers, such as aminolevulinic acid, methyl aminolevulinate, porfimer sodium, and verteporfin; proteasome inhibitors, such as bortezomib, carfilzomib, oprozomib, ixazomib, marizomib, and delanzomib; kinase inhibitors, such as gefitinib, imatinib, sunitinib, sorafenib, vemurafenib, trametinib, and ruxolitinib; growth factor receptor inhibitors, such as acitinib, erlotinib, cabozantinib, and crizotinib; mTOR inhibitors, such as everolimus, temsirolimus, and temisorotimus; and other agents, such as alitretinoin, altretamine, amsacrine, anagrelide, arsenic trioxide, asparaginase, bexarotene, celecoxib, denileukin diftitox, enzalutamide, flutamide, nilutamide, bicalutamide, topilutamide, apalutamide, estramustine, hydroxycarbamide, pentostatin, masoprocol, mitotane, pegaspargase, tamoxifen, clomifene, raloxifene, anastrozole, fulvestrant, and tretinoin. Additional agents include checkpoint inhibitors, such as antibodies (e.g., nivolumab, pembrolizumab, ipilimumab, durvalumab, and atezolizumab) or small molecule inhibitors (e.g., BMS-1001, BMS-1166, CCX4503). In some examples, a checkpoint inhibitor is not administered to the subject. One of ordinary skill in the art can select one or more available agents appropriate to the subject and condition being treated.

### EXAMPLES

The following examples are provided to illustrate certain particular features and/or embodiments. These examples should not be construed to limit the disclosure to the particular features or embodiments described.

### Example 1

### CD28 Homolog is a Strong Activator of Natural Killer Cells for Lysis of B7H7⁺ Tumor Cells

Data included in this example was published in Zhuang and Long, Cancer Immunol. Res. 7(6):939-951, published online on Apr. 24, 2019, which is incorporated by reference herein in its entirety.

### Materials and Methods

Plasmids: A plasmid containing B7H7 cDNA was obtained from Harvard PlasmID Database (#HsCD00044662). B7H7 cDNA was amplified and cloned into the EcoRI and NotI cloning sites of pAc5.1/V5-His A vector (Thermo Fisher Scientific) for expression in Drosophila S2 cells, and the EcoRI and 10 NotI cloning sites of pCDH-EF1-T2A-Puro vector (System Biosciences) for expression in human cell lines. The cDNA of CD28H was obtained from Harvard PlasmID Database (#HsCD00416184) in the vector pLX304. CD28H cDNA was amplified and cloned into the EcoRI and Nod cloning sites of pCDH-EF1-T2A-Puro lentivirus vector (System Biosciences) for transduction of human cell lines. CD28H mutants and chimeras were generated using the In-Fusion HD cloning kit (Clontech) and verified by sequencing. All of the cDNAs cloned into the PCDH vector were in frame with the 2A-peptide. Expressed proteins could be detected by anti-2A antibody in immunoblots. All plasmid constructions were carried out using the In-Fusion HD cloning kit (Clontech).

Cells: Human NK cells were isolated from peripheral blood of healthy U.S. donors by negative selection (STEMCELL Technologies). NK cells were resuspended in Iscove's modified Dulbecco's medium (IMDM; Gibco) supplemented with 10% human serum (Valley Biomedical) and used within 4 days. IL2 and PHA activated NK cells were cultured as described previously (Liu et al., Immunity 36:600-611, 2012). Briefly, freshly isolated NK cells were cultured with irradiated autologous feeder cells in OpTimizer (Invitrogen) supplemented with 10% purified IL2 (Hemagen), 100 units/ml recombinant IL2 (Roche) and 5 μg/ml phytohemagglutinin (PHA, Sigma), and expanded in the same medium without PHA and feeder cells. CD28H expression was tested after 2 weeks of activation. To obtain NK cells activated by NKp46 and CD2 plus IL2, freshly isolated NK cells were cultured in plates coated with 5 μg/ml CD2 and NKp46 mAbs, in the presence of 100 units/ml recombinant IL2 (Roche). CD28H expression was tested at day 3, day 5, and day 7. NKL cells (obtained from M. J. Robertson, Indiana University Cancer Research Institute, Indianapolis, IN) and KHYG-1 cells were cultured in IMDM Medium (Gibco) supplemented with 10% heat-inactivated fetal calf serum (Gibco), 2 mM L-Glutamine (Gibco), and 100 units/ml recombinant IL-2 (Roche). 721.221 cells (referred to as 221 cells), P815 cells (obtained from American Type Culture Collection, Manassas, VA), Daudi cells (ATCC Manassas, VA) and HDLM-2 cells (Ju et al., PNAS 113:1624-1629, 2016) (obtained from T. Waldmann, NCI, NIH) were cultured in RPMI 1640 medium (Gibco) containing 10% heat-inactivated fetal calf serum (Gibco) and 2 mM L-Glutamine (Gibco). 221 cells transfected with HLA-E (221.AEH), which included the HLA-A signal peptide to achieve proper HLA-E expression (Lee et al., PNAS 95:199-204, 1998), were a gift from D. Geraghty (Fred Hutchinson Cancer Research Center, Seattle). Lenti-X 293T cells (Clontech) were cultured in DMEM medium (Gibco) supplemented with 10% heat-inactivated fetal calf serum (Gibco) and 2 mM L-Glutamine (Gibco). Cells were mycoplasma-free, as tested by the NIH Office of Research Services. All cell lines used were maintained in culture for no longer than 2 months after thawing, and were authenticated by morphology, growth characteristics, expression of surface markers, and functional assays.

Transfection and lentivirus production: For S2 cells transfection, cells were transfected with plasmids for CD48 and B7H7 expression, both together or each one alone, together with a pAc5.1N5-His A-puro plasmid for selection in 6 μg/ml puromycin at 1/10^{th} the amount of the expression plasmids. Resistant cells were cloned, and tested for CD48 and B7H7 expression. For production of lentivirus, low-passage Lenti-X 293T cells (Clontech) were plated in a T75 flask 1 day before transfection. Cells were transfected with PEI Max (Polyethylenimine). Briefly, plasmids pMD2.G 1.2 μg, psPAX2 2.3 μg, PCDH 4.6 μg and 217 μl serum-free DMEM were mixed in a 15 ml Falcon tube. 65 μl of PEI Max 40K (Polysciences) stock solution (1 mg/ml) were added, and samples were vortexed briefly. After 10 min at room temperature, 8.6 ml DMEM media with 10% FCS were added to the tube. Culture medium for the 293T cells was replaced with the fresh medium containing the transfection reagent mixture. Two days after transfection, supernatants were collected, passed through a 0.45 μm filter, aliquoted and stored at −80° C. Supernatants for transduction were used either directly or after enrichment with PEG-it (System Biosciences) to increase virus titer. For transduction of human cell lines, lentivirus was added to cells together with polybrene to a final concentration of 8 μg/ml, and incubated for 2 days. Cells were centrifuged at 1200 rpm for 10 min, and resuspended in complete medium with pre-titrated concentrations of puromycin. Surface expression of transduced genes on puromycin-resistant cells was verified by flow cytometry.

Flow cytometry assays: Most flow cytometry assays were performed by incubating cells with premixed fluorophore-conjugated antibodies at 4° C. for 30 minutes. Staining for HLA-E was performed by first incubating cells with anti-HLA-E (3D12) or control mouse IgG1 (clone MOPC-21), followed by PE-conjugated polyclonal goat F(ab′)₂ anti-mouse IgG Fc (Jackson). NK cells expanded in IL2 were pre-incubated in 10% human serum for 30 minutes on ice to block Fc receptor CD16, prior to staining with antibodies. As resting NK cells were cultured in IMDM containing 10% human serum until use, no further Fc receptor blocking was needed. Cells were washed after staining, and analyzed on a LSR II (BD Biosciences) or LSRFortessa™ X-20 (BD Biosciences). Data were analyzed with FlowJo (FlowJo, LLC).

Degranulation and cytotoxicity assays: Redirected cytotoxicity assays were performed as described (Bryceson et al., Blood 107:159-166, 2006). Briefly, P815 cells were incubated with 5 μg/ml of the indicated combinations of mAbs to CD28H (R&D MAB83162), 2B4 (BioLegend 329502), NKp46 (BD 557847), NKG2D (R&D MAB139), CD2 (BD 555323), DNAM-1 (BD Biosciences 559787), CD16 (BD 555404) and CD56 (BD 555513) for 15 minutes at room temperature. Resting NK cells were added at an E:T ratio of 1:2, mixed and gently centrifuged at 300 rpm for 1 minute. After 2 hours at 37° C., cells were stained with Live/Dead-NIR (Thermo Fisher), anti-CD56-Bv421 (BD 562751) and anti-CD107a-PE (BD 555801) and analyzed by flow cytometry. Target cell lysis assays were either performed using the ToxiLight Non-Destructive Cytotoxicity BioAssay Kit (Lonza) following the manufacturer's instructions, or through a flow-based assay. Briefly, NK cells were incubated with PKH67-labeled target cells for 6 hours in IMDM medium with 10% FCS at the indicated E:T ratios. Cells were stained with Live/Dead NIR, and the lysis of target cells were determined by flow cytometry. For CD28H blocking, NK cells were pre-incubated with 10 μg/ml CD28H antibody (R&D Systems) for 15 min before mixing with 221.B7H7 cells. KHYG-1 and NKL cells were rested in complete IMDM medium without IL-2 for 1 day, prior to use in cytotoxicity assays.

S2 cell mixing and multiple functional assays: Resting NK cells were mixed with transfected S2 cells at an E:T ratio of 1:2. Cells were incubated in IMDM medium with 10% FCS for 2 hours, stained with Live/Dead-NIR, anti-CD56-Bv421 and anti-CD107a-PE, and analyzed by flow cytometry. To stain for intracellular cytokines and chemokines, NK cells and S2 cells were incubated for 1 hour, and in the presence of 3 μM GolgiStop (BD Biosciences) for 5 hours. For CD28H blocking, 10 μg/ml CD28H antibody (R&D Systems) was pre-incubated with NK cells for 15 min before mixing with S2 cells. Cells were stained with anti-CD107a-PE (BD 555801) and anti-CD56-Bv421 (BD 562751), fixed with 4% paraformaldehyde for 10 min, and permeabilized with the Intracellular Staining Permeabilization Wash Buffer (BioLegend). Cells were stained with anti-IFN-γ-APC (BioLegend 506510), anti-TNF-α-BV650 (BioLegend 502937), anti-MIP-1α-FITC (Invitrogen MA523564) and anti-MIP-1β-PerCP-Cy5.5 (BD 560688). Data were obtained on a LSR II (BD Biosciences) or LSRFortessa™ X-20 (BD Biosciences), and analyzed with FlowJo (FlowJo, LLC).

ADCC assays: S2 cells were pre-incubated with a rabbit anti-S2 serum diluted 1:10,000 for 15 min at room temperature. Resting NK cells were added at an E:T ratio of 1:2, mixed, and centrifuged at 300 rpm for 1 min. After 2 hours at 37° C. in IMDM medium with 10% FCS, cells were stained with Live/Dead-NIR, anti-CD56-Bv421, and anti-CD107a-PE, and analyzed by flow cytometry. For ADCC assays using Rituximab, 221 cells and Daudi cells were pre-incubated with 10 μg/ml Rituximab at room temperature for 15 min. Resting NK cells were added at an E:T ratio of 5:1, and incubated for 5 hours. Lysis of target cells was determined using the ToxiLight Non-Destructive Cytotoxicity BioAssay Kit (Lonza) following the manufacturer's instructions.

Statistical analysis: Statistical analysis was performed with GraphPad PRISM V7. Data are presented as mean±SEM, and compared by two-tailed Mann-Whitney test or Wilcoxon signed-rank test.

Human donors: Peripheral blood samples from healthy U.S. adults were obtained from the NIH Department of Transfusion Medicine in accordance with the Belmont Report, under an NIH Institutional Review Board-approved protocol (99-CC-0168) with informed written consent.

### Results

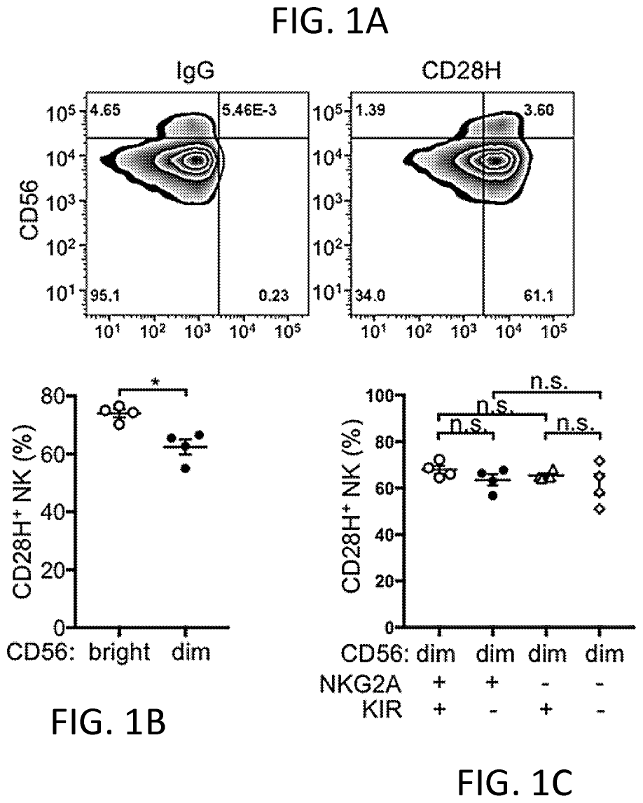

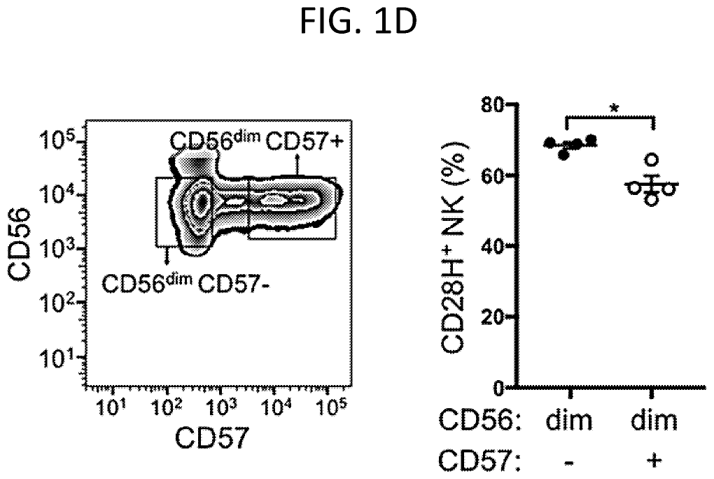

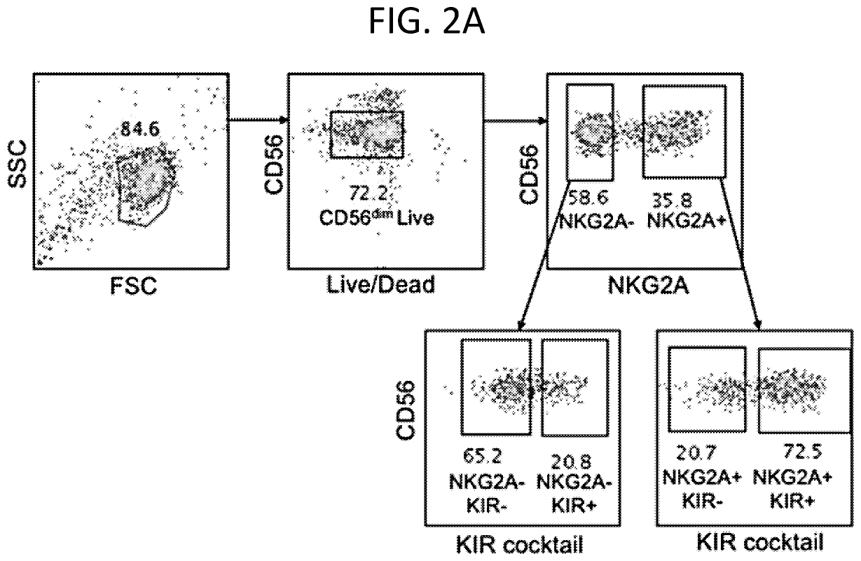

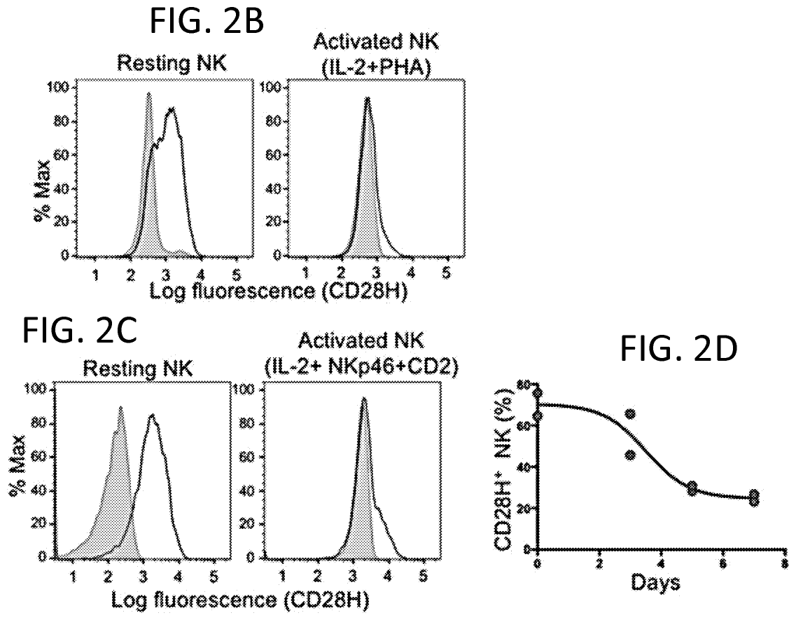

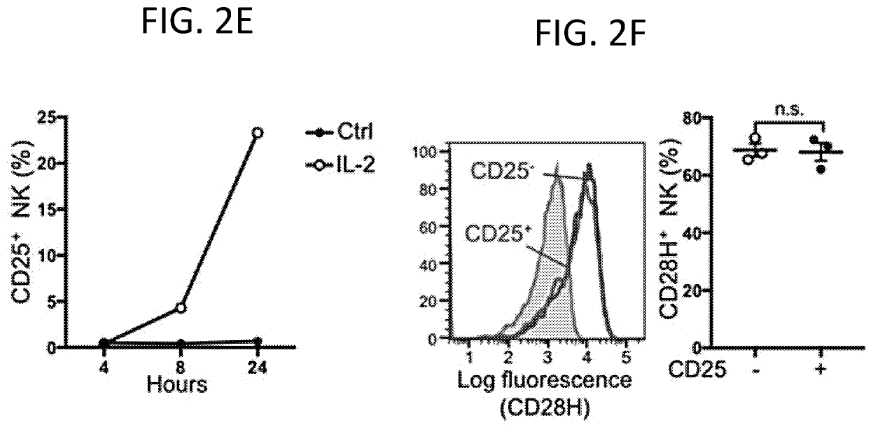

CD28H is expressed on primary, resting human NK cells: The majority of freshly isolated, human NK cells expressed CD28H at the cell surface (FIG. 1A). Human NK cells can be divided into CD56^{bright} and CD56^{dim} NK subsets based on the expression of CD56. The two subsets have distinct phenotypes and properties (Cooper et al., Trends Immunol. 22:633-640, 2001). We examined CD28H expression in the two NK subsets, and a greater fraction of the CD56^{bright} subset expressed CD28H (FIG. 1B). Most of the CD56^{bright} NK cells are phenotyped as CD56^{bright}CD16⁻KIR⁻NKG2A⁺CD57⁻, and represent a less mature NK cell population (Cooper et al., Trends Immunol. 22:633-640, 2001). Expression of KIR and NKG2A can divide CD56^{dim} NK cells into several subsets (FIG. 2A). However, no significant difference in CD28H expression was found between CD56^{dim} NK cells that were NKG2A⁺KIR⁺, NKG2A⁺KIR⁻, NKG2A⁻KIR⁺, and NKG2A⁻KIR⁻ NK (FIG. 1C). CD57 expression can also be used to further separate CD56^{dim} NK cells (Lopez-Verges et al., Blood 116:3865-3874, 2010). CD57⁺ NK cells are mature, terminal differentiated, and have decreased capacity for proliferation (Lopez-Verges et al. Blood 116:3865-3874, 2010). A lower proportion of CD57⁺ cells expressed CD28H, as compared to the CD56^{dim}CD57⁻ NK subset (FIG. 1D). Ex vivo expanded and activated NK cells are highly cytotoxic and have been used in clinical and basic research for decades (Bachanova et al., Crit Rev Oncog. 19:133-141, 2014; Granzin et al., Front Immunol. 8:458, 2017). A well-established strategy for NK cell expansion in culture is the combination of IL2 and PHA in the presence of irradiated autologous PBMC as feeder cells (Liu et al., Immunity 36:600-611, 20012; Granzin et al., Front Immunol. 8:458, 2017). CD28H expression on NK cells was lost after 2 weeks in IL2 and PHA (FIG. 2B), a result consistent with a quantitative proteomics analysis of immune cells, in which NK cells had been stimulated with IL2 and plate-coated mAbs to CD2 and NKp46 (Rieckmann et al., Nat Immunol. 18:583-593, 2017). Using the same stimulating conditions, we confirmed the decrease of CD28H expression by immunostaining (FIG. 2C). A reduction in the proportion of NK cells that expressed CD28H was observed 3 days after activation, and gradually dropped to ~20% of NK cells after 7 days of activation (FIG. 2D). We also tested expression of CD28H on NK cells activated for a shorter time in IL2. CD25 expression was used as a marker for activation (FIG. 2E). No significant difference was observed in CD28H expression on CD25⁺ and CD25⁻ NK cells after activation in IL2 for 24 hours (FIG. 2F). The results showed that expression of CD28H decreased only after long-term stimulation and expansion, but not by short-term activation with IL2.

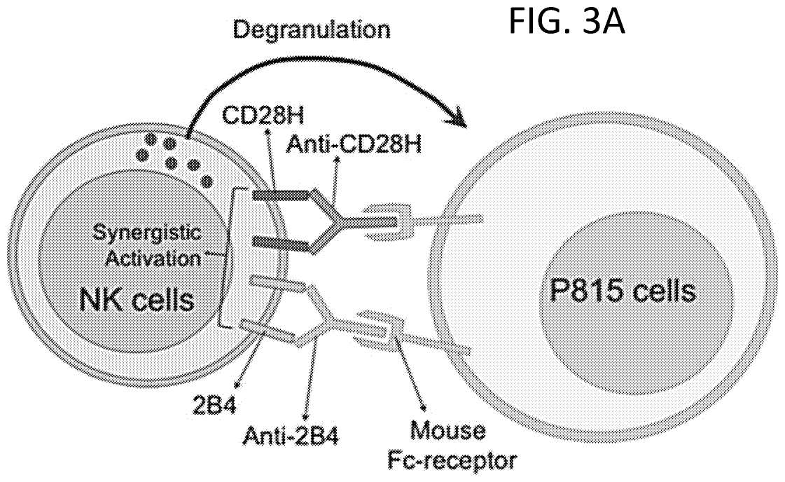

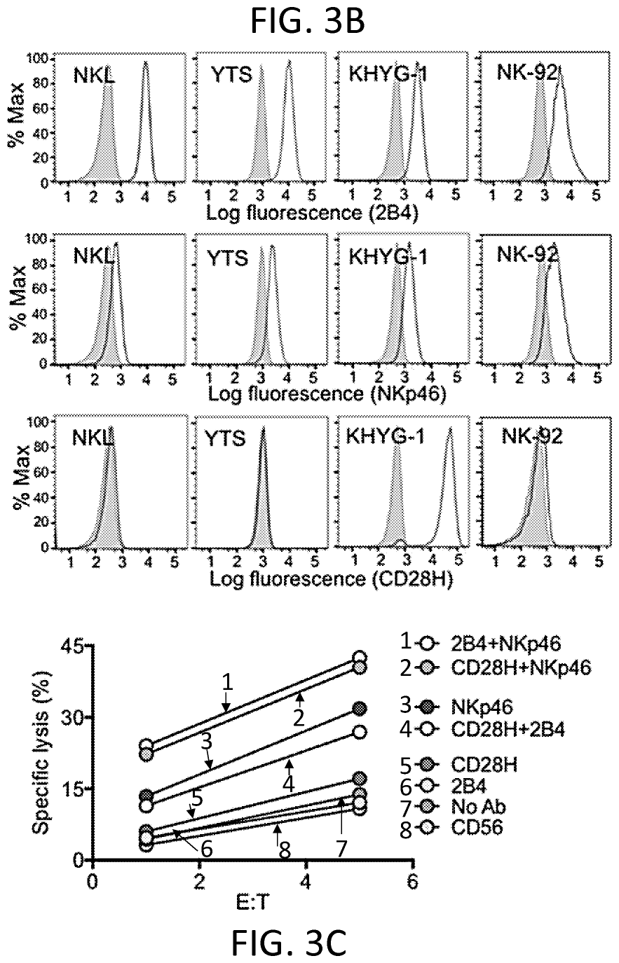

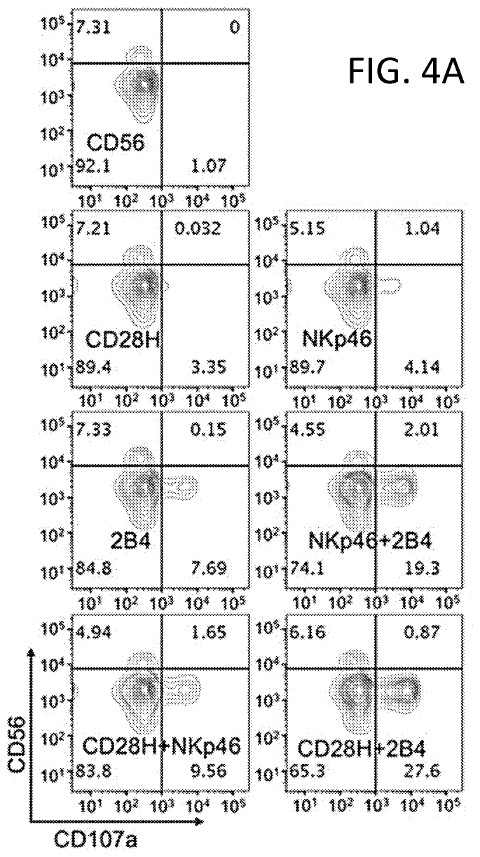

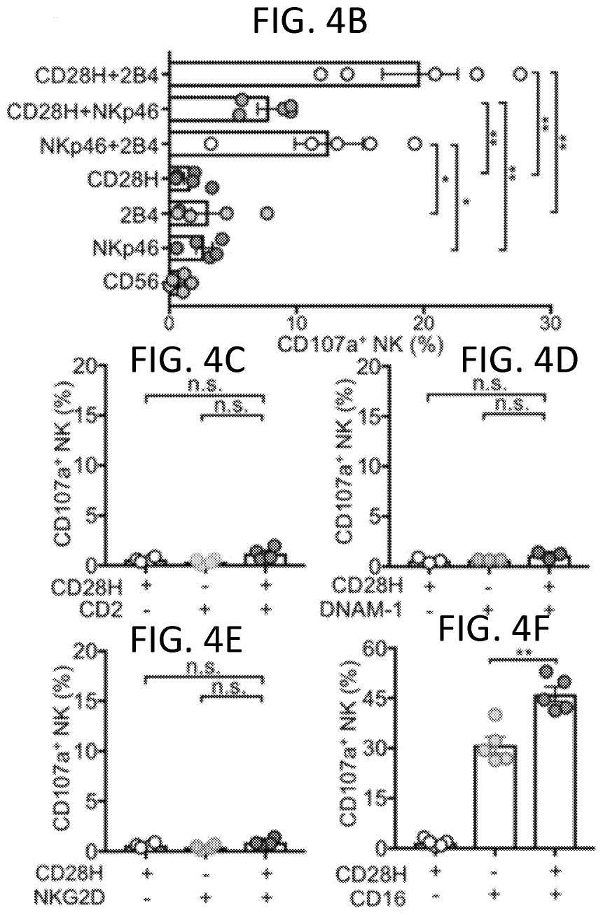

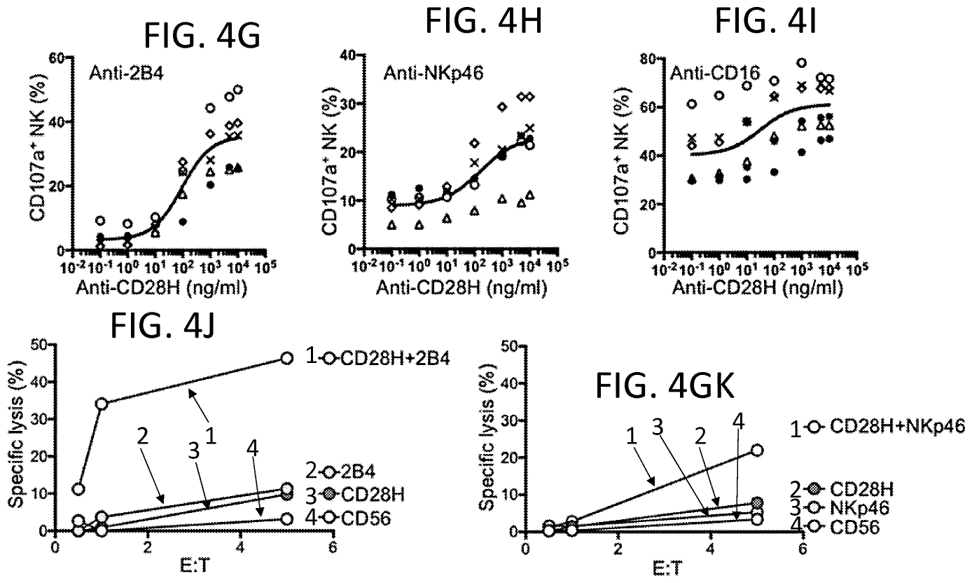

CD28H synergizes with 2B4 and NKp46 and enhances NK cell activation by CD16: NK-cell degranulation induced by CD28H alone and CD28H co-engaged with other receptors was tested using the redirected cytotoxicity assay (also known as reverse ADCC) (Bryceson et al., Blood 107:159-166, 2006; Vitale et al., J. Exp. Med. 187:2065-2072, 1998). The FcR⁺ mouse cell line P815 was mixed with monoclonal antibodies (mAbs) to CD28H and other receptors and incubated with NK cells. The F(ab′)₂ portions of the mAbs bound to their respective NK cell receptors, whereas the Fc fragments bound to FcR on mouse P815 cells. Activation of NK cells and lysis of P815 cells were induced by co-engagement of NK activation receptors (FIG. 3A). Strong NK-cell degranulation, determined by staining for CD56 and CD107a, occurred only after co-engagement of CD28H with either 2B4 or NKp46 (FIGS. 4A and 4B), but not with CD2, NKG2D, or DNAM-1 (FIGS. 4C-4E). CD56^{dim} NK cells in peripheral blood are competent in both cytotoxicity and cytokine production, whereas CD56^{bright} NK cells are less cytotoxic and have been viewed as cytokine producers (Cooper et al., Trends Immunol. 22:633-640, 2001; Fauriat et al., Blood 115:2167-2176, 2010). Therefore, although a slightly greater fraction of CD56^{bright} NK cells expressed CD28H (FIG. 1B), NK-cell degranulation was observed only on CD56^{dim} NK cells (FIG. 4A), consistent with previous studies (Bryceson et al., Blood 107:159-166, 2006; Bryceson et al., Blood 114:2657-2666, 2009). CD28H also enhanced the NK-cell degranulation induced by CD16 (FIG. 4F). To titrate the response of NK cells to stimulation through CD28H, mAbs to 2B4, NKp46, and CD16 were used at a constant concentration in redirected cytotoxicity assays in the presence of increasing concentrations of CD28H antibody (FIGS. 4G-4I). Enhancement of degranulation was observed with as low as 100 ng/ml CD28H antibody, and stimulation reached a plateau at about 5 μg/ml (FIGS. 4G-4I). Lysis of P815 cells in this redirected cytotoxicity assay was also investigated at different effector to target ratios (FIGS. 4J-4K). NK cells lysed P815 target cells efficiently upon co-engagement of CD28H with 2B4 and NKp46, and none of the three receptors alone triggered NK cell cytotoxicity (FIGS. 4J-4K).

Among four commonly used NK cell lines, namely NKL, YTS, KHYG-1, and NK-92, only KHYG-1 expressed CD28H, whereas all four expressed 2B4 and NKp46 (FIG. 3B). KHYG-1 cells lysed P815 target cells in the presence of antibodies to CD28H and 2B4, or CD28H and NKp46 (FIG. 3C). Therefore, the synergy of CD28H with 2B4 and NKp46 observed with resting NK cells was reproduced with KHYG-1 cells.

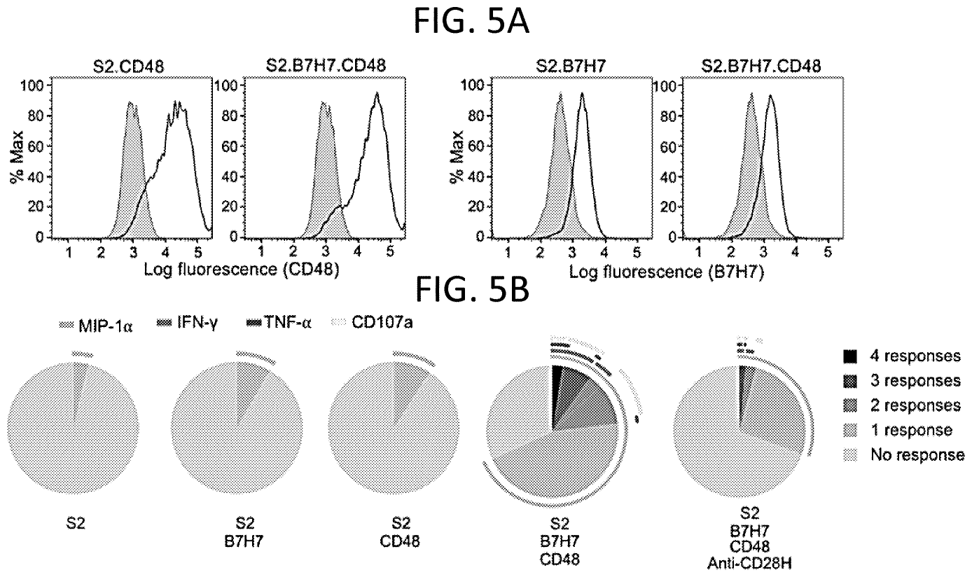

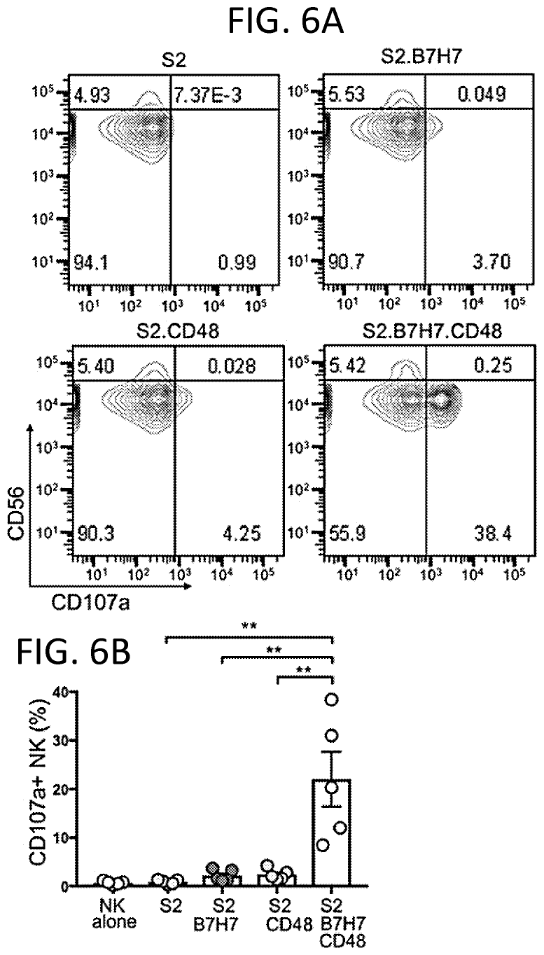

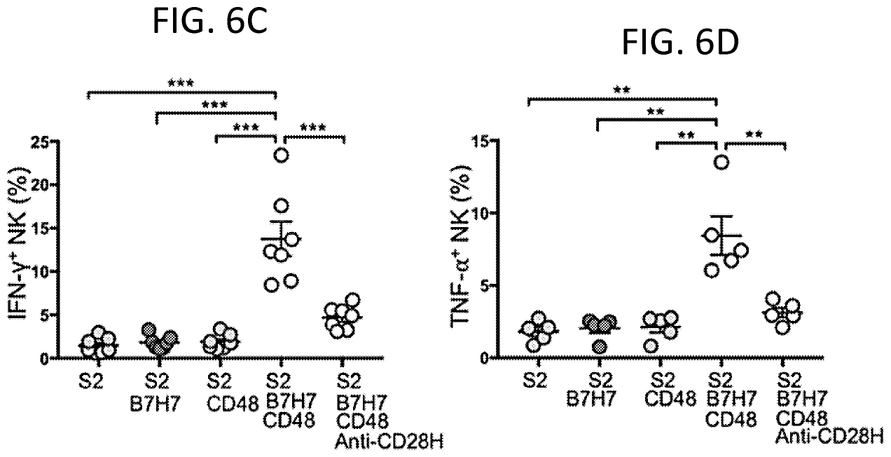

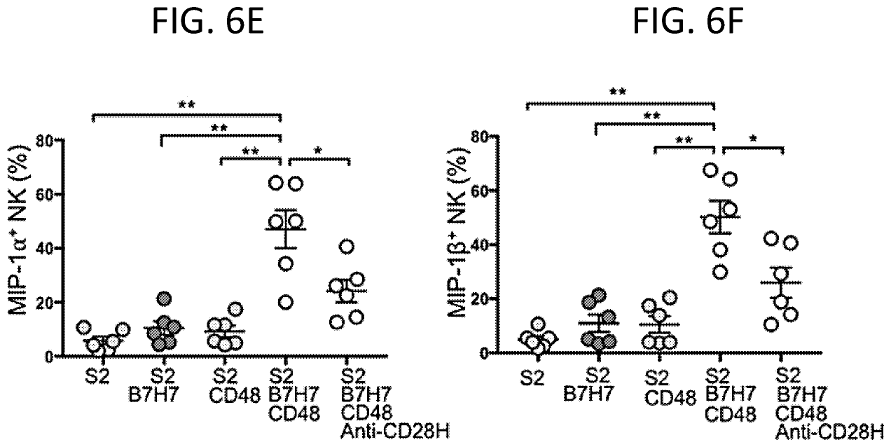

B7H7 and CD48 coexpression triggers NK-cell degranulation and cytokine production: To study the synergistic activation of NK cells by CD28H and 2B4 in the context of receptor-ligand interactions, we expressed B7H7 and CD48, either alone or in combination, in Drosophila S2 cells (FIG. 5A). CD48, which is expressed on most hematopoietic cells, is the ligand of 2B4. NK cells incubated with S2 cells expressing B7H7 (S2.B7H7) or CD48 (S2.CD48) for 2 hours did not degranulate (FIG. 6A). In contrast, S2 cells expressing both B7H7 and CD48 induced NK-cell degranulation (FIGS. 6A-6B). The results showed that engagement of both CD28H and 2B4 by their respective ligands lead to synergistic activation of NK cells. Coexpression of B7H7 and CD48 on S2 cells induced production of IFNγ, TNFα, MIP-1α, and MIP-1β in primary NK cells (FIGS. 6C-6F). As observed with NK-cell degranulation, cytokine and chemokine production was triggered only by synergy of CD28H and 2B4, but not by either receptor alone. Moreover, expression of IFNγ, TNF-α, MIP-1α, and MIP-1β was reduced in the presence of a CD28H blocking antibody (FIGS. 6C-6F), confirming stimulation through CD28H. Although CD48 and B7H7 alone stimulated a small proportion of NK cells to express MIP-1α, B7H7 and CD48 together induced strong NK cell responses—approximately 70% of NK cells expressed MIP-1α, and approximately 10% of NK cells had at least 3 different responses (FIG. 5B). It is worth noting that degranulating NK cells and IFNγ-producing NK cells were only partially overlapping. Moreover, the blocking antibody toward CD28H attenuated all of the NK responses.

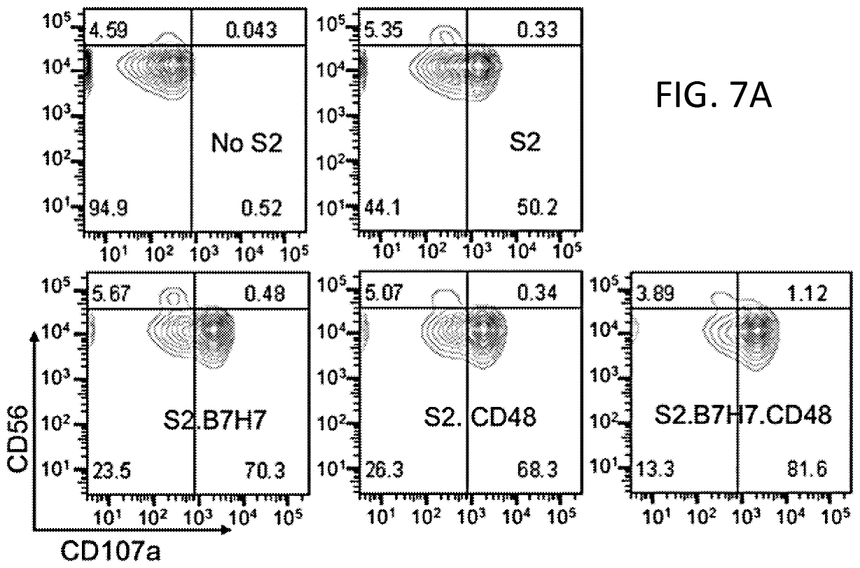

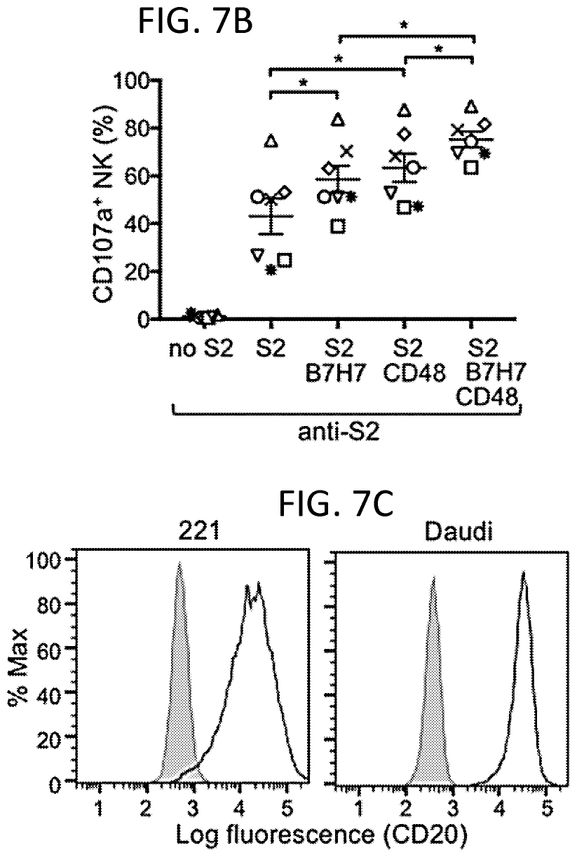

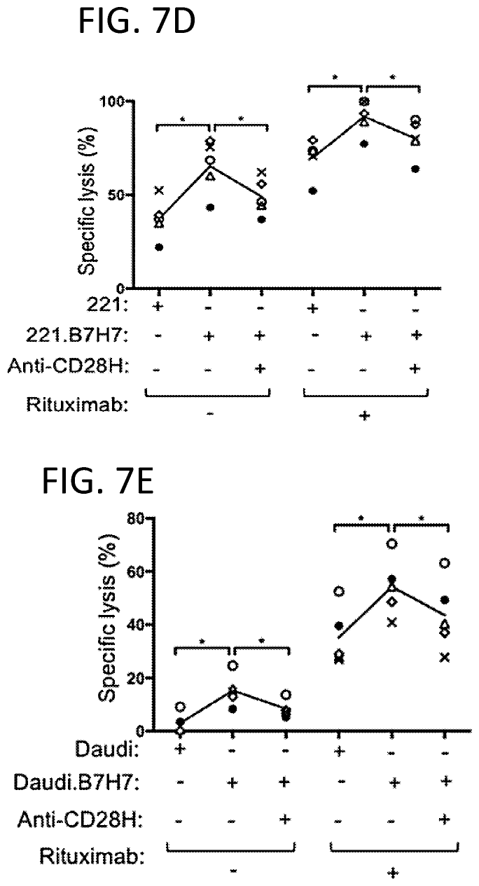

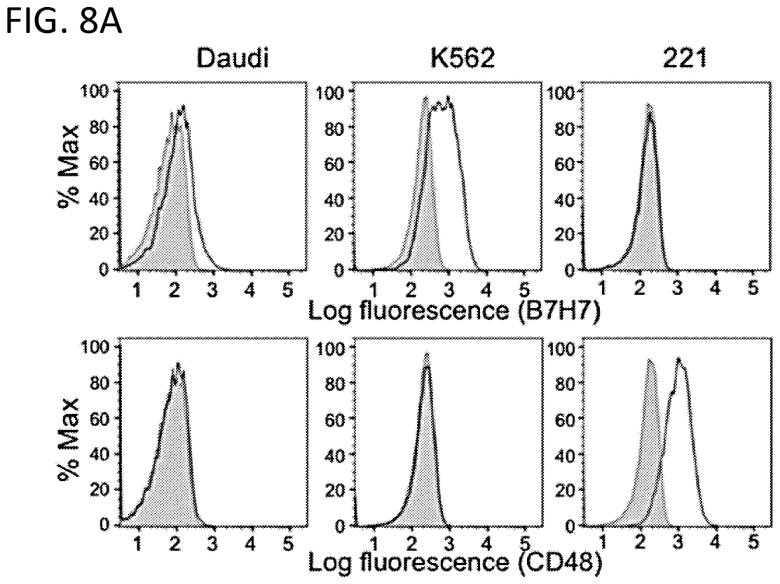

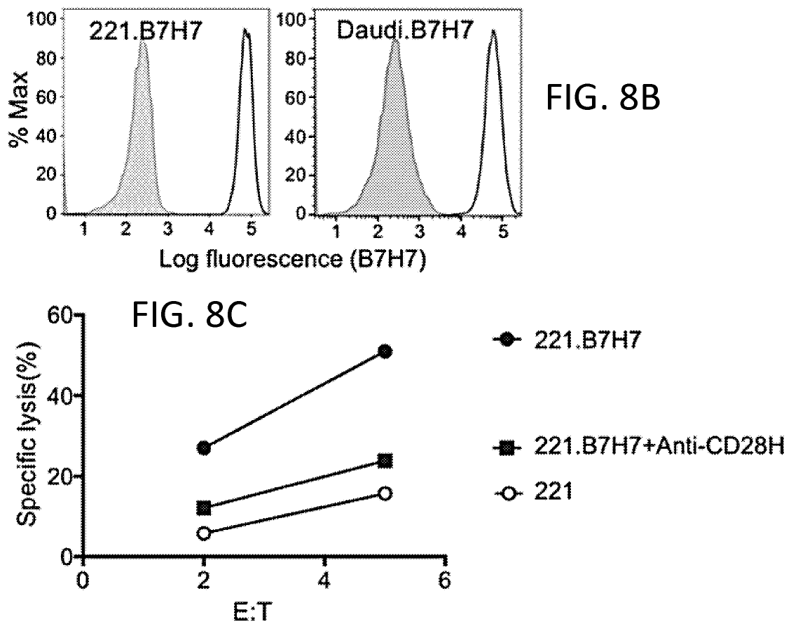

CD28H enhances CD16-mediated ADCC: To explore the possible enhancement of NK cell-mediated ADCC by CD28H, assays were performed with a rabbit antiserum to S2 cells (Bryceson et al., Blood 107:159-166, 2006). S2 cells were precoated with anti-S2 serum, followed by incubation with primary human NK cells. Expression of B7H7 in S2 cells enhanced NK activation in the ADCC assay, whereas expressing both B7H7 and CD48 increased NK degranulation even further (FIGS. 7A and 7B). NK cell-mediated ADCC plays a central role in the mechanism of action of therapeutic antibodies, such as rituximab (anti-CD20) and cetuximab (anti-EGFR) (Scott et al., Nat Rev Cancer 12:278-287, 2012). Three MHC class I-deficient cell lines commonly used as targets for NK cells were examined for expression of B7H7 (FIG. 8A). B7H7 was expressed by K562 cells, but not Daudi or 221 cells (FIG. 8A). Among these three cell lines, only 221 expressed CD48 (FIG. 8A). We transduced 221 and Daudi cells with a lentivirus for expression of B7H7 and obtained bright and uniform expression of B7H7 (FIG. 8B). Freshly isolated human NK cells lysed 221 cells expressing B7H7 (221.B7H7) more efficiently than untransfected 221 cells (FIG. 8C). The enhanced lysis of 221.B7H7 was blocked by a CD28H specific mAb (FIG. 8C). As Daudi and 221 cells are both CD20⁺ lymphoblastoid B-cell lines (FIG. 7C), we used rituximab to induce ADCC. Specific lysis of target cells in rituximab-induced ADCC was increased by the expression of B7H7, and the enhancement in killing was specifically blocked by CD28H antibody (FIGS. 7D and 7E). We concluded that the CD28H-B7H7 interaction enhanced natural killing of 221 and Daudi cells by primary NK cells, as well as lysis of 221 and Daudi cells through ADCC.

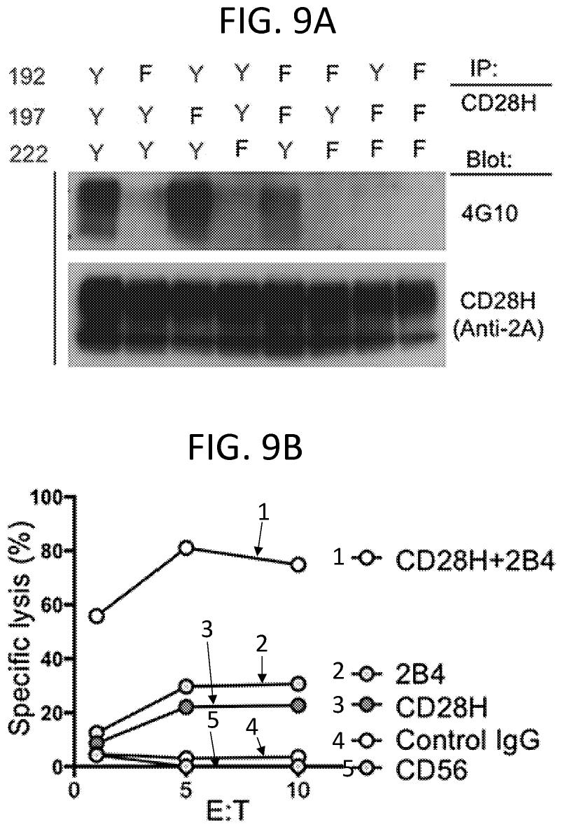

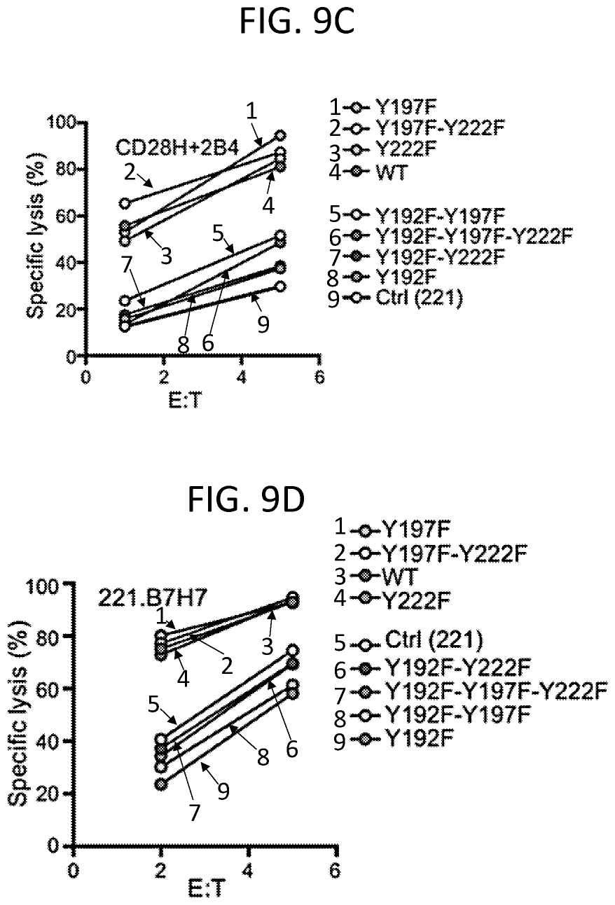

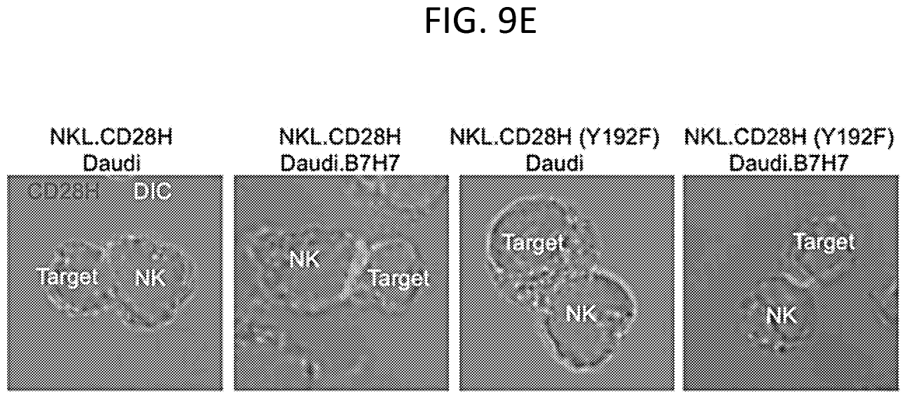

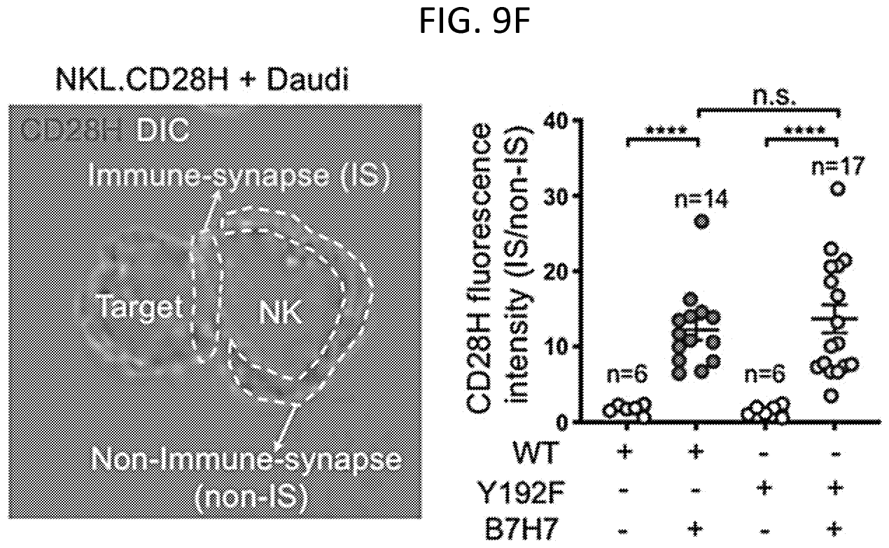

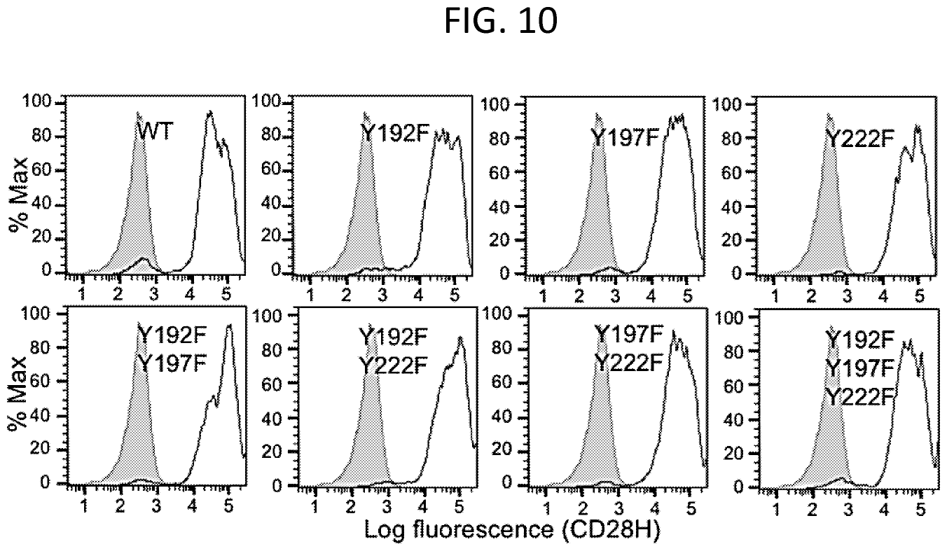

Tyr192 is essential for CD28H-mediated NK-cell activation: The CD28H cytoplasmic tail includes three tyrosines, which could potentially contribute to transduction of activation signals. Each tyrosine was replaced with a phenylalanine, in all possible combinations. CD28H wild-type and each one of the mutants were immunoprecipitated from transfected HEK293T cells after treatment with the tyrosine phosphatase inhibitor pervanadate. Tyrosine phosphorylation was greatly diminished by mutation of either Y192 or Y222 (FIG. 9A). The function of each of the mutants was tested by expressing them in an NK cell line. Exogenous expression of wild-type CD28H in the CD28H⁻ 2B4⁺ cell line NKL reproduced the synergy of CD28H and 2B4, as shown in the redirected ADCC assay with P815 cells (FIG. 9B). CD28H mutants were expressed in NKL cells to test the contribution of each tyrosine to CD28H-dependent activation of NK cells (FIG. 10). Single-site mutation of Y192 (Y192F) abolished synergistic activation by CD28H and 2B4 (FIG. 9C). Lysis of 221.B7H7 cells by NKL cells expressing wild-type CD28H and CD28H mutants was also investigated. Consistent with the redirected cytotoxicity assays, enhancement of killing of 221 cells by B7H7 expression was also lost with mutation Y192F (FIG. 9D), indicating that phosphorylation of Y192 may be essential for NK-cell activation. The Y192F mutation did not alter accumulation of CD28H at the immunological synapse (FIGS. 9E and 9F), suggesting that the accumulation of CD28H and its ability to signal for cytotoxicity were uncoupled. There are multiple stages in the formation of NK cell immunological synapses (Orange Nat. Rev. Immunol. 8:713-725, 2008). The accumulation of mutant Y192F at the synapse indicated that signals specifically triggered by Y192 are not required for that stage of synapse formation. In addition to the three tyrosine residues in the cytoplasmic tail, CD28H also contains a proline-rich domain, which may be involved in signal transduction.

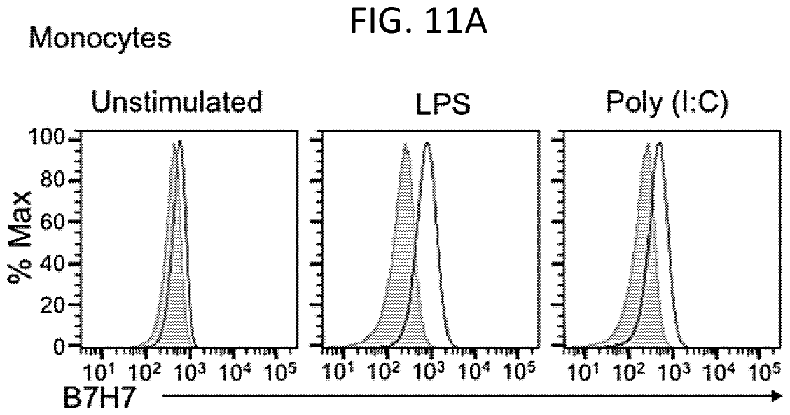

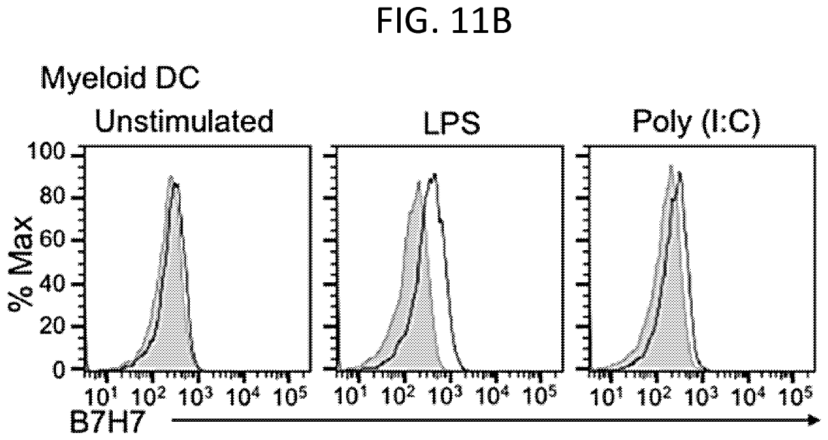

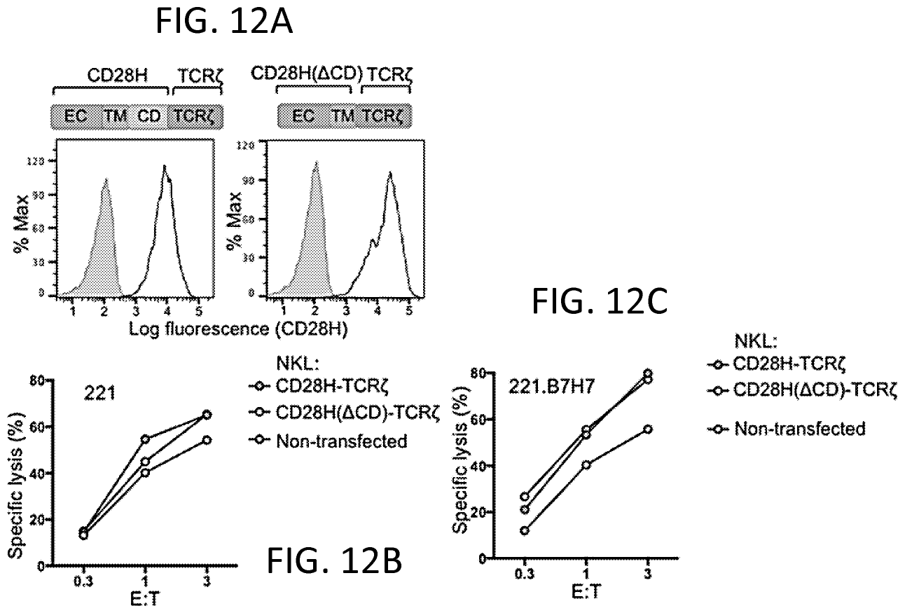

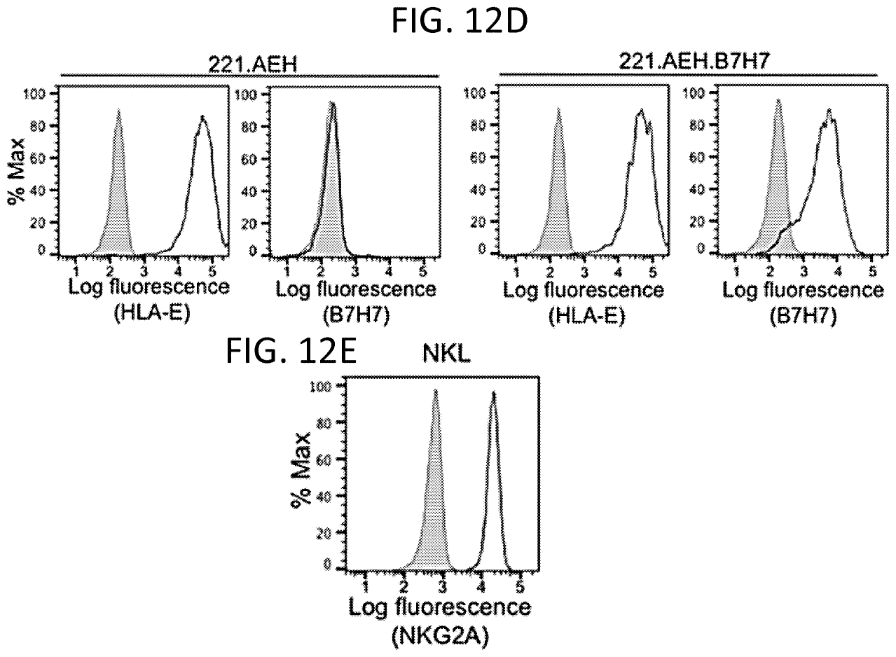

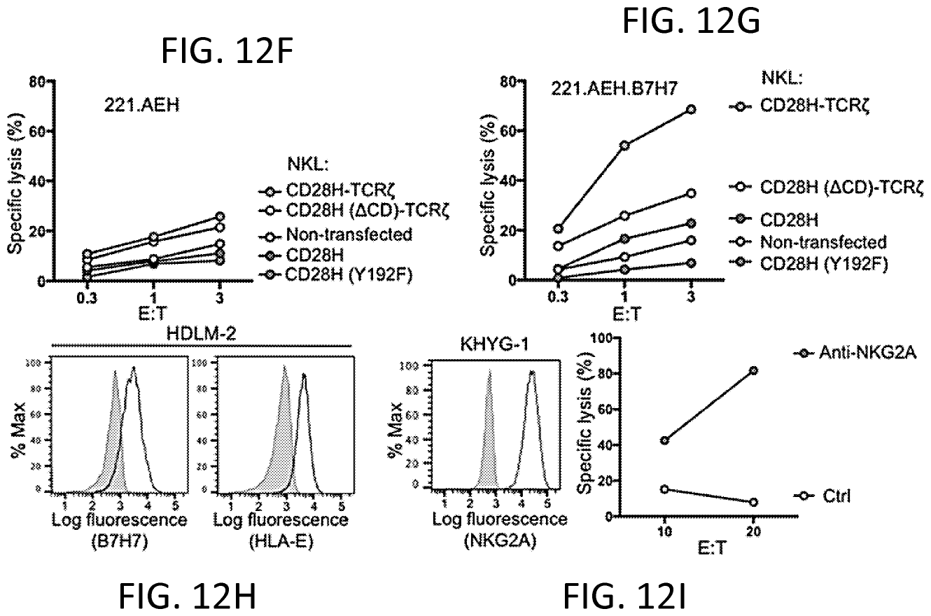

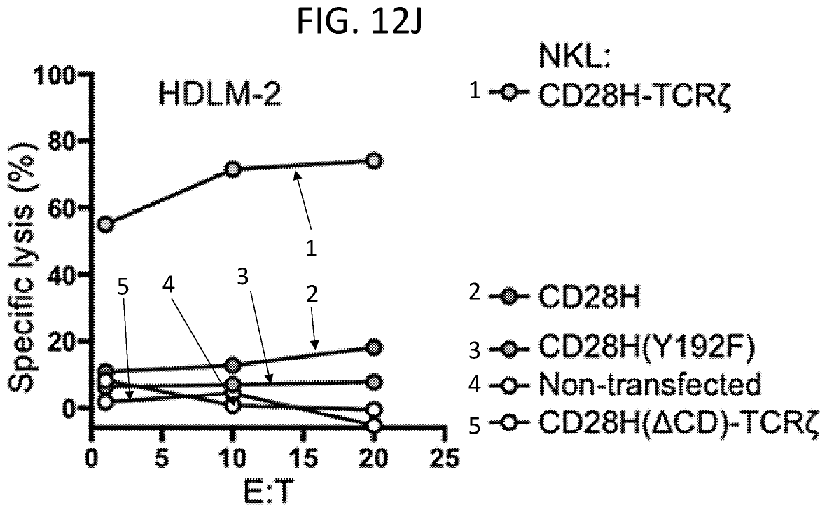

NK cells with a CD28H-CAR kill B7H7⁺ tumor cells by overriding inhibition by NKG2A: Expression of the CD28H ligand B7H7 is limited to activated myeloid cells (Zhu et al., Nat. Commun. 4; 2043, 2013; Zhao et al., PNAS 110:9879-9884, 2013) and tumor cells (Janakiram et al., Clin. Cancer Res. 21:2359-2366, 2015). We have detected marginal expression of B7H7 on monocytes and DCs activated by LPS or poly (I:C) (FIGS. 11A and 11B). The broader expression of B7H7 in tumor tissues motivated us to explore the possibility of utilizing the CD28H-B7H7 interaction to target tumors in cancer immunotherapies. We constructed two CD28H CARs by fusing full-length or cytoplasmic domain-truncated (ΔCD) CD28H with the signal transduction domain of the TCR ζ (chain (FIG. 12A and SEQ ID NOs: 1-4). Expression of the CD28H-CARs in NKL cells (FIG. 12A) did not significantly alter the cytotoxic activity toward untransfected 221 cells (FIG. 12B), and enhanced killing of 221.B7H7 cells (FIG. 12C). The nonclassical MHC-I antigen HLA-E binds inhibitory receptor NKG2A on NK cells and suppresses NK-cell activation (Long et al., Annu. Rev. Immnuol. 31:227-258, 2013). To test whether CD28H or CD28H-CARs were capable of overcoming NKG2A-mediated inhibition, 221.AEH cells, which express HLA-E at the cell surface, were transfected with B7H7 (FIG. 12D). NKL cells express NKG2A (FIG. 12E). Cytotoxicity of NKL.CD28H and NKL.CD28H-CAR cells toward 221.AEH cells was low, presumably due to inhibition by NKG2A (FIG. 12F). Remarkably, NKL.CD28H-TCRζ cells killed 221.AEH.B7H7 cells, indicating that NKG2A-mediated inhibition was overcome by the CD28H-B7H7 interaction (FIG. 12G). Expression of wild-type CD28H alone or CD28HΔCD-TCRζ chimeric receptor in NKL did not overcome NKG2A inhibition, indicating the essential costimulatory function of CD28H in the CD28H-TCRζchimera (FIG. 12G). We used the B7H7⁺ Hodgkin's lymphoma cell line HDLM-2 to further test the antitumor activity of CD28H-CARs (FIG. 12H). HDLM-2 cells, which express HLA-E (FIG. 12H), were lysed by the CD28H⁺NKG2A⁺ KHYG-1 NK cell line only after blocking the NKG2A-HLA-E interaction with a mAb to NKG2A (FIG. 12I). However, NKL cells expressing the CAR with full-length CD28H achieved efficient killing of the B7H7⁺ HLA-E⁺ HDLM-2 tumor cells (FIG. 12J), showing that CD28H-CARs have antitumor activity and therapeutic potential.

### Discussion

We showed that most NK cells in circulating blood express CD28H, a member of the CD28 receptor family that has been described in T cells. Co-engagement of CD28H with 2B4 resulted in synergistic activation of freshly isolated NK cells for degranulation, target cell lysis, and expression of cytokines and chemokines. CD28H is an addition to the family of NK cell coactivation receptors and synergizes with 2B4 and NKp46, but not with NKG2D, CD2, or DNAM-1. CD28H expression is turned off during prolonged activation with IL2. Downregulation of CD28H has also been observed in human T cells after antigenic stimulation. NK cells freshly isolated from peripheral blood, which are commonly referred to as ‘resting,’ are nevertheless fully functional even in the absence of IL2 and IL15. Co-engagement with CD28H also enhanced CD16-mediated NK-cell degranulation and cytotoxicity. Unlike coactivation receptors that require synergy, CD16 signaling in NK cells is sufficient to activate cytotoxicity. Other activation receptors, such as 2B4, CD2, NKG2D, and DNAM-1 have also been reported to enhance ADCC.

We and others have shown that B7H7 expresses on activated myeloid cells. Considering the fact that CD48 is broadly expressed on hematopoietic cells, the expression of B7H7 on activated myeloid cells might contribute to the interaction between NK cells and APCs. The involvement of other potential synergies between CD28H and other activation receptors might add another layer of complexity to the regulation of NK-APC interactions and immune homeostasis. In fact, activating receptors such as NKp30 and NKG2D have been reported as regulators of NK-DC interactions. Besides its costimulatory function, B7H7 can also have a coinhibitory effect on T cells and has been proposed as an immune checkpoint inhibitor. In the context of this Example, there was no inhibitory effect of B7H7 toward NK cell lines and primary NK cells in the functional assays performed here, including degranulation, target cell lysis, and cytokine expression. However, expression of checkpoint coinhibitory receptors, such as PD-1 and LAG-3, is usually induced or upregulated on NK cells after activation. It is possible that expression of a coinhibitory receptor for B7H7 is induced on NK cells by activation or other stimulations.

We tested the contribution of B7H7 expressed on cell lines to the activation of NK cells through CD28H. The mutant cell line 221 is sensitive to NK cell cytotoxicity due to the loss of several MHC-I ligands for NK cell inhibitory receptors. Expression of B7H7 on 221 cells rendered them even more sensitive to NK cells. This could be explained by a synergy of CD28H with 2B4, the ligand of which, CD48, is expressed on 221. However, expression of B7H7 on 221 cells that expressed also HLA-E was not sufficient to overcome inhibition by NKG2A expressed on NK cell lines, which is consistent with the dominant inhibitory function of MHC-I specific receptors on NK cells.

The signaling basis of CD28H synergy with 2B4 and NKp46 is unknown. We showed that just one of the three tyrosine residues (tyrosine 192) in the cytoplasmic tail of CD28H is required and sufficient to coactivate NK cell cytotoxicity. Substitution of the other two tyrosines with phenylalanines did not impair CD28H-mediated NK-cell activation. Tyrosine 192 and adjacent amino acids form a sequence motif (YxN) that predicts binding of SH2 domains of the adaptor Grb2 and related proteins.

We tested the possibility of using CD28H itself as a CAR by fusing it to the intracellular domain of TCRζ. Expression of the CD28H-TCRζCAR in NKL cells did not result in greater sensitivity of 221.B7H7 cells, which are already highly sensitive to lysis by NKL cells expressing CD28H. However, the CD28H-TCRζCAR in NKL cells provided complete resistance to inhibition by NKG2A, as shown with 221 cells coexpressing B7H7 and HLA-E. Resistance to inhibition was also observed with a Hodgkin's lymphoma tumor cell, HDLM-2, that naturally expresses both B7H7 and HLA-E. As expected, CD28H⁻ NKG2A⁺ NKL cells did not kill HDML-2 cells. Remarkably, expression of the CD28H-TCRζ CAR in NKL cells resulted in very efficient lysis of HDML-2 cells, whereas neither the CD28H cytoplasmic tail nor the TCRζ chain on their own could overcome inhibition. The natural transmembrane domain and cytoplasmic tail of CD28H can now be added to the list of CAR components for use in NK cells.

Solutions proposed to overcome signaling by MHC-I specific inhibitory receptors on NK cells include silencing of inhibitory receptors concomitant with CAR expression in NK cells, or combining CAR-expressing NK cells with blockade of inhibitory receptors KIR and NKG2A with antibodies, some of which are already used in the clinic. A simpler and less costly approach is to design NK-tailored CARs that overcome inhibition by KIR and NKG2A in the context of tumor cells expressing HLA-C and HLA-E. As an example, the CD28H-TCRζCAR was completely resistant to inhibition by NKG2A during contact with HLA-E⁺ tumor cells. In summary, we have shown that CD28H is an activation receptor of NK cells, and we raised the possibility of utilizing CD28H for design of NK-CARs in order to overcome signaling by inhibitory receptors and to target tumors expressing B7H7.

### Example 2

### Inhibition-Resistant Chimeric Antigen Receptor in NK Cells Delivers Strong and Persistent Activation Signals

### Materials and Methods

Cells: Isolation of human NK cells was performed as previously described. NKL cell line was obtained from M. J. Robertson (Indiana University Cancer Research Institute, Indianapolis, IN) and cultured in IMDM (Gibco) with 10% heat-inactivated fetal calf serum (FCS, Gibco). P815 cells (ATCC), 721.221 cells (referred as 221 cells), and HLA-E or HLA-C(Cw15) transfected 221 cells were cultured in RPMI-1640 (Gibco) supplemented with 10% heat-inactivated FCS.

Plasmids, lentivirus production: CAR constructs were either directly synthesized as gBlocks (IDT) or amplified by PCR from existing template, and then cloned into the EcoRI and NotI restriction sites of pCDH-EF1-T2A-Puro vector (System Biosciences). All plasmid constructions were performed using the In-Fusion HD cloning kit (Clontech). For production of lentivirus, low-passage Lenti-X 293T cells (Clontech) were transfected with PEI Max (polyethyleneimine) as described previously (Zhuang et al., Cancer Immunol. Res. 7:939-951, 2019). Culture media from Lenti-X 293T cells were collected 2 days after transfection and passed through a 0.45 μM filter. Culture media were directly used for transduction or concentrated using PEG-it (System Biosciences).

Cytotoxicity assay: Calcein-AM release assays were used to determine lysis of target cells by NK cells. Briefly, target cells at a final concentration of 10⁶/ml in complete media were incubated with 15 μM calcein-AM (Invitrogen) at 37° C. for 30 min. After 2 washes with complete media, labeled target cells were added to 96-well-plate at 10⁴/well. Effecter NK cells were added at different E:T ratio and incubated for 4 hours. Target cell lysis was determined by fluorescence of released calcein-AM in the supernatants measured by a plate reader (Enspire, Perkin Elmer, MA and SpectraMax plus, Molecular Devices, CA).

Bead stimulation: Cells were stimulated with antibody-coated goat-anti-mouse Dynabeads (Invitrogen) at the ratio of 4 beads per cell. Antibodies used for bead coating included Myc-tag antibody (9B11, mouse IgG2a, Cell Signaling), NKG2A antibody (Z199, mouse IgG2b, Beckman Coulter), control mouse IgG2b (MOPC-141, Sigma), and control mouse IgG2a (UPC-10, Sigma). 10⁷ beads were coated for 30 min at 37° C. with 2 μg total IgG at different combinations: 1 μg mIgG2a+1 μg mIgG2b, 1 μg mIgG2a+1 μg anti-NKG2A, 1 μg anti-Myc-tag+1 μg mIgG2b, or 1 μg anti-Myc-tag+1 μg anti-NKG2A. Beads were washed with 1% FCS hank's-balanced-salt-solution (HBSS, Corning) to remove excessive antibodies and resuspended in the same buffer. Pre-chilled beads and cells were mixed on ice and incubated in 37° C. water bath for 10 min or 45 min. Cells were washed and lysed before analyzing by western blot.

### Results and Discussion

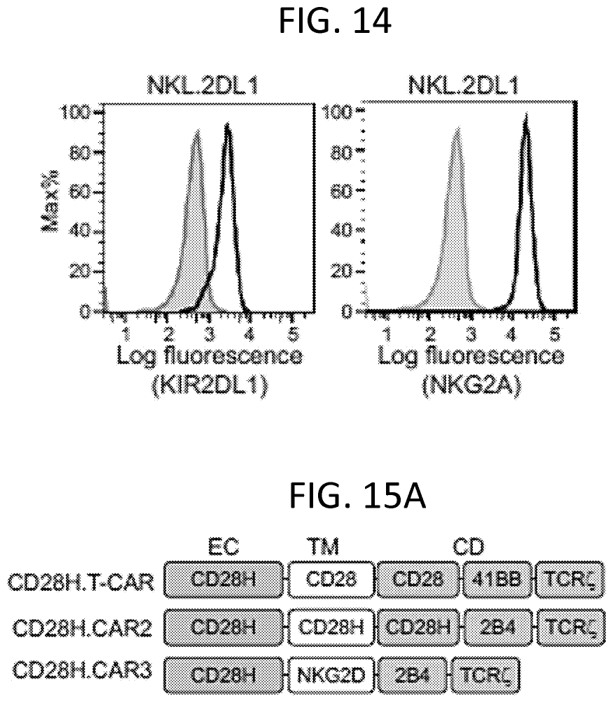

We have demonstrated that CD28H is a strong NK cell co-activation receptor, and that a chimeric receptor containing the signaling domains of both CD28H and TCRζ is resistant to NKG2A-mediated inhibition (Zhuang et al., Cancer Immunol. Res. 7:939-951, 2019). Resting human NK cells are activated by synergistic combinations of NK activation receptors (Bryceson et al., Blood 107:159-166, 2006; Long et al. Annu. Rev. Immunol. 31:227-258, 2013). As one of the combinations, synergy between CD28H and 2B4 can induce robust NK-cell activation and degranulation (FIG. 13A). We hypothesized that combining the signaling domains of CD28H and 2B4 together with TCRζ cytoplasmic domain into a CAR construct can induce potent NK cell activation, which might achieve stronger resistance to inhibitory signals. We designed CAR constructs using anti-CD19 scFv with an N-terminal Myc-tag and a CD8┘ hinge as an extracellular domain for targeting CD19⁺ cancer cells. The transmembrane domain was either from CD28 for the T-CARs, or from CD28H for the NK-tailored CARs. The intracellular domains were made of a combination of 1 to 3 signaling domains from NK receptors CD28H and 2B4, T-cell receptors CD28 and 4-1BB, and the ┐ chain of the T cell receptor (TCR-Π) (FIG. 13B). CAR constructs were transfected into NKL.2DL1 cells, which have endogenous expression of NKG2A and transgenic expression of KIR2DL1 (FIG. 14). Similar expression was achieved among different CAR constructs on NKL cells (FIG. 13C). Killing assays using the MHC-I-negative CD19⁺ 721.221 lymphoblast cell line (referred to here as 221) as target cells demonstrated that CD19.CAR2 containing the combination of the CD28H, 2B4 and CR└ signaling domains induced robust NK-cell cytotoxicity toward 221 lymphoblasts, and the signaling domains of both CD28H and 2B4 contributed to this strong activity (FIG. 13D). Compared to a third generation T-cell CAR (denoted as T-CAR), CD19.CAR2 (e.g., SEQ ID NO: 12) exhibited higher tumor-lysis efficacy toward 221 target cells (FIG. 13E).

To test resistance of the CAR construct to inhibition mediated by receptors for MHC-I, 221 cells transfected with HLA-E or HLA-C were used for cytotoxicity assays. CD19.CAR2 could overcome inhibition mediated by NKG2A or KIR2DL1 and induced lysis of MHC-I⁺ lymphoblast target cells, and both co-stimulatory domains were required for this inhibition-resistant response (FIG. 13D). By comparison to CD19.CAR2, the 3^{rd} generation CD19.T-CAR (CD28-41BB-┘) expressed in NKL.2DL1 cells was much weaker in its ability to overcome inhibition by NKG2A and KIR2DL1 (FIG. 13E). Besides the CAR constructs using CD19 scFv as the extracellular antigen-targeting domain, we also tested CARs using CD28H receptor itself as an extracellular domain for targeting B7H7⁺ tumor cells (FIGS. 15A and 15B). Killing assays were carried out using B7H7 and MHC-I (HLA-E or HLA-C) transfected 221 lymphoblasts (Zhuang et al., Cancer Immunol. Res. 7:939-951, 2019). Compared to CD28H.T-CAR, the CD28H.CAR2 (e.g., SEQ ID NO: 2) induced NK cell activation that was much more resistant to inhibition by either NKG2A or KIR2DL1 (FIGS. 15C and 15D). Moreover, CD28H.CAR2 demonstrated superior inhibition-resistant activity than another NK-tailored CAR (CAR3), composed of the transmembrane domain of NKG2D, for recruitment of the signaling molecule DAP10, fused to the cytoplasmic domains of 2B4 and TCRζ (FIGS. 15C and 15D). This NKG2D-2B4-_ transmembrane and signaling domain composition (CAR3) was previously tested as part of a mesothelin-targeting CAR in iPSC-derived NK cells (Li et al., Cell Stem Cell 23:181-192 e5, 2018).

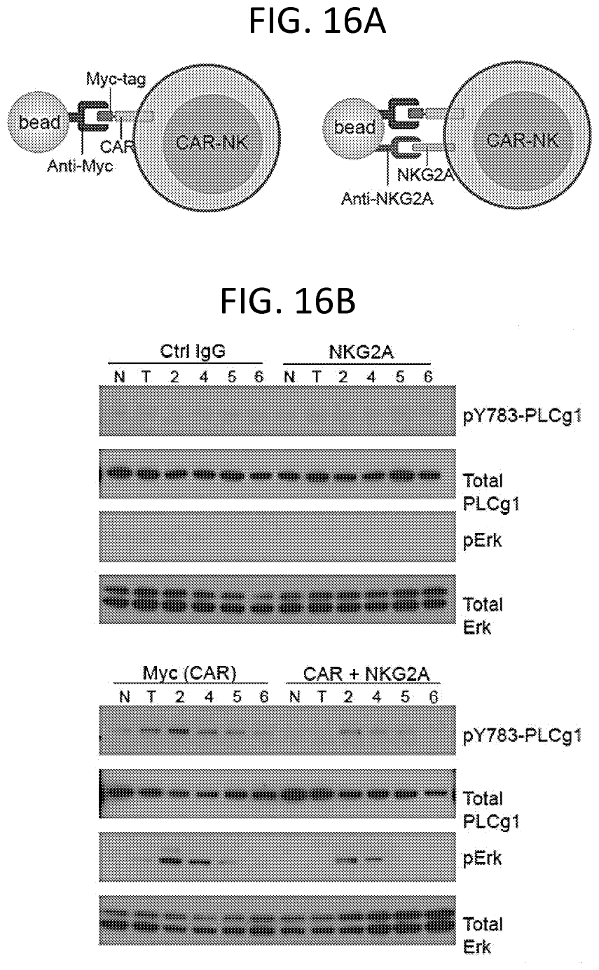

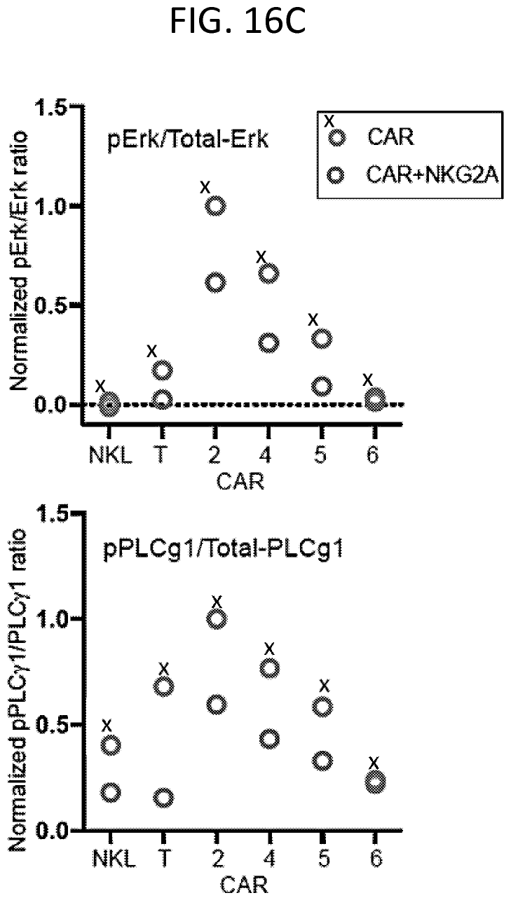

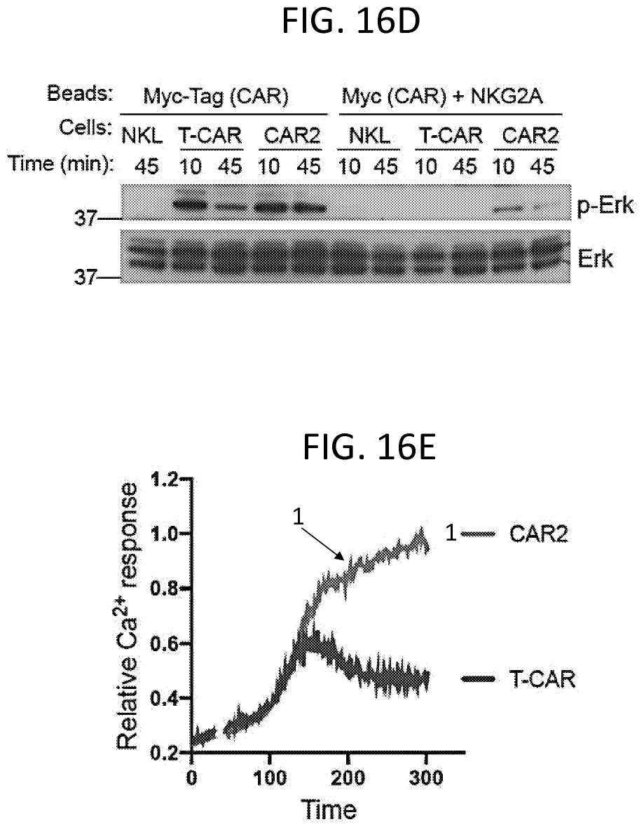

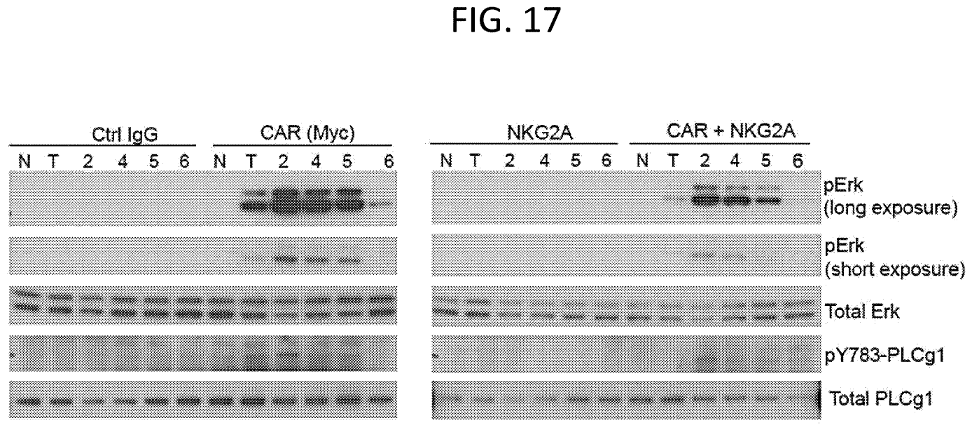

To examine activation signals transduced through CARs, NKL cells expressing CD19.CARs were stimulated by bead-coupled myc-tag antibody (Myc-beads), which induced activation signals through CARs by crosslinking N-terminal myc-tags (FIG. 16A). We evaluated phosphorylation of phospholipase Cγ1 (PLC-γ1) Tyr783 and the mitogen-associated protein kinase (MAPK/Erk) in lysates of NKL cells expressing CD19.CARs. Phosphorylation of PLC-γ1 and of ERK was detectable after stimulation through each CAR, except CAR6, which carries the cytoplasmic signaling domain of TCR└ only (FIG. 16B and FIG. 13B). Among all the tested CARs, CAR2 induced the strongest phosphorylation of both PLC-γ1 and Erk (FIGS. 16B and 16C and FIG. 15A). Stimulation with beads coupled with antibodies to either NKG2A or Myc-tag, or to both, was used to test the resistance to inhibition of different CARs (FIG. 16A). The strongest resistance to inhibition was detected after co-crosslinking NKG2A and CAR2. Substantial PLC-γ1 and Erk phosphorylation was also retained while NKG2A and CAR4 were crosslinked (FIGS. 16B and 16C). Compared to T-CAR, both CAR2 and CAR4 induced NK-cell activating signals that are more resistant to inhibition (FIGS. 16B and 16C). Moreover, sustained Erk phosphorylation was detected in CAR2-mediated NK-cell activation comparing to that of T-CAR, indicating more persistent activation signals were produced by CAR2 (FIG. 16D). This conclusion was further supported by calcium mobilization assays, which showed more sustained calcium mobilization after crosslinking of CAR2 than T-CAR (FIG. 16E).

Here we demonstrated that the NK-tailored CAR2 containing a fused signaling domain of CD28H-2B4-TCRζ was a more effective CAR construct for expression in NK cells than the third-generation T-CAR. CAR2 induced stronger cytotoxicity and activation signals than T-CAR. Importantly, CAR-2 triggered inhibition-resistant activation signals, which facilitated NK cells to overcome inhibition mediated by receptors for MHC class I and kill MHC class I⁺ cancer cells. Although some transformed cancer cells down regulate MHC class I to escape T-cell-mediated surveillance, many tumor cells still retain substantial expression of MHC class I, which could engage NKG2A or inhibitory KIRs and limit anti-cancer responses of NK cells. These results support that, unlike cellular therapy with T cells, evaluation of inhibition-resistance property of CAR constructs for expression in NK cells is important. Design of CAR2 signaling domain was based on the strong synergy between activating receptors 2B4 and CD28H.

### Example 3

### In Vivo Evaluation of CAR Efficacy

This example describes methods that can be used to evaluate efficacy of one or more of the disclosed CARs in vivo in a mouse xenograft model. However, one skilled in the art will appreciate that methods that deviate from these specific methods can also be used to test efficacy of CARs in vivo.

NOD-scid IL2R [symbol] ^{null} mice (also known as NSG mice) are injected with 10⁵ to 10⁶ luciferase-expressing NALM-6 B-ALL cells. NK cells are isolated from the PBMCs of healthy donors by positive and/or negative selection using immune-magnetic methods. Expanded NK cells are transduced with a retroviral vector including a CD19-CAR, such as those described herein (e.g., SEQ ID NOs: 1, 5, 11, and 15). A composition comprising 10⁶ to 10⁷ of the transduced and expanded NK cells is administered to tumor-bearing mice intravenously. In vivo proliferation and persistence of CAR-expressing NK cells is supported by administration of human IL-2 or IL-15. Tumor burden is tracked by bioluminescent imaging (BLI). In vivo efficacy of CARs is evaluated by both tumor burden and overall survival (OS) of tumor-bearing NSG mice.

### Example 4

Treating B-Cell Lymphoma with CD19-CAR Expressing NK Cells

This example describes methods that can be used to treat a subject with a B-cell lymphoma with CD19-CAR transduced NK cells. However, one skilled in the art will appreciate that methods that deviate from these specific methods can also be used to successfully treat a subject with B-cell lymphoma. In addition, similar methods can be used to treat a subject with other CAR-expressing NK cells and other cancers.

A subject with a B-cell lymphoma undergoes apheresis to collect peripheral blood mononuclear cells. NK cells (e.g., CD56-positive/CD3-negative cells) are isolated from the PBMCs by positive and/or negative selection using immune-magnetic methods, transduced with a retroviral vector including a CD19-CAR, such as those described herein (e.g., SEQ ID NOs: 1, 5, 11, 15, and 19). The transduced NK cells can be cryopreserved for later use or can be formulated for administration to the subject (for example, in a pharmaceutically acceptable carrier). A composition comprising 10⁶ to 10¹² of the expanded NK cells is administered to the subject intravenously.

In view of the many possible embodiments to which the principles of the disclosure may be applied, it should be recognized that the illustrated embodiments are only examples and should not be taken as limiting the scope of the invention. Rather, the scope of the invention is defined by the following claims. We therefore claim as our invention all that comes within the scope and spirit of these claims.

We claim:

1. A nucleic acid molecule encoding a chimeric antigen receptor comprising:
(i) (a) a target binding domain;
(b) a transmembrane domain; and
(c) an intracellular domain comprising a first intracellular signaling domain from CD28 homolog (CD28H) and a second intracellular signaling domain, wherein the first and second intracellular signaling domains can be in either order; or
(ii) (a) a target binding domain;
(b) a transmembrane domain; and
(c) an intracellular domain comprising a first intracellular signaling domain from CD28 homolog (CD28H), a second intracellular signaling domain from 2B4, and a third intracellular signaling domain, wherein the first, second, and third intracellular signaling domains can be in any order.

2. The nucleic acid molecule of claim 1, wherein the nucleic acid encodes a chimeric antigen receptor wherein the CD28H intracellular signaling domain comprises an amino acid sequence with at least 90% identity to the amino acids 172-282 of SEQ ID NO: 2 or comprises the amino acid sequence of amino acids 172-282 of SEQ ID NO: 2.

3. The nucleic acid molecule of claim 1(i), wherein the nucleic acid encodes a second intracellular signaling domain from 2B4, TCRζ, FcεR1γ, or DAP12.

4. The nucleic acid molecule of claim 3, wherein the nucleic acid encodes:
(a) a TCRζ intracellular signaling domain comprising an amino acid sequence with at least 90% identity to the amino acids 283-395 of SEQ ID NO: 2 or comprising the amino acid sequence of amino acids 283-395 of SEQ ID NO: 2; or
(b) a 2B4 intracellular signaling domain comprising an amino acid sequence with at least 90% identity to the amino acids 172-291 of SEQ ID NO: 10 or comprising the amino acid sequence of amino acids 172-291 of SEQ ID NO: 10.

5. The nucleic acid molecule of claim 1, wherein the nucleic acid encodes an intracellular domain further comprising an intracellular region between the transmembrane domain and the first intracellular signaling domain.

6. The nucleic acid molecule of claim 1, wherein the nucleic acid encodes:
(a) a transmembrane domain and intracellular region from CD16, NKp46, NKp30, NKp44, or KIR2DS4;
(b) a CD19 scFv or a CD28H extracellular domain; and/or
(c) a CD28H transmembrane domain.

7. The nucleic acid molecule of claim 1(ii), wherein nucleic acid encodes a third intracellular signaling domain from TCRζ, FcεR1γ, or DAP12.

8. The nucleic acid molecule of claim 1, wherein the nucleic acid further encodes:
(a) a hinge domain, wherein the hinge domain is C-terminal to the target binding domain and N-terminal to the transmembrane domain; and/or
(b) an amino-terminal signal sequence.

9. The nucleic acid molecule of claim 1, wherein the nucleic acid encodes an amino acid sequence with at least 90% sequence identity to the amino acid sequence of any one of SEQ ID NOs: 2, 6, 12, 16, and 20 or an amino acid sequence comprising the amino acid sequence of any one of SEQ ID NOs: 2, 6, 12, 16, and 20.

10. The nucleic acid molecule of claim 1, comprising a nucleic acid sequence with at least 90% sequence identity to the nucleic acid sequence of any one of SEQ ID NOs: 1, 5, 11, 15, and 19 comprising the nucleic acid sequence of any one of SEQ ID NOs: 1, 5, 11, 15, and 19.

11. A vector comprising the nucleic acid molecule of claim 1.

12. The vector of claim 11, wherein the vector is a viral vector.

13. The vector of claim 12, wherein the viral vector is a lentiviral vector or a retrovirus vector.

14. A cell comprising the nucleic acid molecule of claim 1 or a vector comprising the nucleic acid molecule.

15. The cell of claim 14 wherein the cell is a natural killer cell or a T cell.

16. A composition comprising the nucleic acid molecule of claim 1 or a cell comprising the nucleic acid molecule, and a pharmaceutically acceptable carrier.

17. A method of treating a subject with cancer, comprising administering to the subject an effective amount of the cell of claim 14.

18. The method of claim 17, wherein the cell comprises natural killer cells or T cells.

19. The method of claim 18, wherein the natural killer cells or T cells are autologous to the subject being treated.

20. The method of claim 17, wherein the cancer is a hematological malignancy or a solid tumor.


SEQUENCE LISTING (25 sequences)

SEQ ID NO: 1 - DNA, 1185 bp, synthetic construct
  misc_feature 1..1185: note = CD28H(FL)-TCRzeta(CD)
  source 1..1185: mol_type = other DNA
atggggtccccgggcatggtgctgggcctcctggtgcagatctgggccctgcaagaagcctcaagcctgagcgtgcagcaggggcccaacttgctgcaggtgaggcagggcagtcaggcgaccctggtctgccaggtggaccaggccacagcctgggaacggctccgtgttaagtggacaaaggatggggccatcctgtgtcaaccgtacatcaccaacggcagcctcagcctgggggtctgcgggccccagggacggctctcctggcaggcacccagccatctcaccctgcagctggaccctgtgagcctcaaccacagcggggcgtacgtgtgctgggcggccgtagagattcctgagttggaggaggctgagggcaacataacaaggctctttgtggacccagatgaccccacacagaacagaaaccggatcgcaagcttcccaggattcctcttcgtgctgctgggggtgggaagcatgggtgtggctgcgatcgtgtggggtgcctggttctggggccgccgcagctgccagcaaagggactcaggtaacagcccaggaaatgcattctacagcaacgtcctataccggccccggggggccccaaagaagagtgaggactgctctggagaggggaaggaccagaggggccagagcatttattcaacctccttcccgcaaccggccccccgccagccgcacctggcgtcaagaccctgccccagcccgagaccctgccccagccccaggcccggccaccccgtctctatggtcagggtctctcctagaccaagccccacccagcagccgaggccaaaagggttccccaaagtgggagaggagagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 2 - protein, 395 aa, synthetic construct
  REGION 1..395: note = CD28H(FL)-TCRzeta(CD)
MGSPGMVLGLLVQIWALQEASSLSVQQGPNLLQVRQGSQATLVCQVDQATAWERLRVKWTKDGAILCQPYITNGSLSLGVCGPQGRLSWQAPSHLTLQLDPVSLNHSGAYVCWAAVEIPELEEAEGNITRLFVDPDDPTQNRNRIASFPGFLFVLLGVGSMGVAAIVWGAWFWGRRSCQQRDSGNSPGNAFYSNVLYRPRGAPKKSEDCSGEGKDQRGQSIYSTSFPQPAPRQPHLASRPCPSPRPCPSPRPGHPVSMVRVSPRPSPTQQPRPKGFPKVGEERVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 3 - DNA, 852 bp, synthetic construct
  misc_feature 1..852: note = CD28H(EC-TM)-TCRzeta(CD)
  source 1..852: mol_type = other DNA
atggggtccccgggcatggtgctgggcctcctggtgcagatctgggccctgcaagaagcctcaagcctgagcgtgcagcaggggcccaacttgctgcaggtgaggcagggcagtcaggcgaccctggtctgccaggtggaccaggccacagcctgggaacggctccgtgttaagtggacaaaggatggggccatcctgtgtcaaccgtacatcaccaacggcagcctcagcctgggggtctgcgggccccagggacggctctcctggcaggcacccagccatctcaccctgcagctggaccctgtgagcctcaaccacagcggggcgtacgtgtgctgggcggccgtagagattcctgagttggaggaggctgagggcaacataacaaggctctttgtggacccagatgaccccacacagaacagaaaccggatcgcaagcttcccaggattcctcttcgtgctgctgggggtgggaagcatgggtgtggctgcgatcgtgtggggtgcctggagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 4 - protein, 284 aa, synthetic construct
  REGION 1..284: note = CD28H(EC-TM)-TCRzeta(CD)
MGSPGMVLGLLVQIWALQEASSLSVQQGPNLLQVRQGSQATLVCQVDQATAWERLRVKWTKDGAILCQPYITNGSLSLGVCGPQGRLSWQAPSHLTLQLDPVSLNHSGAYVCWAAVEIPELEEAEGNITRLFVDPDDPTQNRNRIASFPGFLFVLLGVGSMGVAAIVWGAWRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 5 - DNA, 1545 bp, synthetic construct
  misc_feature 1..1545: note = CD28H.CAR2
  source 1..1545: mol_type = other DNA
atggggtccccgggcatggtgctgggcctcctggtgcagatctgggccctgcaagaagcctcaagcctgagcgtgcagcaggggcccaacttgctgcaggtgaggcagggcagtcaggcgaccctggtctgccaggtggaccaggccacagcctgggaacggctccgtgttaagtggacaaaggatggggccatcctgtgtcaaccgtacatcaccaacggcagcctcagcctgggggtctgcgggccccagggacggctctcctggcaggcacccagccatctcaccctgcagctggaccctgtgagcctcaaccacagcggggcgtacgtgtgctgggcggccgtagagattcctgagttggaggaggctgagggcaacataacaaggctctttgtggacccagatgaccccacacagaacagaaaccggatcgcaagcttcccaggattcctcttcgtgctgctgggggtgggaagcatgggtgtggctgcgatcgtgtggggtgcctggttctggggccgccgcagctgccagcaaagggactcaggtaacagcccaggaaatgcattctacagcaacgtcctataccggccccggggggccccaaagaagagtgaggactgctctggagaggggaaggaccagaggggccagagcatttattcaaccagtttccctcaaccggctccaagacaaccacatctcgccagtcggccttgtccgtcccctagaccctgccccagtcccaggcccggccaccccgtctctatggtcagggtctctcctagaccaagccccacccagcagccgaggccaaaagggttccccaaagtgggagaggagtggaggagaaagaggaaggagaagcagtcagagaccagtcccaaggaatttttgacaatttacgaagatgtcaaggatctgaaaaccaggagaaatcacgagcaggagcagacttttcctggaggggggagcaccatctactctatgatccagtcccagtcttctgctcccacgtcacaagaacctgcatatacattatattcattaattcagccttccaggaagtctggatccaggaagaggaaccacagcccttccttcaatagcactatctatgaagtgattggaaagagtcaacctaaagcccagaaccctgctcgattgagccgcaaagagctggagaactttgatgtttattccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 6 - protein, 515 aa, synthetic construct
  REGION 1..515: note = CD28H.CAR2
MGSPGMVLGLLVQIWALQEASSLSVQQGPNLLQVRQGSQATLVCQVDQATAWERLRVKWTKDGAILCQPYITNGSLSLGVCGPQGRLSWQAPSHLTLQLDPVSLNHSGAYVCWAAVEIPELEEAEGNITRLFVDPDDPTQNRNRIASFPGFLFVLLGVGSMGVAAIVWGAWFWGRRSCQQRDSGNSPGNAFYSNVLYRPRGAPKKSEDCSGEGKDQRGQSIYSTSFPQPAPRQPHLASRPCPSPRPCPSPRPGHPVSMVRVSPRPSPTQQPRPKGFPKVGEEWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 7 - DNA, 1119 bp, synthetic construct
  misc_feature 1..1119: note = CD28H.T-CAR
  source 1..1119: mol_type = other DNA
atggggtccccgggcatggtgctgggcctcctggtgcagatctgggccctgcaagaagcctcaagcctgagcgtgcagcaggggcccaacttgctgcaggtgaggcagggcagtcaggcgaccctggtctgccaggtggaccaggccacagcctgggaacggctccgtgttaagtggacaaaggatggggccatcctgtgtcaaccgtacatcaccaacggcagcctcagcctgggggtctgcgggccccagggacggctctcctggcaggcacccagccatctcaccctgcagctggaccctgtgagcctcaaccacagcggggcgtacgtgtgctgggcggccgtagagattcctgagttggaggaggctgagggcaacataacaaggctctttgtggacccagatgaccccacacagaacagaaaccggatcgcaagcttcccaggattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 8 - protein, 373 aa, synthetic construct
  REGION 1..373: note = CD28H.T-CAR
MGSPGMVLGLLVQIWALQEASSLSVQQGPNLLQVRQGSQATLVCQVDQATAWERLRVKWTKDGAILCQPYITNGSLSLGVCGPQGRLSWQAPSHLTLQLDPVSLNHSGAYVCWAAVEIPELEEAEGNITRLFVDPDDPTQNRNRIASFPGFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 9 - DNA, 1212 bp, synthetic construct
  misc_feature 1..1212: note = CD28H.CAR3
  source 1..1212: mol_type = other DNA
atggggtccccgggcatggtgctgggcctcctggtgcagatctgggccctgcaagaagcctcaagcctgagcgtgcagcaggggcccaacttgctgcaggtgaggcagggcagtcaggcgaccctggtctgccaggtggaccaggccacagcctgggaacggctccgtgttaagtggacaaaggatggggccatcctgtgtcaaccgtacatcaccaacggcagcctcagcctgggggtctgcgggccccagggacggctctcctggcaggcacccagccatctcaccctgcagctggaccctgtgagcctcaaccacagcggggcgtacgtgtgctgggcggccgtagagattcctgagttggaggaggctgagggcaacataacaaggctctttgtggacccagatgaccccacacagaacagaaaccggatcgcaagcttcccaggaccattttttttctgctgcttcatcgctgtagccatgggaatccgtttcattattatggtaacatggaggagaaagaggaaggagaagcagtcagagaccagtcccaaggaatttttgacaatttacgaagatgtcaaggatctgaaaaccaggagaaatcacgagcaggagcagacttttcctggaggggggagcaccatctactctatgatccagtcccagtcttctgctcccacgtcacaagaacctgcatatacattatattcattaattcagccttccaggaagtctggatccaggaagaggaaccacagcccttccttcaatagcactatctatgaagtgattggaaagagtcaacctaaagcccagaaccctgctcgattgagccgcaaagagctggagaactttgatgtttattccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 10 - protein, 404 aa, synthetic construct
  REGION 1..404: note = CD28H.CAR3
MGSPGMVLGLLVQIWALQEASSLSVQQGPNLLQVRQGSQATLVCQVDQATAWERLRVKWTKDGAILCQPYITNGSLSLGVCGPQGRLSWQAPSHLTLQLDPVSLNHSGAYVCWAAVEIPELEEAEGNITRLFVDPDDPTQNRNRIASFPGPFFFCCFIAVAMGIRFIIMVTWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 11 - DNA, 2049 bp, synthetic construct
  misc_feature 1..2049: note = CD19.CAR2
  source 1..2049: mol_type = other DNA
atggcgctccctgtcaccgcactgcttcttccgctggcactgctgctgcacgctgcacggcctgagcaaaaacttatctctgaagaggacctcgatatacagatgacgcagacaacgtcaagtctttccgccagcttgggagaccgagtgactatatcttgtagagcaagccaggatatttctaagtatcttaactggtaccaacaaaagcccgatggaacggttaagctgcttatataccataccagtagactccactccggcgtaccatcacggttttctggcagtggctccgggaccgactattctttgacgatctctaatctcgaacaagaggatattgcaacatacttttgtcagcaaggcaataccttgccatatacgtttgggggcgggacaaaacttgagataaccggcggcggtggttcaggcggtggcggttccggtggtgggggatcagaggttaagcttcaggaatccggaccaggtttggttgcccccagccaatctctcagcgttacatgcacggtttcaggcgtcagtctccccgattacggtgtaagttggattcggcaacctccgcgaaagggtctggaatggctgggggttatttgggggagtgagacaacttattacaactctgcacttaagagtcggcttaccatcatcaaggataattcaaaatcacaagtattcctgaagatgaactcattgcaaacagatgatacagctatatactattgtgccaagcattactattatggtggttcttatgcaatggattactgggggcaaggcacgtcagtgacagtgagttcaaccaccacccctgcaccaagacctccaactcctgccccaactattgcaagtcagccactttctttgcgaccagaggcgtgccggccagcggcggggggcgcagtgcacacgagggggctggacttcgcctgtgatttcctcttcgtgctgctgggggtgggaagcatgggtgtggctgcgatcgtgtggggtgcctggttctggggccgccgcagctgccagcaaagggactcaggtaacagcccaggaaatgcattctacagcaacgtcctataccggccccggggggccccaaagaagagtgaggactgctctggagaggggaaggaccagaggggccagagcatttattcaaccagtttccctcaaccggctccaagacaaccacatctcgccagtcggccttgtccgtcccctagaccctgccccagtcccaggcccggccaccccgtctctatggtcagggtctctcctagaccaagccccacccagcagccgaggccaaaagggttccccaaagtgggagaggagtggaggagaaagaggaaggagaagcagtcagagaccagtcccaaggaatttttgacaatttacgaagatgtcaaggatctgaaaaccaggagaaatcacgagcaggagcagacttttcctggaggggggagcaccatctactctatgatccagtcccagtcttctgctcccacgtcacaagaacctgcatatacattatattcattaattcagccttccaggaagtctggatccaggaagaggaaccacagcccttccttcaatagcactatctatgaagtgattggaaagagtcaacctaaagcccagaaccctgctcgattgagccgcaaagagctggagaactttgatgtttattccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 12 - protein, 683 aa, synthetic construct
  REGION 1..683: note = CD19.CAR2
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDFLFVLLGVGSMGVAAIVWGAWFWGRRSCQQRDSGNSPGNAFYSNVLYRPRGAPKKSEDCSGEGKDQRGQSIYSTSFPQPAPRQPHLASRPCPSPRPCPSPRPGHPVSMVRVSPRPSPTQQPRPKGFPKVGEEWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 13 - DNA, 1623 bp, synthetic construct
  misc_feature 1..1623: note = CD19-T-CAR
  source 1..1623: mol_type = other DNA
atggcgctccctgtcaccgcactgcttcttccgctggcactgctgctgcacgctgcacggcctgagcaaaaacttatctctgaagaggacctcgatatacagatgacgcagacaacgtcaagtctttccgccagcttgggagaccgagtgactatatcttgtagagcaagccaggatatttctaagtatcttaactggtaccaacaaaagcccgatggaacggttaagctgcttatataccataccagtagactccactccggcgtaccatcacggttttctggcagtggctccgggaccgactattctttgacgatctctaatctcgaacaagaggatattgcaacatacttttgtcagcaaggcaataccttgccatatacgtttgggggcgggacaaaacttgagataaccggcggcggtggttcaggcggtggcggttccggtggtgggggatcagaggttaagcttcaggaatccggaccaggtttggttgcccccagccaatctctcagcgttacatgcacggtttcaggcgtcagtctccccgattacggtgtaagttggattcggcaacctccgcgaaagggtctggaatggctgggggttatttgggggagtgagacaacttattacaactctgcacttaagagtcggcttaccatcatcaaggataattcaaaatcacaagtattcctgaagatgaactcattgcaaacagatgatacagctatatactattgtgccaagcattactattatggtggttcttatgcaatggattactgggggcaaggcacgtcagtgacagtgagttcaaccaccacccctgcaccaagacctccaactcctgccccaactattgcaagtcagccactttctttgcgaccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 14 - protein, 541 aa, synthetic construct
  REGION 1..541: note = CD19.T-CAR
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 15 - DNA, 1689 bp, synthetic construct
  misc_feature 1..1689: note = CD19.CAR4
  source 1..1689: mol_type = other DNA
atggcgctccctgtcaccgcactgcttcttccgctggcactgctgctgcacgctgcacggcctgagcaaaaacttatctctgaagaggacctcgatatacagatgacgcagacaacgtcaagtctttccgccagcttgggagaccgagtgactatatcttgtagagcaagccaggatatttctaagtatcttaactggtaccaacaaaagcccgatggaacggttaagctgcttatataccataccagtagactccactccggcgtaccatcacggttttctggcagtggctccgggaccgactattctttgacgatctctaatctcgaacaagaggatattgcaacatacttttgtcagcaaggcaataccttgccatatacgtttgggggcgggacaaaacttgagataaccggcggcggtggttcaggcggtggcggttccggtggtgggggatcagaggttaagcttcaggaatccggaccaggtttggttgcccccagccaatctctcagcgttacatgcacggtttcaggcgtcagtctccccgattacggtgtaagttggattcggcaacctccgcgaaagggtctggaatggctgggggttatttgggggagtgagacaacttattacaactctgcacttaagagtcggcttaccatcatcaaggataattcaaaatcacaagtattcctgaagatgaactcattgcaaacagatgatacagctatatactattgtgccaagcattactattatggtggttcttatgcaatggattactgggggcaaggcacgtcagtgacagtgagttcaaccaccacccctgcaccaagacctccaactcctgccccaactattgcaagtcagccactttctttgcgaccagaggcgtgccggccagcggcggggggcgcagtgcacacgagggggctggacttcgcctgtgatttcctcttcgtgctgctgggggtgggaagcatgggtgtggctgcgatcgtgtggggtgcctggttctggggccgccgcagctgccagcaaagggactcaggtaacagcccaggaaatgcattctacagcaacgtcctataccggccccggggggccccaaagaagagtgaggactgctctggagaggggaaggaccagaggggccagagcatttattcaaccagtttccctcaaccggctccaagacaaccacatctcgccagtcggccttgtccgtcccctagaccctgccccagtcccaggcccggccaccccgtctctatggtcagggtctctcctagaccaagccccacccagcagccgaggccaaaagggttccccaaagtgggagaggagagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 16 - protein, 563 aa, synthetic construct
  REGION 1..563: note = CD19.CAR4
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDFLFVLLGVGSMGVAAIVWGAWFWGRRSCQQRDSGNSPGNAFYSNVLYRPRGAPKKSEDCSGEGKDQRGQSIYSTSFPQPAPRQPHLASRPCPSPRPCPSPRPGHPVSMVRVSPRPSPTQQPRPKGFPKVGEERVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 17 - DNA, 1497 bp, synthetic construct
  misc_feature 1..1497: note = CD19.CAR5
  source 1..1497: mol_type = other DNA
atggcgctccctgtcaccgcactgcttcttccgctggcactgctgctgcacgctgcacggcctgagcaaaaacttatctctgaagaggacctcgatatacagatgacgcagacaacgtcaagtctttccgccagcttgggagaccgagtgactatatcttgtagagcaagccaggatatttctaagtatcttaactggtaccaacaaaagcccgatggaacggttaagctgcttatataccataccagtagactccactccggcgtaccatcacggttttctggcagtggctccgggaccgactattctttgacgatctctaatctcgaacaagaggatattgcaacatacttttgtcagcaaggcaataccttgccatatacgtttgggggcgggacaaaacttgagataaccggcggcggtggttcaggcggtggcggttccggtggtgggggatcagaggttaagcttcaggaatccggaccaggtttggttgcccccagccaatctctcagcgttacatgcacggtttcaggcgtcagtctccccgattacggtgtaagttggattcggcaacctccgcgaaagggtctggaatggctgggggttatttgggggagtgagacaacttattacaactctgcacttaagagtcggcttaccatcatcaaggataattcaaaatcacaagtattcctgaagatgaactcattgcaaacagatgatacagctatatactattgtgccaagcattactattatggtggttcttatgcaatggattactgggggcaaggcacgtcagtgacagtgagttcaaccaccacccctgcaccaagacctccaactcctgccccaactattgcaagtcagccactttctttgcgaccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 18 - protein, 499 aa, synthetic construct
  REGION 1..499: note = CD19.CAR5
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 19 - DNA, 1356 bp, synthetic construct
  misc_feature 1..1356: note = CD19.CAR6
  source 1..1356: mol_type = other DNA
atggcgctccctgtcaccgcactgcttcttccgctggcactgctgctgcacgctgcacggcctgagcaaaaacttatctctgaagaggacctcgatatacagatgacgcagacaacgtcaagtctttccgccagcttgggagaccgagtgactatatcttgtagagcaagccaggatatttctaagtatcttaactggtaccaacaaaagcccgatggaacggttaagctgcttatataccataccagtagactccactccggcgtaccatcacggttttctggcagtggctccgggaccgactattctttgacgatctctaatctcgaacaagaggatattgcaacatacttttgtcagcaaggcaataccttgccatatacgtttgggggcgggacaaaacttgagataaccggcggcggtggttcaggcggtggcggttccggtggtgggggatcagaggttaagcttcaggaatccggaccaggtttggttgcccccagccaatctctcagcgttacatgcacggtttcaggcgtcagtctccccgattacggtgtaagttggattcggcaacctccgcgaaagggtctggaatggctgggggttatttgggggagtgagacaacttattacaactctgcacttaagagtcggcttaccatcatcaaggataattcaaaatcacaagtattcctgaagatgaactcattgcaaacagatgatacagctatatactattgtgccaagcattactattatggtggttcttatgcaatggattactgggggcaaggcacgtcagtgacagtgagttcaaccaccacccctgcaccaagacctccaactcctgccccaactattgcaagtcagccactttctttgcgaccagaggcgtgccggccagcggcggggggcgcagtgcacacgagggggctggacttcgcctgtgatttcctcttcgtgctgctgggggtgggaagcatgggtgtggctgcgatcgtgtggggtgcctggagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 20 - protein, 452 aa, synthetic construct
  REGION 1..452: note = CD19.CAR6
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDFLFVLLGVGSMGVAAIVWGAWRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 21 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 22 - protein, 304 aa, Homo sapiens
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLLRMGLAFLVLVALVWFLVEDWLSRKRTRERASRASTWEGRRRLNTQTL

SEQ ID NO: 23 - protein, 201 aa, Homo sapiens
MAWMLLLILIMVHPGSCALWVSQPPEIRTLEGSSAFLPCSFNASQGRLAIGSVTWFRDEVVPGKEVRNGTPEFRGRLAPLASSRFLHDHQAELHIRDVRGHDASIYVCRVEVLGLGVGTGNGTRLVVEKEHPQLGAGTVLLLRAGFYAVSFLSVAVGSTVYYQGKCLTWKGPRRQLPAVVPAPLPPPCGSSAHLLPPVPGG

SEQ ID NO: 24 - protein, 276 aa, Homo sapiens
MAWRALHPLLLLLLLFPGSQAQSKAQVLQSVAGQTLTVRCQYPPTGSLYEKKGWCKEASALVCIRLVTSSKPRTMAWTSRFTIWDDPDAGFFTVTMTDLREEDSGHYWCRIYRPSDNSVSKSVRFYLVVSPASASTQTSWTPRDLVSSQTQTQSCVPPTAGARQAPESPSTIPVPSQPQNSTLRPGPAAPIALVPVFCGLLVAKSLVLSALLVWWGDIWWKTMMELRSLDTQKATCHLQQVTDLPWTSVSSPVEREILYHTVARTKISDDDDEHTL

SEQ ID NO: 25 - protein, 304 aa, Homo sapiens
MSLMVIIMACVGFFLLQGAWPQEGVHRKPSFLALPGHLVKSEETVILQCWSDVMFEHFLLHREGKFNNTLHLIGEHHDGVSKANFSIGPMMPVLAGTYRCYGSVPHSPYQLSAPSDPLDMVIIGLYEKPSLSAQPGPTVQAGENVTLSCSSRSSYDMYHLSREGEAHERRLPAVRSINGTFQADFPLGPATHGGTYRCFGSFRDAPYEWSNSSDPLLVSVTGNPSNSWPSPTEPSSKTGNPRHLHVLIGTSVVKIPFTILLFFLLHRWCSDKKNAAVMDQEPAGNRTVNSEDSDEQDHQEVSYA